(19) United States
(12) Patent Application Publication
(10) Pub. No.: US 2024/0150825 A1
(43) Pub. Date: May 9, 2024

(54) METHODS AND COMPOSITIONS FOR ANALYZING NUCLEIC ACID

(71) Applicant: Claret Bioscience, LLC, Santa Cruz, CA (US)

(72) Inventor: Charles VASKE, Santa Cruz, CA (US)

(21) Appl. No.: 18/280,588

(22) PCT Filed: Mar. 8, 2022
(86) PCT No.: PCT/US2022/019255

§ 371 Date: Sep. 6, 2023

Related U.S. Application Data
(60) Provisional application No. 63/307,511, filed on Feb. 7, 2022.
(60) Provisional application No. 63/277,906, filed on Nov. 10, 2021.
(60) Provisional application No. 63/161,549, filed on Mar. 16, 2021.
(60) Provisional application No. 63/158,574, filed on Mar. 9, 2021.

(51) Int. Cl. C12Q 1/6869; G16B 30/00

(52) U.S. Cl. CPC C12Q 1/6869; G16B 30/00

(57) ABSTRACT

The technology relates in part to methods and compositions for analyzing nucleic acid. In some aspects, the technology relates to methods and compositions for preparing a nucleic acid library from single-stranded nucleic acid fragments. In some aspects, the technology relates to methods and compositions for analyzing a nucleic acid fragment length profile in a sample.

### RELATED PATENT APPLICATION(S)

This patent application claims the benefit of U.S. provisional patent application No. 63/158,574 filed on Mar. 9, 2021, entitled METHODS AND COMPOSITIONS FOR ANALYZING NUCLEIC ACID, naming Charles VASKE as inventor, and designated by attorney docket no. CBS-2007-PV. This patent application also claims the benefit of U.S. provisional patent application No. 63/161,549 filed on Mar. 16, 2021, entitled METHODS AND COMPOSITIONS FOR ANALYZING NUCLEIC ACID, naming Charles VASKE as inventor, and designated by attorney docket no. CBS-2007-PV2. This patent application also claims the benefit of U.S. provisional patent application No. 63/277,906 filed on Nov. 10, 2021, entitled METHODS AND COMPOSITIONS FOR ANALYZING NUCLEIC ACID, naming Charles VASKE as inventor, and designated by attorney docket no. CBS-2007-PV3. This patent application also claims the benefit of U.S. provisional patent application No. 63/307,511 filed on Feb. 7, 2022, entitled METHODS AND COMPOSITIONS FOR ANALYZING NUCLEIC ACID, naming Charles VASKE as inventor, and designated by attorney docket no. CBS-2007-PV4. The entire content of the foregoing patent applications is incorporated herein by reference for all purposes, including all text, tables and drawings.

### FIELD

The technology relates in part to methods and compositions for analyzing nucleic acid. In some aspects, the technology relates to methods and compositions for preparing a nucleic acid library from single-stranded nucleic acid fragments. In some aspects, the technology relates to methods and compositions for analyzing a nucleic acid fragment length profile in a sample. In some aspects, the technology relates to methods and compositions for detecting a population of cells in a subject according to nucleic acid fragment lengths.

### BACKGROUND

Genetic information of living organisms (e.g., animals, plants and microorganisms) and other forms of replicating genetic information (e.g., viruses) is encoded in nucleic acid (i.e., deoxyribonucleic acid (DNA) or ribonucleic acid (RNA)). Genetic information is a succession of nucleotides or modified nucleotides representing the primary structure of chemical or hypothetical nucleic acids.

A variety of high-throughput sequencing platforms are used for analyzing nucleic acid. The ILLUMINA platform, for example, involves clonal amplification of adaptor-ligated DNA fragments. Another platform is nanopore-based sequencing, which relies on the transition of nucleic acid molecules or individual nucleotides through a small channel. Library preparation for certain sequencing platforms often includes fragmentation of DNA, modification of fragment ends, and ligation of adapters, and may include amplification of nucleic acid fragments (e.g., PCR amplification).

The selection of an appropriate sequencing platform for particular types of nucleic acid analysis requires a detailed understanding of the technologies available, including sources of error, error rate, as well as the speed and cost of sequencing. While sequencing costs have decreased, the throughput and costs of library preparation can be a limiting factor. One aspect of library preparation includes modification of the ends of nucleic acid fragments such that they are suitable for a particular sequencing platform. Nucleic acid ends may contain useful information. Accordingly, methods that modify nucleic acid ends (e.g., for library preparation) while preserving the information contained in the nucleic acid ends would be useful for processing and analyzing nucleic acid. Nucleic acid fragment lengths also may contain useful information. Accordingly, methods that modify nucleic acid ends (e.g., for library preparation) while preserving the native nucleic acid fragment lengths would be useful for processing and analyzing nucleic acid.

Another aspect of library preparation includes capturing single stranded nucleic acid fragments. In certain instances, single-stranded library preparation methods can generate better and more complex libraries compared to traditional double-stranded DNA (dsDNA) preparation methods. Drawbacks to producing single-stranded DNA (ssDNA) libraries include labor intensive, expensive, and time-consuming protocols, and exotic or custom reagent requirements. Accordingly, methods that capture single-stranded nucleic acids (e.g., for library preparation), without requiring labor intensive, expensive, and time-consuming protocols, and/or exotic or custom reagents would be useful for processing and analyzing nucleic acid (e.g., single-stranded nucleic acid, denatured double-stranded nucleic acid, or mixtures containing single-stranded nucleic acid).

In certain applications, a library is generated from cell-free nucleic acid (e.g., cell-free DNA). Cell-free DNA (cfDNA), which can be found circulating in blood, for example, originates predominantly from dying cells. In healthy individuals the vast majority of cfDNA derives from hematopoietic myeloid and lymph cells undergoing apoptosis. However, in individuals with cancer, a variable fraction of the cfDNA derives from tumor cells undergoing apoptosis and/or necrosis. This tumor derived fraction of cfDNA is known as circulating tumor DNA (ctDNA). The amount of ctDNA found in an individual with cancer depends on numerous parameters such as tumor growth rate, metastasis, and overall tumor size.

Detection and serial monitoring of ctDNA in cancer patients, through Next-Generation Sequencing (NGS), may be useful for assessing disease progression, response to treatment, and early detection of tumors. However, the depth of sequencing necessary to detect ctDNA across the whole genome with high sensitivity and low error rates is generally cost-prohibitive. For this reason, cfDNA researchers may employ some variation of a panel enrichment procedure. For example, a NGS library generated from a patient's cfDNA may be followed by targeted enrichment of a known panel of variants that are of pathological significance in various cancers. The NGS library prep typically captures the breadth of molecules present in the cfDNA and the targeted panel can enrich for the small genomic fraction of the genome that is of clinical interest. Enriched cfDNA libraries can be cost-effectively sequenced to high depths in order to observe extremely low allele fraction of ctDNA that may reside within cfDNA.

Target-enrichment requires high depth of sequencing driving assay costs. In certain applications, cfDNA fragmentomics approaches may be useful for liquid-biopsy based cancer diagnostics. For example, a cfDNA fragment ratio can be indicative of underlying cancerous state, and a cancer-predictive signal may be obtained with a very low depth of sequencing.

A fast, simple, and efficient ligation-based single-stranded DNA library preparation (single-stranded (ss) prep) method optimized for cfDNA analysis is described herein. When coupled with downstream target-enrichment approaches, the ss prep method described herein allows for uniform enrichment and increased sequencing efficiency of targeted regions. As a single-stranded approach, the ss prep method described herein does not require additional steps for end-repair and retains a variety of cfDNA fragments often lost when traditional library prep methods are used. This results in more complex libraries that retain their native termini. These features make the ss prep approach described herein ideal for cfDNA fragmentomic analyses.

### SUMMARY

Provided in certain aspects are methods for analyzing nucleic acid fragment length in a test sample, comprising a) producing a nucleic acid library from the test sample; b) sequencing the nucleic acid library, thereby generating sequence reads; c) determining nucleic acid fragment lengths from the sequence reads; and d) analyzing the nucleic acid fragment lengths.

Also provided in certain aspects are methods for detecting the presence or absence of a population of cells in a subject comprising a) determining lengths of nucleic acid fragments in a test sample from a subject; and b) detecting the presence or absence of a population of cells in a subject according the nucleic acid fragment lengths determined in (a).

Certain implementations are described further in the following description, examples and claims, and in the drawings.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIG. 1 shows a single-stranded library prep schematic.

FIG. 2 shows a targeted enrichment workflow.

FIG. 3A shows preseq library size estimation indicating that libraries generated with the single-stranded library prep method described herein had higher complexity compared to those generated with a double-stranded prep method. FIG. 3B shows for the same amount of input, the single-stranded library prep method described herein had improved mean-target coverage compared to a double-stranded prep method.

FIG. 4 shows targeted enrichment metrics of libraries generated either with the single-stranded library prep method described or a double-stranded prep method.

FIGS. 5A and 5B show identification of KRAS G12D mutation in longitudinal plasma samples obtained from two AML progressors. Targeted enrichment was performed on longitudinal plasma samples obtained from two individuals that were initially diagnosed for MDS (01) and subsequently progressed to AML (02). FIG. 5A shows IGV browser showing the presence of KRAS G12D mutation in cfDNA from an individual with AML. FIG. 5B shows variant allele fraction of KRAS G12D mutation at time points 01 and 02.

FIG. 6 shows a fragment length analysis.

FIG. 7A shows genomic fragment length ratio profiles for healthy and MDS samples. For each sample, the GC-corrected log-ratio of short-to-long fragment counts across the genome was plotted. FIG. 7B shows comparison of each sample's correlation to the average MDS profile and the average healthy profile.

FIG. 8 shows A/B segment (open/closed chromatin compartments; active/inactive chromatin compartments) definitions.

FIG. 9 shows correlations between fragment length ratios (y-axis) and eigenvalues (x-axis) for combined DELFI samples and a single sample.

FIG. 10 shows correlations between fragment length ratios (y-axis) and distance to boundary (x-axis) for combined DELFI samples and a single sample.

FIG. 11 shows correlations between fragment length ratios (y-axis) and eigenvalues (x-axis) for combined DELFI samples, a single sample, and combined samples prepared using a single-stranded library prep method described herein (SRSLY).

FIG. 12 shows correlations between fragment length ratios (y-axis) and distance to boundary (x-axis) for combined DELFI samples, a single sample, and combined samples prepared using a single-stranded library prep method described herein (SRSLY).

FIG. 13 shows correlations between fragment length ratios (y-axis) and distance to boundary (x-axis) for combined DELFI samples, combined samples prepared using a single-stranded library prep method described herein (SRSLY), and combined MDS samples.

FIG. 14 shows correlations between fragment length ratios (y-axis) and distance to boundary (x-axis) for combined samples prepared using a single-stranded library prep method described herein (SRSLY), combined MDS samples, and combined GI samples.

FIG. 15 shows individual transitions in a sample.

FIG. 16 shows shift measurements in one sample.

FIG. 17 shows shift measurements in multiple DELFI samples.

FIG. 18 shows shift in lower coverage samples prepared using a single-stranded library prep method described herein (SRSLY).

FIG. 19 shows different sets have similar good transitions.

FIG. 20 shows 5′ and 3′ end profile motifs for A/B compartment fragments. The open (A) compartment shows weaker sequence logos compared to the closed (B) compartment (as measured by number of bits on the y-axis).

FIG. 21 shows an illustration of a eukaryotic cell with open (A) and closed (B) chromosome compartments.

FIG. 22 shows cfDNA fragment length distribution for open (A) and closed (B) chromosome compartments. Using A/B definitions from a lymphocyte cell line, the open compartment has shorter cfDNA fragments.

FIG. 23 shows results of deep sequencing. Consistent differences (shown by vertical lines) of fragment lengths were found for the RS-MLD subtype of MDS. These were present in treatment-naïve and treated samples. MDS-RS-MLD is a type of myelodysplastic syndrome (MDS) characterized by multiple lineage dysplasia (multiple types of blood cell are affected) and the presence of ring sideroblasts (early red blood cells in the bone marrow that have a ring of iron in them). MDS, myelodysplastic syndrome; RS, ring sideroblasts; MLD, multiple lineage dysplasia FIG. 24 shows a ring sideroblast classifier. With only 5 million sequencing reads, MDS-RS-MLD samples (circled) can be distinguished from healthy and other MDS samples, based on the A/B compartment definitions calculated according to cfDNA fragment length as discussed herein. MDS, myelodysplastic syndrome; RS, ring sideroblasts; MLD, multiple lineage dysplasia FIG. 25 shows difference in fragment lengths between open and closed compartments in one sample. A histogram of DNA template lengths was generated separately for both the open and closed compartments. The ratio of proportion of closed fragments to open fragments is plotted for each template length. Template lengths between approximately 140 bp and 220 bp are more prevalent in the closed compartment. Compartment definitions were taken from the GM12878 cell line from Rao et al., 2014 Cell 159(7):1665-80.

FIGS. 26A and 26B show fragment ratios of medium fragment count (100 bp to 149 bp) to long fragment count (150 bp to 219 bp) in each 250 kb window of the genome. Each genomic window's fragment count ratio is put on the x-axis in distance to the nearest open-close compartment boundary. The curve line shows the LOESS regression across all points. The upper horizontal line is the average ratio in the open compartment, and the lower horizontal line is the average ratio in the closed compartment. FIG. 26A: for each genomic window, the numerator and denominator are each the sum across all healthy samples in the cohort. FIG. 26B: for each genomic window, the numerator and denominator are the sum across all samples with an MDS diagnosis in the cohort.

FIG. 27 shows the deviation of each atypical MDS sample from the average healthy sample. Each point is a different genomic window's fragment ratio (see description of FIGS. 26A and 26B). The x-axis shows the difference in log of the fragment ratios between atypical MDS sample 1 and the average healthy sample, and the y-axis shows the difference in log of the fragment ratios in atypical MDS sample 2 and the healthy average sample.

FIG. 28 shows the compartment-oriented view of fragment length ratios for the average healthy sample, atypical MDS sample 1 (SR2345), and atypical MDS sample 2 (SR2371). See description of FIGS. 26A and 26B for a description of the plot.

FIG. 29 shows all samples with low-coverage whole genome sequencing that displayed a similar pattern to atypical MDS sample 1 and atypical MDS sample 2. Some patients have multiple samples in the table. The clinical diagnosis for most samples is MDS with subtype of RS-MLD, TDS with multiple lineage dysplasia and ring sideroblasts.

FIG. 30 shows the entire cohort along several metrics of atypical fragment length. The “open_closed” metric represents the overall difference between open and closed compartments. The “opening_closed” represents the difference between the closed compartment and the genomic regions selected from atypical MDS 1 and 2 that appeared most different. The “opening_open” metric is a difference between the open compartment and these “opening” regions. The “mds_weird” samples are those in FIG. 29, and the “not_weird” correspond to all other samples.

FIG. 31 shows a flow chart for a test described herein.

FIG. 32 shows a flow chart for MDS classification.

FIGS. 33A and 33B show fragment length ratios according to genomic compartment (see description of FIGS. 26A and 26B). In addition to the MDS and healthy cohorts, a cohort of gastrointestinal cancer cfDNA (gi) and acute-myeloid leukemia cfDNA (aml) are shown.

FIGS. 34A and 34B show the boxplot representation of fragment length ratios according to genomic compartment. This is the same data as shown in FIG. 33, but point clouds are summarized as boxplots instead.

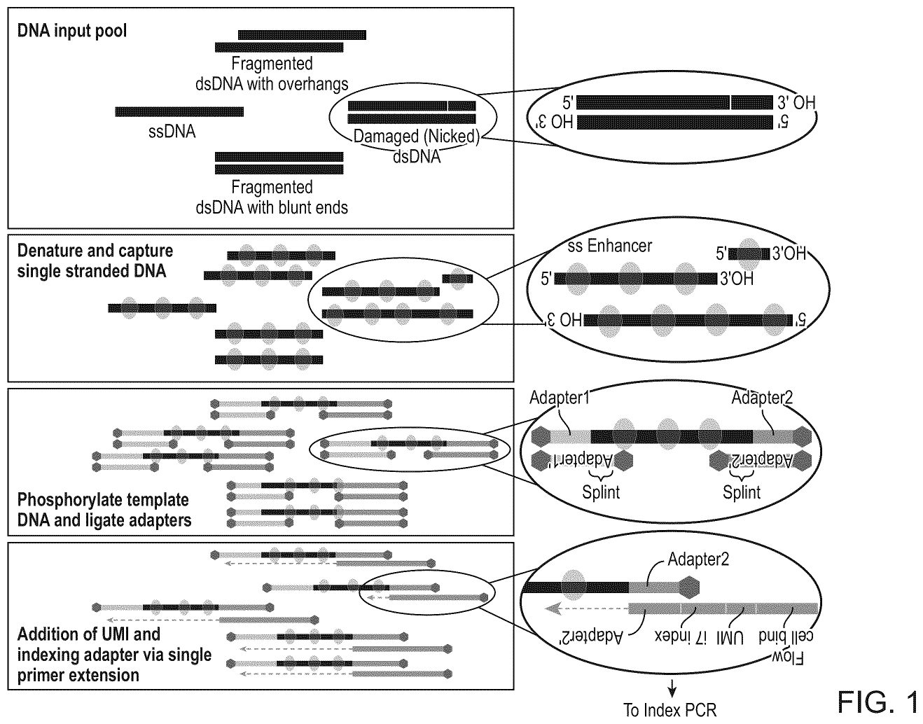

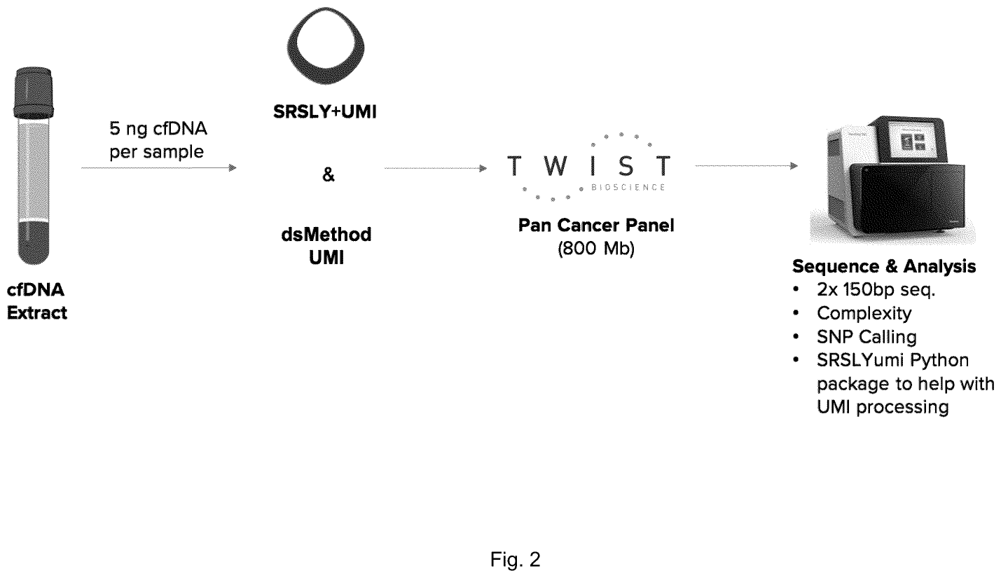

### DETAILED DESCRIPTION

Provided herein are methods and compositions useful for analyzing nucleic acid. Also provided herein are methods and compositions useful for producing nucleic acid libraries. Also provided herein are methods and compositions useful for analyzing single-stranded nucleic acid fragments. In certain aspects, the methods include combining sample nucleic acid comprising single-stranded nucleic acid fragments and specialized adapters. In some embodiments, the specialized adapters include a unique molecular identifier (UMI). In some embodiments, the specialized adapters include a scaffold polynucleotide capable of hybridizing to an end of a single-stranded nucleic acid. Products of such hybridization may be useful for producing a nucleic acid library and/or further analysis or processing, for example. In certain applications, nucleic acid fragment length is analyzed. A fragment length analysis may be useful for identifying A/B transitions in a genome. A fragment length analysis may be useful for identifying one or more genomic regions as in an A compartment or in a B compartment. A fragment length analysis may be useful for a diagnostic process (e.g., a cancer diagnosis).

Fragment Analysis

Provided herein are methods and compositions for analyzing nucleic acid fragments in a sample from a subject. In some embodiments, a method comprises detecting the presence or absence of a population of cells in a subject according to a nucleic acid fragment analysis. In some embodiments, a method herein comprises determining lengths of nucleic acid fragments (e.g., in a test sample from a subject). In some embodiments, the presence or absence of a population of cells in a subject is determined according lengths of nucleic acid fragments in a sample from the subject. Nucleic acid fragment lengths may be determined or measured by any suitable method including, but no limited to, methods described herein (e.g., single read length measurements, mapped paired-end read measurements, capillary electrophoresis). Nucleic acid fragment lengths may be categorized as long fragments or short fragments; or as long fragments, medium fragments, or short fragments. Short fragments generally refer to fragments that are about 0 bp to about 99 bp in length. Medium fragments generally refer to fragments that are about 100 bp to about 149 bp in length. Long fragments generally refer to fragments that are about 150 bp to about 220 bp in length.

In some embodiments, a method herein comprises generating one or more fragment counts. The term fragment counts generally refers to a quantification of nucleic acid fragments of a given length or range of lengths. For example, a fragment count may refer to a quantification of short nucleic acid fragments (fragments that are about 0 bp to about 99 bp in length), medium nucleic acid fragments (fragments that are about 100 bp to about 149 bp in length), and/or long fragments (fragments that are about 150 bp to about 220 bp in length). In certain instances, a fragment count may refer to a quantification of fragments at each length (e.g., each length between 1 bp to 220 bp). A quantification may be any of a single count (e.g., for a single test sample); an average, mean, or median count (e.g., for multiple test samples); a normalized count (a count adjusted for certain biases), and the like. In some embodiments, a fragment count is associated with a measure of uncertainty (e.g., standard deviation, median absolute deviation). In some embodiments, the presence or absence of a population of cells in a subject is determined according to one or more fragment counts. In some embodiments, a fragment length profile (e.g., a distribution of fragment length quantifications) is determined for a test sample. In some embodiments, the presence or absence of a population of cells in a subject is determined according to a fragment length profile.

In some embodiments, a method herein comprises generating one or more fragment length ratios. Each of the one or more fragment length ratios may be a ratio chosen from short fragment counts to long fragment counts, short fragment counts to medium fragment counts, and medium fragment counts to long fragment counts. One or more fragment length ratios may be generated for one or more genomic portions. In some embodiments, the one or more genomic portions are about 50 kb to about 500 kb in length. For example, a genomic portion may be about 50 kb in length, about 100 kb in length, about 150 kb in length, about 200 kb in length, about 250 kb in length, about 300 kb in length, about 350 kb in length, about 400 kb in length, about 450 kb in length, or about 500 kb in length. In some embodiments, a genomic portion is about 100 kb in length. In some embodiments, a genomic portion is about 250 kb in length. A fragment length ratio may be any of a single ratio (e.g., for a single test sample; for a single genomic portion); an average, mean, or median ratio (e.g., for multiple test samples; for multiple genomic portions; for multiple ratios for a test sample; for multiple ratios for a genomic portion); a normalized ratio (a ratio adjusted for certain biases), and the like. In some embodiments, a fragment length ratio is associated with a measure of uncertainty (e.g., standard deviation, median absolute deviation). In some embodiments, the presence or absence of a population of cells in a subject is detected according to the one or more fragment length ratios. In some embodiments, a fragment length ratio profile (e.g., a distribution of fragment length ratios (e.g., over a plurality of genomic portions)) is determined for a test sample. In some embodiments, the presence or absence of a population of cells in a subject is determined according to a fragment length ratio profile.

In some embodiments, a method herein comprises comparing nucleic acid fragment lengths, or a derivative thereof, for a test sample to nucleic acid fragment lengths, or a derivative thereof, for one or more control samples, thereby generating a comparison. In some embodiments, detecting the presence or absence of a population of cells in a subject is according to the comparison. A derivative of fragments lengths generally refers to any suitable fragment length quantification, ratio, or profile (e.g., fragment length quantifications, fragment length ratios, fragment length profiles, and/or fragment length ratio profiles described herein). A comparison may include one or more of a visual inspection (e.g., of a test profile compared to a control profile), a mathematical analysis, a statistical analysis, a regression analysis, and the like. In certain embodiments, a comparison comprises generating one or more values. Non-limiting examples of values that can be generated in a comparison include a sensitivity, specificity, standard deviation, median absolute deviation (MAD), measure of certainty, measure of confidence, measure of certainty or confidence that a value obtained for a test sample is inside or outside a particular range of values, measure of uncertainty, measure of uncertainty that a value obtained for a test sample is inside or outside a particular range of values, coefficient of variation (CV), confidence level, confidence interval (e.g., about 95% confidence interval), standard score (e.g., z-score), chi value, phi value, result of a t-test, p-value, area ratio, median level, the like or combination thereof. In some embodiments, a comparison comprises a t-test.

A control sample may be a disease sample or a non-disease sample. In some embodiments, a control sample is obtained from a healthy subject. In some embodiments, a control sample is obtained from a diseased subject. In some embodiments, a control sample is obtained from healthy tissue. In some embodiments, a control sample is obtained from diseased tissue. A disease sample generally is from a portion of a tissue of an organism or subject identified as being diseased, and a non-disease sample generally is from a portion of a tissue of an organism or subject identified as not being diseased. A disease sample and a non-disease sample sometimes are from the same subject, and sometimes are not from the same subject. A disease sample sometimes is from a portion of a tissue of an organism identified as being diseased, and a non-disease sample sometimes is from a portion of tissue of an organism identified as not being diseased. For example, a disease sample sometimes is from a cancer tumor and a non-disease sample sometimes is from a non-tumor tissue in the same subject. A disease may be is a condition, and a disease or condition sometimes is diagnosed, inferred or suspected for a subject. Non-limiting examples of disease samples include samples from subjects having or suspected of having cancer, kidney disease, bladder disease, a neurodegenerative disease, and subtypes thereof. A disease sample may be a cancer sample, and non-limiting examples of cancer samples include samples from subjects having or suspected of having a cancer type or subtype described herein. For example, a cancer may be myelodysplastic syndrome (MDS) and a cancer subtype may be MDS with multiple lineage dysplasia and ring sideroblasts (MDS-RS-MLD). In some embodiments, a cancer is gastrointestinal cancer. In some embodiments, a cancer is acute-myeloid leukemia.

In some embodiments, a method herein comprises enriching a test sample for one or more selected genomic regions, thereby generating one or more pools of enriched nucleic acid. A test sample may be enriched according to any suitable nucleic acid enrichment method (e.g., an enrichment method described herein). For example, an enrichment method may comprise hybridizing capture probes to nucleic acid fragments in the test sample, where the capture probes are specific to one or more selected genomic regions. In some embodiments, one or more genomic regions are selected according to A/B status. A/B status refers to open/active (“A”) chromatin compartments and closed/inactive (“B”) chromatin compartments (e.g., as described in Rao et al., 2014 Cell 159(7):1665-80), and generally is cell-type specific. A/B compartments may be mapped for a given cell type using spatial proximity analysis (e.g., 3-C, Hi-C analysis). Genomic regions in compartment A tend to interact preferentially with A compartment-associated regions than B compartment-associated ones. Similarly, regions in compartment B tend to associate with other B compartment-associated regions. A/B compartment-associated regions typically are on the multi-megabase (Mb) scale and correlate with either open and expression-active chromatin (“A” compartments) or closed and expression-inactive chromatin (“B” compartments). A compartments generally are gene-rich, have high GC-content, contain histone markers for active transcription, and are located in the interior of the nucleus. B compartments generally are gene-poor, compact, contain histone markers for gene silencing, and are located on the nuclear periphery. A/B compartments may be further divided into subcompartments.

In some embodiments, one or more genomic regions are selected for enrichment according to A/B status for a chosen cell type (e.g., a diseased cell type; a cancer cell type). In some embodiments, one or more genomic regions are selected for enrichment according to A/B status differences between a diseased cell and a non-diseased cell. In some embodiments, a test sample is enriched prior to determining lengths of nucleic acid fragments in the test sample.

In some embodiments, a method herein comprises identifying an A/B status of the genome, or portion thereof, for a test sample according to nucleic acid fragment lengths or a derivative thereof. For example, without being limited by theory, A compartments and B compartments may be associated with particular fragment length quantifications, fragment length profiles, fragment length ratios, and/or fragment length ratio profiles. Accordingly, in some embodiments, an A/B status of the genome, or portion thereof, for a test sample may be determined according to fragment length quantifications, fragment length profiles, fragment length ratios, and/or fragment length ratio profiles described herein.

In some embodiments, a method herein comprises comparing an A/B status of the genome, or portion thereof, for a test sample to an A/B status of a control genome, thereby generating an A/B status comparison. In some embodiments, detecting the presence or absence of a population of cells in a subject is according to the A/B status comparison. A comparison may include one or more of a visual inspection, a mathematical analysis, a statistical analysis, a regression analysis, and the like. In some embodiments, a comparison comprises generating one or values as described above. In some embodiments, a comparison comprises a t-test. A control genome may be from one or more non-diseased cells, or may be from one or more diseased cells, as described above.

In some embodiments, the presence or absence of a population of cells in a subject is detected according to a comparison described herein. For example, a particular cell type may have a unique fragment length signature and/or A/B status signature distinguishable from other cell types that may be detected in a sample. In some embodiments, the population of cells comprises diseased cells. In some embodiments, the population of cells comprises cancer cells. In some embodiments, the population of cells comprises diseased bladder cells. In some embodiments, the population of cells comprises diseased neuronal cells. In some embodiments, a method comprises detecting a presence or absence of a disease or a disease subtype for a subject according to the presence or absence of the population of cells.

Scaffold Adapters

Certain methods herein comprise combining single stranded nucleic acid (ssNA) with scaffold adapters, or components thereof. Scaffold adapters generally include a scaffold polynucleotide and an oligonucleotide. Accordingly, a “component” of a scaffold adapter may refer to a scaffold polynucleotide and/or an oligonucleotide, or a subcomponent or region thereof. The oligonucleotide and/or the scaffold polynucleotide can be composed of pyrimidine (C, T, U) and/or purine (A, G) nucleotides. Additional components or subcomponents may include one or more of an index polynucleotide, a unique molecular identifier (UMI), one or more regions that flank a unique molecular identifier (UMI), primer binding site (e.g., sequencing primer binding site, P5 primer binding site, P7 primer binding site), flow cell binding region, and the like, and complements thereto. Scaffold adapters comprising a P5 primer binding site may be referred to as P5 adapters or P5 scaffold adapters. Scaffold adapters comprising a P7 primer binding site may be referred to as P7 adapters or P7 scaffold adapters.

A scaffold polynucleotide is a single-stranded component of a scaffold adapter. A polynucleotide herein generally refers to a single-stranded multimer of nucleotide from 5 to 500 nucleotides, e.g., 5 to 100 nucleotides. Polynucleotides may be synthetic or may be made enzymatically, and, in some embodiments, are about 5 to 50 nucleotides in length. Polynucleotides may contain ribonucleotide monomers (i.e., may be polyribonucleotides or “RNA polynucleotides”), deoxyribonucleotide monomers (i.e., may be polydeoxyribonucleotides or “DNA polynucleotides”), or a combination thereof. Polynucleotides may be 10 to 20, 20 to 30, 30 to 40, 40 to 50, 50 to 60, 60 to 70, 70 to 80, 80 to 100, 100 to 150 or 150 to 200, or up to 500 nucleotides in length, for example. The terms polynucleotide and oligonucleotide may be used interchangeably.

A scaffold polynucleotide may include an ssNA hybridization region (also referred to as scaffold, scaffold region, single-stranded scaffold, single-stranded scaffold region) and an oligonucleotide hybridization region. An ssNA hybridization region and an oligonucleotide hybridization region may be referred to as subcomponents of a scaffold polynucleotide. An ssNA hybridization region typically comprises a polynucleotide that hybridizes, or is capable of hybridizing, to an ssNA terminal region. An oligonucleotide hybridization region typically comprises a polynucleotide that hybridizes, or is capable of hybridizing, to all or a portion of the oligonucleotide component of the scaffold adapter.

An ssNA hybridization region of a scaffold polynucleotide may comprise a polynucleotide that is complementary, or substantially complementary, to an ssNA terminal region (e.g., an ssDNA terminal region, an sscDNA terminal region, an ssRNA terminal region). In some embodiments, an ssNA hybridization region is an ssDNA hybridization region, an sscDNA hybridization region, or an ssRNA hybridization region. In some embodiments, an ssNA hybridization region comprises a random sequence. In some embodiments, an ssNA hybridization region comprises a sequence complementary to an ssNA terminal region sequence of interest (e.g., targeted sequence). In certain embodiments, an ssNA hybridization region comprises one or more nucleotides that are all capable of non-specific base pairing to bases in the ssNA. Nucleotides capable of non-specific base pairing may be referred to as universal bases. A universal base is a base capable of indiscriminately base pairing with each of the four standard nucleotide bases: A, C, G and T. Universal bases that may be incorporated into the ssNA hybridization region include, but are not limited to, inosine, deoxyinosine, 2′-deoxyinosine (dl, dlnosine), nitroindole, 5-nitroindole, and 3-nitropyrrole. In certain embodiments, an ssNA hybridization region comprises one or more degenerate/wobble bases which can replace two or three (but not all) of the four typical bases (e.g., non-natural base P and K).

An ssNA hybridization region of a scaffold polynucleotide may have any suitable length and sequence. In some embodiments, the length of the ssNA hybridization region is 10 nucleotides or less. In certain aspects, the ssNA hybridization region is from 4 to 100 nucleotides in length, e.g., about 5, 10, 15, 20, 25, 30, 35, 40, 45, 50, 55, 60, 65, 70, 75, 80, 85, 90, 95, or 100 nucleotides in length. In certain aspects, the ssNA hybridization region is from 4 to 20 nucleotides in length, e.g., from 5 to 15, 5 to 10, 5 to 9, 5 to 8, or 5 to 7 (e.g., 6 or 7) nucleotides in length. In some embodiments, the ssNA hybridization region is 7 nucleotides in length. In some embodiments, the ssNA hybridization region comprises or consists of a random nucleotide sequence, such that when a plurality of heterogeneous scaffold polynucleotides having various random ssNA hybridization regions are employed, the collection is capable of acting as scaffold polynucleotides for a heterogeneous population of ssNAs irrespective of the sequences of the terminal regions of the ssNAs. Each scaffold polynucleotide having a unique ssNA hybridization region sequence may be referred to as a scaffold polynucleotide species and a collection of multiple scaffold polynucleotide species may be referred to as a plurality of scaffold polynucleotide species (e.g., for a scaffold polynucleotide designed to have 7 random bases in the ssNA hybridization region, a plurality of scaffold polynucleotide species would include 4⁷ unique ssNA hybridization region sequences). Accordingly, each scaffold adapter having a unique scaffold polynucleotide (i.e., comprising a unique ssNA hybridization region sequence) may be referred to as a scaffold adapter species and a collection of multiple scaffold adapter species may be referred to as a plurality of scaffold adapter species. A species of scaffold polynucleotide generally contains a feature that is unique with respect to other scaffold polynucleotide species. For example, a scaffold polynucleotide species may contain a unique sequence feature. A unique sequence feature may include a unique sequence length, a unique nucleotide sequence (e.g., a unique random sequence, a unique targeted sequence), or a combination of a unique sequence length and nucleotide sequence.

A scaffold polynucleotide may comprise one or more additional subcomponents including an index polynucleotide, a unique molecular identifier (UMI), one or more regions that flank a unique molecular identifier (UMI), primer binding site (e.g., P5 primer binding site, P7 primer binding site), flow cell binding region, and the like, or complementary polynucleotides thereof. A scaffold polynucleotide may comprise a primer binding site (or a polynucleotide complementary to a primer binding site). Scaffold polynucleotides comprising a P5 primer binding site (or complement thereof) may be referred to as P5 scaffolds or P5 scaffold polynucleotides. Scaffold polynucleotides comprising a P7 primer binding site (or complement thereof) may be referred to as P7 scaffolds or P7 scaffold polynucleotides.

An oligonucleotide can be a further single-stranded component of a scaffold adapter. An oligonucleotide herein generally refers to a single-stranded multimer of nucleotides from 5 to 500 nucleotides, e.g., 5 to 100 nucleotides. Oligonucleotides may be synthetic or may be made enzymatically, and, in some embodiments, are 5 to 50 nucleotides in length. Oligonucleotides may contain ribonucleotide monomers (i.e., may be oligoribonucleotides or “RNA oligonucleotides”), deoxyribonucleotide monomers (i.e., may be oligodeoxyribonucleotides or “DNA oligonucleotides”), or a combination thereof. Oligonucleotides may be 10 to 20, 20 to 30, 30 to 40, 40 to 50, 50 to 60, 60 to 70, 70 to 80, 80 to 100, 100 to 150 or 150 to 200, or up to 500 nucleotides in length, for example. The terms oligonucleotide and polynucleotide may be used interchangeably.

An oligonucleotide component of a scaffold adapter generally comprises a nucleic acid sequence that is complementary or substantially complementary to an oligonucleotide hybridization region of a scaffold polynucleotide. An oligonucleotide component of a scaffold adapter may include one or more subcomponents useful for one or more downstream applications such as, for example, PCR amplification of the ssNA fragment or derivative thereof, sequencing of the ssNA or derivative thereof, and the like. In some embodiments, a subcomponent of an oligonucleotide is a sequencing adapter. Sequencing adapter generally refers to one or more nucleic acid domains that include at least a portion of a nucleotide sequence (or complement thereof) utilized by a sequencing platform of interest, such as a sequencing platform provided by Illumina® (e.g., the HiSeq™, MiSeq™ and/or Genome Analyzer™ sequencing systems); Oxford Nanopore™ Technologies (e.g., the MinION™ sequencing system), Ion Torrent™ (e.g., the Ion PGM™ and/or Ion Proton™ sequencing systems); Pacific Biosciences (e.g., a Sequel or PACBIO RS II sequencing system); Life Technologies™ (e.g., a SOLiD™ sequencing system); Roche (e.g., the 454 GS FLX+ and/or GS Junior sequencing systems); Genapsys; BGI; or any sequencing platform of interest.

In some embodiments, an oligonucleotide component of a scaffold adapter is, or comprises, a nucleic acid domain selected from: a domain (e.g., a “capture site” or “capture sequence”) that specifically binds to a surface-attached sequencing platform oligonucleotide (e.g., a P5 or P7 oligonucleotide attached to the surface of a flow cell in an Illumina® sequencing system); a sequencing primer binding domain (e.g., a domain to which the Read 1 or Read 2 primers of the Illumina® platform may bind); a unique identifier or index (e.g., a barcode or other domain that uniquely identifies the sample source of the ssNA being sequenced to enable sample multiplexing by marking every molecule from a given sample with a specific barcode or “tag”); a barcode sequencing primer binding domain (a domain to which a primer used for sequencing a barcode binds); a molecular identification domain or unique molecular identifier (UMI) (e.g., a molecular index tag, such as a randomized tag of 4, 6, or other number of nucleotides) for uniquely marking molecules of interest, e.g., to determine expression levels based on the number of instances a unique tag is sequenced; a complement of any such domains; or any combination thereof. In some embodiments an oligonucleotide comprises one or more regions that flank a unique molecular identifier (UMI). In some embodiments, a barcode domain (e.g., sample index tag) and a molecular identification domain (e.g., a molecular index tag; UMI) may be included in the same nucleic acid. Sequencing platform oligonucleotides, sequencing primers, and their corresponding binding domains can be designed to be compatible with a variety of available sequencing platforms and technologies, including but not limited to those discussed herein.

When an oligonucleotide component of a scaffold adapter includes one or a portion of a sequencing adapter, one or more additional sequencing adapters and/or a remaining portion of the sequencing adapter may be added using a variety of approaches. For example, additional and/or remaining portions of sequencing adapters may be added by any one of ligation, reverse transcription, PCR amplification, and the like. In the case of PCR, an amplification primer pair may be employed that includes a first amplification primer that includes a 3′ hybridization region (e.g., for hybridizing to an adapter region of the oligonucleotide) and a 5′ region including an additional and/or remaining portion of a sequencing adapter, and a second amplification primer that includes a 3′ hybridization region (e.g., for hybridizing to an adapter region of a second oligonucleotide added to the opposite end of an ssNA molecule) and optionally a 5′ region including an additional and/or remaining portion of a sequencing adapter.

An oligonucleotide component of a scaffold adapter may comprise one or more additional subcomponents including an index polynucleotide, a unique molecular identifier (UMI), one or more regions that flank a unique molecular identifier (UMI), primer binding site (e.g., P5 primer binding site, P7 primer binding site), flow cell binding region or sequencing adapter, and the like, or complementary polynucleotides thereof. An oligonucleotide may comprise a primer binding site (or a polynucleotide complementary to a primer binding site). Oligonucleotides comprising a P5 primer binding site (or complement thereof) may be referred to as P5 oligos or P5 oligonucleotides. Oligonucleotides comprising a P7 primer binding site (or complement thereof) may be referred to as P7 oligos or P7 oligonucleotides.

An oligonucleotide component of a scaffold adapter may comprise a guanine and cytosine (GC)-rich region. A GC-rich region may comprise at least about 50% guanine and cytosine nucleotides. For example, a GC-rich region may comprise about 60% guanine and cytosine nucleotides, about 70% guanine and cytosine nucleotides, about 80% guanine and cytosine nucleotides, about 90% guanine and cytosine nucleotides, or 100% guanine and cytosine nucleotides. In some embodiments, a GC-rich region comprises about 70% guanine and cytosine nucleotides. An oligonucleotide component of a scaffold adapter may comprise a guanine and cytosine (GC)-rich region at one end (e.g., at a 3′ end or at a 5′ end). In some embodiments, an oligonucleotide component of a scaffold adapter comprises a guanine and cytosine (GC)-rich region at the end of the oligonucleotide that is joined to an ssNA fragment (i.e., at the oligonucleotide-ssNA junction or “ligation terminus”). A scaffold polynucleotide may comprise a corresponding region that is complementary to the GC-rich region in the oligonucleotide.

The scaffold polynucleotide may be hybridized to the oligonucleotide, forming a duplex in the scaffold adapter. Accordingly, a scaffold adapter may be referred to as a scaffold duplex, a duplex adapter, a duplex oligonucleotide, or a duplex polynucleotide. Each scaffold duplex having a unique scaffold polynucleotide (i.e., comprising a unique ssNA hybridization region sequence) may be referred to as a scaffold duplex species and a collection of multiple scaffold duplex species may be referred to as a plurality of scaffold duplex species. In some embodiments, the scaffold polynucleotide and the oligonucleotide are on separate DNA strands. In some embodiments, the scaffold polynucleotide and the oligonucleotide are on a single DNA strand (e.g., a single DNA strand capable of forming a hairpin structure).

Scaffold adapters can comprise DNA, RNA, or a combination thereof. Scaffold adapters can comprise a DNA scaffold polynucleotide and a DNA oligonucleotide, a DNA scaffold polynucleotide and an RNA oligonucleotide, an RNA scaffold polynucleotide and a DNA oligonucleotide, or an RNA scaffold polynucleotide and an RNA oligonucleotide. In one example configuration, a scaffold adapter comprises a DNA scaffold polynucleotide and a DNA oligonucleotide for combining with an RNA sample nucleic acid, and example ligases for use with such an adapter/sample configuration include T4 RNA ligase 2 and T4 DNA ligase. In another example adapter configuration, a scaffold adapter comprises a DNA scaffold polynucleotide and an RNA oligonucleotide for combining with an RNA sample nucleic acid, and example ligases for use with such an adapter/sample configuration include T4 RNA ligase 1. In another example adapter configuration, a scaffold adapter comprises an RNA scaffold polynucleotide and an RNA oligonucleotide for combining with an RNA sample nucleic acid, and example ligases for use with such an adapter/sample configuration include T4 RNA ligase 1. In some instances, the adapter nucleotide composition is selected to provide homogeneity between sample nucleic acids and scaffold adapter nucleic acids (e.g., such that at least the oligonucleotide is homogenous to the sample nucleic acids). In some instances, the adapter nucleotide composition is selected to provide homogeneity between the oligonucleotide and the sample nucleic acids and heterogeneity between the scaffold polynucleotide and the sample nucleic acids.

Unique Molecular Identifier (UMI)

In some embodiments, a scaffold adapter comprises a unique molecular identifier (UMI). In some embodiments, an oligonucleotide (e.g., an oligonucleotide component of a scaffold adapter) comprises a unique molecular identifier (UMI). Unique molecular identifiers (UMIs), which also may be referred to as molecular barcodes, barcodes, molecular identification domains, molecular index tags, sequence tags, and/or tags, generally are short sequences (e.g., about 3 to about 10 nucleotides in length) that may be added to nucleic acid fragments during nucleic acid library preparation to identify or mark input nucleic acid molecule(s). In certain applications, UMIs may be useful for uniquely marking molecules of interest, e.g., to determine expression levels based on the number of instances a unique tag is sequenced. UMIs typically are added prior to an amplification step (e.g., PCR amplification), and may be useful for reducing errors and quantitative bias introduced by amplification, for example. Scaffold adapters and/or oligonucleotide components of scaffold adapters comprising a UMI as described herein may be referred to as comprising an “in-line” UMI. An in-line UMI generally refers to a UMI sequence that is a component a scaffold adapter and/or an oligonucleotide described herein that becomes part of the sequence read generated by the sequencing of an ssNA fragment ligated to an oligonucleotide component of the scaffold adapter. When a scaffold adapter comprises an in-line UMI, library generation may not require certain additional processing steps (e.g., addition of a UMI to the adapter by way of an extension step using a strand displacing polymerase).

In some embodiments, a UMI comprises a random sequence. In some embodiments, a UMI comprises a nonrandom sequence. In some embodiments, a UMI comprises one or more universal bases. In some embodiments, a UMI consists of a random sequence. In some embodiments, a UMI consists of a nonrandom sequence. In some embodiments, a UMI consists of universal bases. A UMI may be of any suitable length. In some embodiments, a UMI comprises between three to ten nucleotides. For example, a UMI may comprise three nucleotides, four nucleotides, five nucleotides, six nucleotides, seven nucleotides, eight nucleotides, nine nucleotides, or ten nucleotides. In some embodiments, a UMI comprises five nucleotides. In some embodiments, a UMI comprises five random nucleotides. In some embodiments, a UMI comprises five nonrandom nucleotides. In some embodiments, a UMI comprises five universal bases.

In some embodiments, an oligonucleotide (e.g., an oligonucleotide component of a scaffold adapter) comprises a unique molecular identifier (UMI) flanked by one or two flank regions. A UMI flanked by a flank region is typically adjacent to the flank region. A UMI flanked by two flank regions is typically adjacent to each flank region, where the UMI is located between the two flank regions. A flank region, also referred to as an anchor sequence, may be located at an oligonucleotide end that is adjacent to the ssNA terminus, when a complex is formed (i.e., adjacent to the oligonucleotide-ssNA junction or “ligation terminus”). A flank region generally comprises a nonrandom sequence. In some embodiments, a flank region comprises a nonrandom sequence species from a pool of nonrandom sequence species. In some embodiments, a pool of nonrandom sequence species comprises two or more nonrandom sequence species. In some embodiments, a pool of nonrandom sequence species comprises three or more nonrandom sequence species. In some embodiments, a pool of nonrandom sequence species comprises four or more nonrandom sequence species. In some embodiments, a pool of nonrandom sequence species comprises five or more nonrandom sequence species. In some embodiments, a pool of nonrandom sequence species comprises six or more nonrandom sequence species. In some embodiments, a pool of nonrandom sequence species comprises four nonrandom sequence species. A flank region may be of any suitable length. In some embodiments, a flank region comprises between eight to fifteen nucleotides. For example, a flank region may comprise eight nucleotides, nine nucleotides, ten nucleotides, eleven nucleotides, twelve nucleotides, thirteen nucleotides, fourteen nucleotides, fifteen nucleotides, sixteen nucleotides, seventeen nucleotides, eighteen nucleotides, nineteen nucleotides, or twenty nucleotides. In some embodiments, a flank region comprises ten nucleotides. The combination of a UMI sequence (e.g., five random bases) and a particular flank sequence species (e.g., ten nonrandom bases from a pool of four possible flank sequence species) may serve as a molecular identifier and may be considered a “UMI.”

A flank region may be designed to have a suitable melting temperature (Tm). As described herein, melting temperature generally refers to the temperature at which half of the flank regions/polynucleotides complementary to the flank regions remain hybridized and half of the flank regions/polynucleotides complementary to the flank regions dissociate into single strands. A suitable melting temperature may be a temperature that is higher than the temperature at which a ligation reaction is performed (e.g., a ligation reaction described herein). For example, if a ligation reaction is performed at 37° C., then a suitable melting temperature for a flank region is a temperature greater than 37° C. If a ligation reaction is performed at 16° C., then a suitable melting temperature is a temperature greater than 16° C. In some embodiments, a suitable melting temperature is equal to or greater than about 37° C. For example, a suitable melting temperature may be equal to or greater than about 38° C., 39° C., 40° C., 41° C., 42° C., 43° C., 44° C., 45° C., 46° C., 47° C., 48° C., 49° C., or 50° C. In some embodiments, a suitable melting temperature is equal to or greater than about 38° C. In some embodiments, a suitable melting temperature is equal to or greater than about 45° C.

In certain configurations, a flank region may be designed to be of sufficient length, to have sufficient guanine and cytosine content, and/or comprise one or more modified nucleotides (e.g., locked nucleic acid (LNA) bases) to have a suitable melting temperature (Tm). Generally, increasing flank region length may compensate for lower GC content, and increasing GC content may compensate for shorter flank regions (i.e., provide a flank region with a suitable Tm). For example, a flank region may comprise ten nucleotides where 70% of the nucleotides are guanine or cytosine for a Tm that is greater than 45° C. In another example, a flank region may comprise eighteen nucleotides where 50% of the nucleotides are guanine or cytosine for a Tm that is greater than 45° C. For the above examples, flank regions may be shorter and/or contain lower GC content if one or modified nucleotides that increase Tm (e.g., LNA bases) are included in the flank.

A flank region may be guanine and cytosine (GC)-rich. A GC-rich flank region may comprise at least about 50% guanine and cytosine nucleotides. For example, a GC-rich flank region may comprise about 60% guanine and cytosine nucleotides, about 70% guanine and cytosine nucleotides, about 80% guanine and cytosine nucleotides, about 90% guanine and cytosine nucleotides, or 100% guanine and cytosine nucleotides. In some embodiments, a GC-rich flank region comprises about 70% guanine and cytosine nucleotides. In some embodiments, a flank region comprises about 90% guanine and cytosine nucleotides. In some embodiments, a flank region comprises about 90% guanine and cytosine nucleotides and has a Tm of about 38° C. In some embodiments, a flank region comprises the following polynucleotide sequence: GGCCCGACGG.

An oligonucleotide may comprise a further flank region. A further flank region may be at a position that is distal to the oligonucleotide end that is adjacent to the ssNA terminus, when a complex is formed (i.e., distal to the oligonucleotide-ssNA junction or “ligation terminus”). A further flank region generally comprises a nonrandom sequence. A further flank region may comprise any of the features of a flank region or anchor sequence described herein. In some configurations, a further flank region comprises one or more additional subcomponents of the oligonucleotide component of a scaffold adapter. For example, a further flank region may comprise one or more of a primer binding domain, sequencing adapter, or part thereof, and an index (e.g., a sample identification index).

In some embodiments, an oligonucleotide comprises, in order starting from the oligonucleotide-ssNA junction end, a flank region, followed by a UMI, followed by a further flank region. In some embodiments, an oligonucleotide comprises, in order starting from the oligonucleotide-ssNA junction end, a nonrandom flank region, followed by a random UMI, followed by a further nonrandom flank region. In some embodiments, an oligonucleotide comprises, in order starting from the oligonucleotide-ssNA junction end, a nonrandom flank region, followed by a nonrandom UMI, followed by a further nonrandom flank region.

In some embodiments, a scaffold polynucleotide comprises an oligonucleotide hybridization region that comprises a polynucleotide complementary to a flank region in the oligonucleotide. In some embodiments, a scaffold polynucleotide comprises an oligonucleotide hybridization region that comprises a polynucleotide complementary to a flank region in the oligonucleotide and a polynucleotide complementary to a further flank region in the oligonucleotide. In some embodiments, a scaffold polynucleotide comprises an oligonucleotide hybridization region that comprises a region that corresponds to a UMI in the oligonucleotide. A region that corresponds to a UMI in the oligonucleotide may comprise a sequence that is complementary to the UMI or may comprise a sequence that is not complementary to the UMI. When an oligonucleotide comprises a random UMI sequence, a region that corresponds to the UMI may also comprise a random sequence, and thus the UMI and the region that corresponds to the UMI generally are not complementary. A random UMI sequence and a region that corresponds to the UMI may contain the same number of nucleotides or may contain different numbers of nucleotides. When an oligonucleotide comprises a nonrandom UMI sequence, a region that corresponds to the UMI may also comprise a nonrandom sequence, and the UMI and the region that corresponds to the UMI are designed to be complementary. When an oligonucleotide comprises a UMI comprising universal bases, a region that corresponds to the UMI may also comprise universal bases. In some embodiments, a scaffold polynucleotide comprises an oligonucleotide hybridization region that comprises a region that corresponds to a UMI in the oligonucleotide flanked by a polynucleotide complementary to a flank region in the oligonucleotide and a polynucleotide complementary to a further flank region in the oligonucleotide.

Each oligonucleotide having a unique UMI configuration (i.e., comprising a unique UMI sequence and/or a unique UMI sequence combined with a particular flank sequence species) may be referred to as an oligonucleotide species and a collection of multiple oligonucleotide species may be referred to as a plurality of oligonucleotide species (e.g., for a oligonucleotide designed to have a 5 random base UMI, a plurality of oligonucleotide species may include 4⁵ unique UMI sequences). Accordingly, each scaffold adapter having a unique oligonucleotide (i.e., comprising a unique UMI sequence and/or a unique UMI sequence combined with a particular flank sequence species) and/or a unique scaffold polynucleotide (i.e., comprising a unique ssNA hybridization region sequence) may be referred to as a scaffold adapter species and a collection of multiple scaffold adapter species may be referred to as a plurality of scaffold adapter species. A species of oligonucleotide generally contains a feature that is unique with respect to other oligonucleotide species. For example, an oligonucleotide species may contain a unique sequence feature. A unique sequence feature may include a unique sequence length, a unique nucleotide sequence (e.g., a unique random sequence), or a combination of a unique sequence length and nucleotide sequence.

Combining Scaffold Adapters, or Components Thereof, and ssNA

A method herein may comprise combining one or more scaffold adapters, or components thereof, with a composition comprising single-stranded nucleic acid (ssNA) to form one or more complexes. The scaffold polynucleotide is designed for simultaneous hybridization to an ssNA fragment and an oligonucleotide component such that, upon complex formation, an end of the oligonucleotide component is adjacent to an end of the terminal region of the ssNA fragment. Typically, upon complex formation, a 5′ end of the oligonucleotide component is adjacent to a 3′ end of the terminal region of the ssNA, or a 5′ end of the oligonucleotide component is adjacent to a 3′ end of the terminal region of the ssNA. Upon complex formation in instances where a scaffold adapter is attached to both ends of an ssNA fragment, a 5′ end of one oligonucleotide component is adjacent to a 3′ end of one terminal region of the ssNA, and a 5′ end of a second oligonucleotide component is adjacent to a 3′ end of a second terminal region of the ssNA.

In some embodiments, a method includes forming complexes by combining an ssNA composition, an oligonucleotide, and a plurality of heterogeneous scaffold polynucleotides having various random ssNA hybridization regions capable of acting as scaffolds for a heterogeneous population of ssNA having terminal regions of undetermined sequence. In some embodiments, a method includes forming complexes by combining an ssNA composition, a plurality of heterogeneous oligonucleotides having various UMI configurations, and a plurality of heterogeneous scaffold polynucleotides having various random ssNA hybridization regions capable of acting as scaffolds for a heterogeneous population of ssNA having terminal regions of undetermined sequence. In some embodiments, a method includes forming complexes by combining an ssNA composition, an oligonucleotide or a plurality of heterogeneous oligonucleotides having various UMI configurations, and a plurality of heterogeneous scaffold polynucleotides, where the scaffold polynucleotides are provided in an amount that exceeds the amount of oligonucleotides. In some embodiments, scaffold polynucleotides and oligonucleotides are provided at a ratio of at least 1.1 to 1 (scaffold polynucleotides to oligonucleotides). For example, scaffold polynucleotides and oligonucleotides may be provided at a ratio of at least 1.2 to 1, 1.3 to 1, 1.4 to 1, 1.5 to 1, 1.6 to 1, 1.7 to 1, 1.8 to 1, 1.9 to 1, or 2 to 1. In some embodiments, scaffold polynucleotides and oligonucleotides are provided at a ratio of 1.4 to 1 (scaffold polynucleotides to oligonucleotides). For example, a method may comprise combining an ssNA composition with 14 μM scaffold polynucleotides and 10 μM oligonucleotides.

In some embodiments, an ssNA hybridization region includes a known sequence designed to hybridize to an ssNA terminal region of known sequence. In some embodiments, two or more heterogeneous scaffold polynucleotides having different ssNA hybridization regions of known sequence are designed to hybridize to respective ssNA terminal regions of known sequence. Embodiments in which the ssNA hybridization regions have a known sequence may be useful, for example, for producing a nucleic acid library from a subset of ssNAs having terminal regions of known sequence. Accordingly, in certain embodiments, a method herein comprises forming complexes by combining an ssNA composition, an oligonucleotide, and one or more heterogeneous scaffold polynucleotides having one or more different ssNA hybridization regions of known sequence capable of acting as scaffolds for one or more ssNAs having one or more terminal regions of known sequence.

An ssNA fragment, an oligonucleotide, and scaffold polynucleotide may be combined in various ways. In some configurations, the combining includes combining 1) a complex comprising the scaffold polynucleotide hybridized to the oligonucleotide component via the oligonucleotide hybridization region, and 2) the ssNA fragment. In another configuration, the combining includes combining 1) a complex comprising the scaffold polynucleotide hybridized to the ssNA fragment via the ssNA hybridization region, and 2) the oligonucleotide component. In another configuration, the combining includes combining 1) the ssNA fragment, 2) the oligonucleotide, and 3) the scaffold polynucleotide, where none of the three components are pre-complexed with, or hybridized to, another component prior to the combining.

The combining may be carried out under hybridization conditions such that complexes form including a scaffold polynucleotide hybridized to a terminal region of an ssNA fragment via the ssNA hybridization region, and the scaffold polynucleotide hybridized to an oligonucleotide component via the oligonucleotide hybridization region. Whether specific hybridization occurs may be determined by factors such as the degree of complementarity between the hybridizing regions of the scaffold polynucleotide, the terminal region of the ssNA fragment, and the oligonucleotide component, as well as the length thereof, salt concentration, GC content, and the temperature at which the hybridization occurs, which may be informed by the melting temperatures (Tm) of the relevant regions.

Complexes may be formed such that an end of an oligonucleotide component is adjacent to an end of a terminal region of an ssNA fragment. Adjacent to refers the terminal nucleotide at the end of the oligonucleotide and the terminal nucleotide end of the terminal region of the ssNA fragment are sufficiently proximal to each other that the terminal nucleotides may be covalently linked, for example, by chemical ligation, enzymatic ligation, or the like. In some embodiments, the ends are adjacent to each other by virtue of the terminal nucleotide at the end of the oligonucleotide and the terminal nucleotide end of the terminal region of the ssNA being hybridized to adjacent nucleotides of the scaffold polynucleotide. The scaffold polynucleotide may be designed to ensure that an end of the oligonucleotide is adjacent to an end of the terminal region of the ssNA fragment.

A scaffold polynucleotide may be designed with one or more uracil bases in place of thymine. In some embodiments, one of the strands in a scaffold adapter duplex may be degraded by generating multiple cut sites at uracil bases, for example by using a uracil-DNA glycosylase and an endonuclease.

Scaffold adapters comprising in-line UMI designs described herein may be configured to connect to one or both ends of an ssNA fragment. In some configurations, scaffold adapters are designed such that the adapter species that connects to the 5′ end of an ssNA comprises an in-line UMI design described herein. In some configurations, scaffold adapters are designed such that the adapter species that connects to the 3′ end of an ssNA comprises an in-line UMI design described herein. In some configurations, scaffold adapters are designed such that the adapter species that connects to the 5′ end of an ssNA comprises an in-line UMI design described herein and the adapter species that connects to the 3′ end of the ssNA does not include an in-line UMI. In some configurations, scaffold adapters are designed such that the adapter species that connects to the 3′ end of an ssNA comprises an in-line UMI design described herein and the adapter species that connects to the 5′ end of the ssNA does not include an in-line UMI. In some configurations, scaffold adapters are designed such that the adapter species that connects to the 5′ end of an ssNA comprises an in-line UMI design described herein and the adapter species that connects to the 3′ end of the ssNA also comprises an in-line UMI design described herein.

Scaffold adapters, oligonucleotide components, and scaffold polynucleotides may be referred to herein as first scaffold adapters (or first scaffold duplexes), first oligonucleotide components (or first oligonucleotides), first unique molecular identifiers (UMIs), and first scaffold polynucleotides; or second scaffold adapters (or second scaffold duplexes), second oligonucleotide components (or second oligonucleotides), second unique molecular identifiers (UMIs), and second scaffold polynucleotides. The terms first and second generally refer to scaffold adapters, or components thereof, that hybridize to and/or are covalently linked to a first end and second end of an ssNA fragment terminus (i.e., a 5′ end and a 3′ end). The terms first end and second end do not always refer to a particular directionality of the ssNA fragment. Accordingly, a first end of an ssNA terminus may be a 5′ end or a 3′ end, and a second end of an ssNA terminus may be a 5′ end or a 3′ end. A first scaffold adapter, or component thereof, may refer to a P5 adapter, or component thereof, or a P7 adapter, or component thereof. A second scaffold adapter, or component thereof, may refer to a P5 adapter, or component thereof, or a P7 adapter, or component thereof.

In some instances, scaffold adapters, oligonucleotide components, and scaffold polynucleotides may be referred to herein as (i) first scaffold adapters (or first scaffold duplexes), first oligonucleotide components (or first oligonucleotides), and first scaffold polynucleotides; (ii) second scaffold adapters (or second scaffold duplexes), second oligonucleotide components (or second oligonucleotides), and second scaffold polynucleotides; (iii) third scaffold adapters (or third scaffold duplexes), third oligonucleotide components (or third oligonucleotides), and third scaffold polynucleotides; or (iv) fourth scaffold adapters (or fourth scaffold duplexes), fourth oligonucleotide components (or fourth oligonucleotides), and fourth scaffold polynucleotides. In such instances (e.g., when scaffold adapters, or components thereof, are combined with a mixture of ssRNA and ssDNA), the terms first and second generally refer to scaffold adapters, or components thereof, that hybridize to and/or are covalently linked to a first end of an ssRNA fragment terminus (i.e., a 5′ end and a 3′ end) and a first end of an ssDNA fragment terminus (i.e., a 5′ end and a 3′ end), respectively. The terms third and fourth generally refer to scaffold adapters, or components thereof, that hybridize to and/or are covalently linked to a second end of an ssRNA fragment terminus (i.e., a 5′ end and a 3′ end) and a second end of an ssDNA fragment terminus (i.e., a 5′ end and a 3′ end), respectively.

Regions that flank a first unique molecular identifier (UMI) may be referred to as a first flank region and a second flank region. A first flank region generally refers to a region in a first oligonucleotide that is proximal to the oligonucleotide end that is adjacent to the ssNA terminus, when a complex is formed (i.e., adjacent to the oligonucleotide-ssNA junction or “ligation terminus”). A second flank region generally refers to a region in a first oligonucleotide that is distal to the oligonucleotide end that is adjacent to the ssNA terminus, when a complex is formed. Regions that flank a second unique molecular identifier (UMI) may be referred to as a third flank region and a fourth flank region. A third flank region generally refers to a region in a second oligonucleotide that is proximal to the oligonucleotide end that is adjacent to the ssNA terminus, when a complex is formed (i.e., adjacent to the oligonucleotide-ssNA junction or “ligation terminus”). A fourth flank region generally refers to a region in a second oligonucleotide that is distal to the oligonucleotide end that is adjacent to the ssNA terminus, when a complex is formed. The terms first flank region, second flank region, third flank region, and fourth flank region do not always refer to a particular directionality of the components within an oligonucleotide. A first flank region and a third flank region may be referred to herein as flank regions or anchor sequences. A second flank region and a fourth flank region may be referred to herein as further flank regions.

In some instances, prior to combining scaffold adapters or components thereof with a nucleic acid sample comprising ssNA, the nucleic acid sample can be treated with a nuclease to remove unwanted nucleic acids. For example, a double-stranded specific nuclease (e.g., T7 nuclease) can be used to digest some or all double-stranded DNA, and scaffolding adapters can then be used to prepare a sequencing library of the remaining nucleic acids as disclosed herein. In an example, a double-stranded specific nuclease is used to digest double-stranded nucleic acids in a sample, leaving intact single-stranded nucleic acids such as those from single-stranded DNA viruses, single-stranded RNA viruses, and single-stranded DNA (e.g., damaged DNA) while digesting double-stranded DNA from a host organism and/or bacteria.

Hybridization and Ligation

Nucleic acid fragments (e.g., ssNA fragments) may be combined with scaffold adapters, or components thereof, thereby generating combined products. Combining ssNA fragments with scaffold adapters, or components thereof, may comprise hybridization and/or ligation (e.g., ligation of hybridization products). A combined product may include an ssNA fragment connected to (e.g., hybridized to and/or ligated to) a scaffold adapter, or component thereof, at one or both ends of the ssNA fragment. A combined product may include an ssNA fragment hybridized to a scaffold adapter, or component thereof, at one or both ends of the ssNA fragment, which may be referred to as a hybridization product. A combined product may include an ssNA fragment ligated to a scaffold adapter, or component thereof, at one or both ends of the ssNA fragment, which may be referred to as a ligation product. In some embodiments, products from a cleavage step (i.e., cleaved products) may be combined with scaffold adapters, or components thereof, thereby generating combined products. Certain methods herein comprise generating sets of combined products (e.g., a first set of combined products and a second set of combined products). In some embodiments, a first set of combined products includes ssNAs connected to (e.g., hybridized to and/or ligated to) scaffold adapters, or components thereof, from a first set of scaffold adapters, or components thereof. In some embodiments, a second set of combined products includes the first set of combined products connected to (e.g., hybridized to and/or ligated to) scaffold adapters, or components thereof, from a second set of scaffold adapters, or components thereof.

ssNAs may be combined with scaffold adapters, or components thereof, under hybridization conditions, thereby generating hybridization products. In some embodiments, the scaffold adapters are provided as pre-hybridized products and the hybridization step includes hybridizing the scaffold adapters to the ssNA. In some embodiments, the scaffold adapter components (i.e., oligonucleotides and scaffold polynucleotides) are provided as individual components and the hybridization step includes hybridizing the scaffold adapter components 1) to each other and 2) to the ssNA. In some embodiments, the scaffold adapter components (i.e., oligonucleotides and scaffold polynucleotides) are provided sequentially as individual components and the hybridization steps includes 1) hybridizing the scaffold polynucleotides to the ssNA, and then 2) hybridizing the oligonucleotides to the oligonucleotide hybridization region of the scaffold polynucleotides. The conditions during the combining step are those conditions in which scaffold adapters, or components thereof (e.g., single-stranded scaffold regions), specifically hybridize to ssNAs having a terminal region or terminal regions that are complementary in sequence with respect to the single-stranded scaffold regions. The conditions during the combining step also may include those conditions in which components of the scaffold adapters (e.g., oligonucleotides and oligonucleotide hybridization regions within the scaffold polynucleotides), specifically hybridize, or remain hybridized, to each other.

Specific hybridization may be affected or influenced by factors such as the degree of complementarity between the single-stranded scaffold regions and the ssNA terminal region(s), or between the oligonucleotides and oligonucleotide hybridization regions, the length thereof, and the temperature at which the hybridization occurs, which may be informed by melting temperatures (Tm) of the single-stranded scaffold regions. Melting temperature generally refers to the temperature at which half of the single-stranded scaffold regions/ssNA terminal regions remain hybridized and half of the single-stranded scaffold regions/ssNA terminal regions dissociate into single strands. The Tm of a duplex may be experimentally determined or predicted using the following formula Tm=81.5+16.6(log₁₀[Na+])+0.41 (fraction G+C)−(60/N), where N is the chain length and [Na+] is less than 1 M. Additional models that depend on various parameters also may be used to predict Tm of relevant regions depending on various hybridization conditions. Approaches for achieving specific nucleic acid hybridization are described, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993).

In some embodiments, a method herein comprises exposing hybridization products to conditions under which an end of an ssNA is joined to an end of a scaffold adapter to which it is hybridized. In particular, a method herein may comprise exposing hybridization products to conditions under which an end of an ssNA is joined to an end of an oligonucleotide component of a scaffold adapter to which it is hybridized. Joining may be achieved by any suitable approach that permits covalent attachment of ssNA to the scaffold adapter and/or oligonucleotide component of a scaffold adapter to which it is hybridized. When one end of an ssNA is joined to an end of a scaffold adapter and/or oligonucleotide component of a scaffold adapter to which it is hybridized, typically one of two attachment events is conducted: 1) the 3′ end of the ssNA to the 5′ end of the oligonucleotide component of the scaffold adapter, or 2) the 5′ end of the ssNA to the 3′ end of the oligonucleotide component of the scaffold adapter. When both ends of an ssNA are each joined to an end of a scaffold adapter and/or oligonucleotide component of a scaffold adapter to which it is hybridized, typically two attachment events are conducted: 1) the 3′ end of the ssNA to the 5′ end of the oligonucleotide component of a first scaffold adapter, and 2) the 5′ end of the ssNA to the 3′ end of the oligonucleotide component of a second scaffold adapter.

In some embodiments, a method herein comprises contacting hybridization products with an agent comprising a ligase activity under conditions in which an end of an ssNA is covalently linked to an end of a scaffold adapter and/or oligonucleotide component of a scaffold adapter to which the target nucleic acid (ssNA) is hybridized. Ligase activity may include, for example, blunt-end ligase activity, nick-sealing ligase activity, sticky end ligase activity, circularization ligase activity, cohesive end ligase activity, DNA ligase activity, RNA ligase activity, single-stranded ligase activity, and double-stranded ligase activity. Ligase activity may include ligating a 5′ phosphorylated end of one polynucleotide to a 3′ OH end of another polynucleotide (5′P to 3′OH). Ligase activity may include ligating a 3′ phosphorylated end of one polynucleotide to a 5′ OH end of another polynucleotide (3′P to 5′OH). Ligase activity may include ligating a 5′ end of an ssNA to a 3′ end of a scaffold adapter and/or oligonucleotide component of a scaffold adapter hybridized thereto in a ligation reaction. Ligase activity may include ligating a 3′ end of an ssNA to a 5′ end of a scaffold adapter and/or oligonucleotide component of a scaffold adapter hybridized thereto in a ligation reaction. Suitable reagents (e.g., ligases) and kits for performing ligation reactions are known and available. For example, Instant Sticky-end Ligase Master Mix available from New England Biolabs (Ipswich, MA) may be used. Ligases that may be used include but are not limited to, for example, T3 ligase, T4 DNA ligase (e.g., at low or high concentration), T7 DNA Ligase, E. coli DNA Ligase, Electro Ligase®, RNA ligases, T4 RNA ligase 1, T4 RNA ligase 2, SplintR® Ligase, RtcB ligase, Taq ligase, and the like and combinations thereof. When needed, a phosphate group may be added at the 5′ end of the oligonucleotide component or ssNA fragment using a suitable kinase, for example, such as T4 polynucleotide kinase (PNK). Such kinases and guidance for using such kinases to phosphorylate 5′ ends are available, for example, from New England BioLabs, Inc. (Ipswich, MA).

In some embodiments, a method comprises covalently linking the adjacent ends of an oligonucleotide component and an ssNA terminal region, thereby generating covalently linked hybridization products. In some embodiments, the covalently linking comprises contacting the hybridization products (e.g., ssNA fragments hybridized to at least one scaffold adapter herein) with an agent comprising a ligase activity under conditions in which the end of an ssNA terminal region is covalently linked to an end of the oligonucleotide component. In some embodiments, a method comprises covalently linking the adjacent ends of a first oligonucleotide component and a first ssNA terminal region, and covalently linking the adjacent ends of a second oligonucleotide component and a second ssNA terminal region, thereby generating covalently linked hybridization products. In some embodiments, the covalently linking comprises contacting hybridization products (e.g., ssNA fragments each hybridized two scaffold adapters herein) with an agent comprising a ligase activity under conditions in which an end of a first ssNA terminal region is covalently linked to an end of a first oligonucleotide component and an end of a second ssNA terminal region is covalently linked to an end of a second oligonucleotide component. In some embodiments, the agent comprising a ligase activity is a T4 DNA ligase. In some embodiments, the T4 DNA ligase is used at an amount between about 1 unit/μl to about 50 units/μl. In some embodiments, the T4 DNA ligase is used at an amount between about 5 unit/μl to about 30 units/μl. In some embodiments, the T4 DNA ligase is used at an amount between about 5 unit/μl to about 15 units/μl. In some embodiments, the T4 DNA ligase is used at about 10 units/μl. In some embodiments, the T4 DNA ligase is used at an amount less than 25 units/μl. In some embodiments, the T4 DNA ligase is used at an amount less than 20 units/μl. In some embodiments, the T4 DNA ligase is used at an amount less than 15 units/μl. In some embodiments, the T4 DNA ligase is used at an amount less than 10 units/μl.

In some embodiments, hybridization products are contacted with a first agent comprising a first ligase activity and a second agent comprising a second ligase activity different than the first ligase activity. For example, the first ligase activity and the second ligase activity independently may be chosen from blunt-end ligase activity, nick-sealing ligase activity, sticky end ligase activity, circularization ligase activity, and cohesive end ligase activity, double-stranded ligase activity, single-stranded ligase activity, 5′P to 3′OH ligase activity, and 3′P to 5′OH ligase activity.

In some embodiments, a method herein comprises joining ssNAs to scaffold adapters and/or oligonucleotide components of scaffold adapters via biocompatible attachments. Methods may include, for example, click chemistry or tagging, which include biocompatible reactions useful for joining biomolecules. In some embodiments, an end of each of the oligonucleotide components comprises a first chemically reactive moiety and an end of each of the ssNAs includes a second chemically reactive moiety. In such embodiments, the first chemically reactive moiety typically is capable of reacting with the second chemically reactive moiety and forming a covalent bond between an oligonucleotide component of a scaffold adapter and an ssNA to which the scaffold adapter is hybridized. In some embodiments, a method herein includes contacting ssNA with one or more chemical agents under conditions in which the second chemically reactive moiety is incorporated at an end of each of the ssNA fragments. In some embodiments, a method herein includes exposing hybridization products to conditions in which the first chemically reactive moiety reacts with the second chemically reactive moiety forming a covalent bond between an oligonucleotide component and an ssNA to which the scaffold adapter is hybridized. In some embodiments, the first chemically reactive moiety is capable of reacting with the second chemically reactive moiety to form a 1,2,3-triazole between the oligonucleotide component and the ssNA to which the scaffold adapter is hybridized. In some embodiments, the first chemically reactive moiety is capable of reacting with the second chemically reactive moiety under conditions comprising copper. The first and second chemically reactive moieties may include any suitable pairings. For example, the first chemically reactive moiety may be chosen from an azide-containing moiety and 5-octadiynyl deoxyuracil, and the second chemically reactive moiety may be independently chosen from an azide-containing moiety, hexynyl and 5-octadiynyl deoxyuracil. In some embodiments, the azide-containing moiety is N-hydroxysuccinimide (NHS) ester-azide.

Covalently linking the adjacent ends of an oligonucleotide and an ssNA fragment produces a covalently linked product, which may be referred to a ligation product. A covalently linked product that includes an ssNA fragment covalently linked to an oligonucleotide component, which remain hybridized to a scaffold polynucleotide, may be referred to as a covalently linked hybridization product. A covalently linked hybridization product may be denatured (e.g., heat-denatured) to separate the ssNA fragment covalently linked to an oligonucleotide component from the scaffold polynucleotide. A covalently linked product that includes an ssNA fragment covalently linked to an oligonucleotide component, which is no longer hybridized to a scaffold polynucleotide (e.g., after denaturing), may be referred to as a single-stranded ligation product. In some instances, portions of a scaffold polynucleotide can be cleaved and/or degraded, for example by using uracil-DNA glycosylase and an endonuclease at one or more uracil bases in the scaffold polynucleotide.

A covalently linked hybridization product and/or single-stranded ligation product may be purified prior to use as input in a downstream application of interest (e.g., amplification; sequencing). For example, covalently linked hybridization products and/or single-stranded ligation products may be purified from certain components present during the combining, hybridization, and/or covalently linking (ligation) steps (e.g., by solid phase reversible immobilization (SPRI), column purification, and/or the like).

In some embodiments, when a method herein include combining an ssNA composition with scaffold adapters herein, or components thereof, and covalently linking the adjacent ends of an oligonucleotide component and an ssNA fragment, the total duration of the combining and covalently linking may be 4 hours or less, 3 hours or less, 2 hours or less, or 1 hour or less. In some embodiments, the total duration of the combining and covalently linking is less than 1 hour.

In some embodiments, a method herein is performed in a single vessel, a single chamber, and/or a single volume (i.e., contiguous volume), including but not limited to on a microfluidic device. In some embodiments, combining an ssNA composition with scaffold adapters herein, or components thereof, and covalently linking the adjacent ends of an oligonucleotide component and an ssNA fragment are performed in a single vessel, a single chamber, and/or a single volume (i.e., contiguous volume), including but not limited to on a microfluidic device. In some embodiments, a method herein is performed in a collection of wells, droplets, emulsion, partitions, or other reaction volumes, including but not limited to on a microfluidic device. In some embodiments, combining an ssNA composition with scaffold adapters herein, or components thereof, and covalently linking the adjacent ends of an oligonucleotide component and an ssNA fragment are performed in a collection of wells, droplets, emulsion, partitions, or other reaction volumes, including but not limited to on a microfluidic device. In some instances, the collection of reaction volumes are prepared such that a majority or all of the reaction volumes comprise at most one ssNA. In some instances, the collection of reaction volumes are prepared such that a majority or all of the reaction volumes comprise at most 2, 3, 4, 5, 6, 7, 8, 9, 10, 20, 30, 40, 50, 60, 70, 80, 90, 100, 1000, 2000, 3000, 4000, 5000, 6000, 7000, 8000, 9000, 10000, 20000, 30000, 40000, 50000, 60000, 70000, 80000, 90000, 100000, or more ssNA. Partitioning one or a limited number of ssNA into reaction volumes can provide favorable reaction kinetics, such as increasing the library conversion of rare species of sample nucleic acids.

Modified Nucleotides

In some embodiments, a scaffold adapter, or component thereof, comprises one or more modified nucleotides. In some embodiments, a UMI and/or a flank region adjacent to a UMI comprises one or more modified nucleotides. Modified nucleotides may be referred to as modified bases or non-canonical bases and may include, for example, nucleotides conjugated to a member of a binding pair, blocked nucleotides, non-natural nucleotides, nucleotide analogues, peptide nucleic acid (PNA) nucleotides, Morpholino nucleotides, locked nucleic acid (LNA) nucleotides, bridged nucleic acid (BNA) nucleotides, glycol nucleic acid (GNA) nucleotides, threose nucleic acid (TNA) nucleotides, and the like and combinations thereof. In certain configurations, a scaffold adapter, or component thereof (e.g., a UMI and/or a flank region adjacent to a UMI) comprises one or more nucleotides with modifications chosen from one or more of amino modifier, biotinylation, thiol, alkynes, 2′-O-methoxy-ethyl Bases (2′-MOE), RNA, fluoro bases, iso (iso-dG, iso-DC), inverted, methyl, nitro, phos, and the like.

In some embodiments, a scaffold adapter, or component thereof (e.g., a UMI and/or a flank region adjacent to a UMI), comprises one or more modified nucleotides within a duplex region, within a scaffold region, at one end, or at both ends of the scaffold adapter, or component thereof. In some embodiments, a scaffold adapter, or component thereof, comprises one or more unpaired modified nucleotides. In some embodiments, a scaffold adapter, or component thereof, comprises one or more unpaired modified nucleotides at one end of the adapter. In some embodiments, a scaffold adapter, or component thereof, comprises one or more unpaired modified nucleotides at the end of the adapter opposite to the end that hybridizes to a target nucleic acid (e.g., an end comprising a single-stranded scaffold region). A modified nucleotide may be present at the end of the strand having a 3′ terminus or at the end of the strand having a 5′ terminus.

In some embodiments, an oligonucleotide component comprises one or more modified nucleotides. In some embodiments, the one or more modified nucleotides are capable of blocking covalent linkage of the oligonucleotide component to another oligonucleotide, polynucleotide, or nucleic acid molecule. In some embodiments, an oligonucleotide component comprises one or more modified nucleotides at an end not adjacent to the ssNA. In some embodiments, a scaffold polynucleotide comprises one or more modified nucleotides. In some embodiments, the one or more modified nucleotides are capable of blocking covalent linkage of the scaffold polynucleotide to another oligonucleotide, polynucleotide, or nucleic acid molecule. A scaffold polynucleotide may comprise the one or more modified nucleotides at one or both ends of the polynucleotide. In some embodiments, the one or more modified nucleotides comprise a ligation-blocking modification.

In some embodiments, a scaffold adapter, or component thereof, comprises one or more blocked nucleotides. In one example, a scaffold adapter, or component thereof, may comprise one or more modified nucleotides that are capable of blocking hybridization to a nucleotide in another scaffold adapter, or component thereof. In some instances, the one or more modified nucleotides are capable of blocking ligation to a nucleotide in another scaffold adapter, or component thereof. In another example, a scaffold adapter, or component thereof, may comprise one or more modified nucleotides that are capable of blocking hybridization to a nucleotide in a target nucleic acid (e.g., ssNA). In some instances, the one or more modified nucleotides are capable of blocking ligation to a nucleotide in a target nucleic acid. In some embodiments, one or both ends of a scaffold polynucleotide include a blocking modification and/or the end of an oligonucleotide component not adjacent to an ssNA fragment may include a blocking modification. A blocking modification refers to a modified end that cannot be linked to the end of another nucleic acid component using an approach employed to covalently link the adjacent ends of an oligonucleotide component and an ssNA fragment. In certain embodiments, the blocking modification is a ligation-blocking modification. Examples of blocking modifications which may be included at one or both ends of a scaffold polynucleotide and/or the end of an oligonucleotide component not adjacent to the ssNA, include the absence of a 3′ OH, and an inaccessible 3′ OH. Non-limiting examples of blocking modifications in which an end has an inaccessible 3′ OH include: an amino modifier, an amino linker, a spacer, an isodeoxy-base, a dideoxy base, an inverted dideoxy base, a 3′ phosphate, and the like. In some embodiments, a scaffold adapter, or component thereof, comprises one or more modified nucleotides that are incapable of binding to a natural nucleotide.

In some embodiments, one or more modified nucleotides comprise an isodeoxy-base. In some embodiments, one or more modified nucleotides comprise isodeoxy-guanine (iso-dG). In some embodiments, one or more modified nucleotides comprise isodeoxy-cytosine (iso-dC). Iso-dC and iso-dG are chemical variants of cytosine and guanine, respectively. Iso-dC can hydrogen bond with iso-dG but not with unmodified guanine (natural guanine). Iso-dG can base pair with Iso-dC but not with unmodified cytosine (natural cytosine). A scaffold adapter, or component thereof, containing iso-dC can be designed so that it hybridizes to a complementary oligo containing iso-dG but cannot hybridize to any naturally occurring nucleic acid sequence.

In some embodiments, one or more modified nucleotides comprise epigenetic-associated modifications, including but not limited to methylation, hydroxymethylation, and carboxylation. Example epigenetic-associated modifications include carboxycytosine, 5-methylcytosine (5mC) and its oxidative derivatives (e.g., 5-hydroxymethylcytosine (5hmC), 5-formylcytosine (5fC), and 5-arboxylcytosine (5caC)), N(6)-methyladenine (6 mA), N4-methylcytosine (4mC), N(6)-methyladenosine (m(6)A), pseudouridine (ψ), 5-methylcytidine (m(5)C), hydroxymethyl uracil, 2′-O-methylation at the 3′ end, tRNA modifications, miRNA modifications, and snRNA modifications.

In some embodiments, one or more modified nucleotides comprise a dideoxy-base. In some embodiments, one or more modified nucleotides comprise dideoxy-cytosine. In some embodiments, one or more modified nucleotides comprise an inverted dideoxy-base. In some embodiments, one or more modified nucleotides comprise inverted dideoxy-thymine. For example, an inverted dideoxy-thymine located at the 5′ end of a sequence can prevent unwanted 5′ ligations.

In some embodiments, one or more modified nucleotides comprise a spacer. In some embodiments, one or more modified nucleotides comprise a C3 spacer. A C3 spacer phosphoramidite can be incorporated internally or at the 5′-end of an oligonucleotide. Multiple C3 spacers can be added at either end of a scaffold adapter, or component thereof, to introduce a long hydrophilic spacer arm (e.g., for the attachment of fluorophores or other pendent groups). Other spacers include, for example, photo-cleavable (PC) spacers, hexanediol, spacer 9, spacer 18, 1′,2′-dideoxyribose (dSpacer), and the like.

In some embodiments, a modified nucleotide comprises an amino linker or amino blocker. In some embodiments, a modified nucleotide comprises an amino linker C6 (e.g., a 5′ amino linker C6 or a 3′ amino linker C6). In one example, an amino linker C6 can be used to incorporate an active primary amino group onto the 5′-end of an oligonucleotide. This can then be conjugated to a ligand. The amino group then becomes internal to the 5 end ligand. The amino group is separated from the 5′-end nucleotide base by a 6-carbon spacer arm to reduce steric interaction between the amino group and the oligo. In some embodiments, a modified nucleotide comprises an amino linker C12 (e.g., a 5′ amino linker C12 or a 3′ amino linker C12). In one example, an amino linker C12 can be used to incorporate an active primary amino group onto the 5′-end of an oligonucleotide. The amino group is separated from the 5′-end nucleotide base by a 12-carbon spacer arm to minimize steric interaction between the amino group and the oligo.

In some embodiments, a modified nucleotide comprises a member of a binding pair. Binding pairs may include, for example, antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, chemical reactive group/complementary chemical reactive group, digoxigenin moiety/anti-digoxigenin antibody, fluorescein moiety/anti-fluorescein antibody, steroid/steroid-binding protein, operator/repressor, nuclease/nucleotide, lectin/polysaccharide, active compound/active compound receptor, hormone/hormone receptor, enzyme/substrate, oligonucleotide or polynucleotide/its corresponding complement, the like or combinations thereof. In some embodiments, a modified nucleotide comprises biotin.

In some embodiments, a modified nucleotide comprises a first member of a binding pair (e.g., biotin); and a second member of a binding pair (e.g., streptavidin) is conjugated to a solid support or substrate. A solid support or substrate can be any physically separable solid to which a member of a binding pair can be directly or indirectly attached including, but not limited to, surfaces provided by microarrays and wells, and particles such as beads (e.g., paramagnetic beads, magnetic beads, microbeads, nanobeads), microparticles, and nanoparticles. Solid supports also can include, for example, chips, columns, optical fibers, wipes, filters (e.g., flat surface filters), one or more capillaries, glass and modified or functionalized glass (e.g., controlled-pore glass (CPG)), quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polybutylene, polyurethanes, TEFLON™, polyethylene, polypropylene, polyamide, polyester, polyvinylidenedifluoride (PVDF), and the like), polysaccharides, nylon or nitrocellulose, resins, silica or silica-based materials including silicon, silica gel, and modified silicon, Sephadex®, Sepharose®, carbon, metals (e.g., steel, gold, silver, aluminum, silicon and copper), inorganic glasses, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In some embodiments, a solid support or substrate may be coated using passive or chemically-derivatized coatings with any number of materials, including polymers, such as dextrans, acrylamides, gelatins or agarose. Beads and/or particles may be free or in connection with one another (e.g., sintered). In some embodiments, a solid support can be a collection of particles. In some embodiments, the particles can comprise silica, and the silica may comprise silica dioxide. In some embodiments, the silica can be porous, and in certain embodiments the silica can be non-porous. In some embodiments, the particles further comprise an agent that confers a paramagnetic property to the particles. In certain embodiments, the agent comprises a metal, and in certain embodiments the agent is a metal oxide, (e.g., iron or iron oxides, where the iron oxide contains a mixture of Fe2+ and Fe3+). A member of a binding pair may be linked to a solid support by covalent bonds or by non-covalent interactions and may be linked to a solid support directly or indirectly (e.g., via an intermediary agent such as a spacer molecule or biotin).

In some embodiments, a scaffold polynucleotide, an oligonucleotide component (e.g., a UMI and/or a flank region adjacent to a UMI), or both, include one or more non-natural nucleotides, also referred to as nucleotide analogs. Non-limiting examples of non-natural nucleotides that may be included in a scaffold polynucleotide, an oligonucleotide component, or both include LNA (locked nucleic acid), PNA (peptide nucleic acid), FANA (2′-deoxy-2′-fluoroarabinonucleotide), GNA (glycol nucleic acid), TNA (threose nucleic acid), 2′-O-Me RNA, 2′-fluoro RNA, Morpholino nucleotides, and any combination thereof.

End Treatments

In some embodiments, a method herein comprises contacting a nucleic acid composition comprising single-stranded nucleic acid (ssNA) with an agent comprising an end treatment activity under conditions in which single-stranded nucleic acid (ssNA) molecules are end treated, thereby generating an end treated ssNA composition. End treatments can include but are not limited to phosphorylation, dephosphorylation, methylation, demethylation, oxidation, de-oxidation, base modification, extension, polymerization, and combinations thereof. End treatments can be conducted with enzymes, including but not limited to ligases, polynucleotide kinases (PNK), terminal transferases, methyltransferases, methylases (e.g., 3′ methylases, 5′ methylases), polymerases (e.g., poly A polymerases), oxidases, and combinations thereof.

In some embodiments, a method herein comprises contacting a nucleic acid composition comprising single-stranded nucleic acid (ssNA) with an agent comprising a phosphatase activity under conditions in which single-stranded nucleic acid (ssNA) molecules are dephosphorylated, thereby generating a dephosphorylated ssNA composition. In some embodiments, a method herein comprises contacting a scaffold adapter, or component thereof, with an agent comprising a phosphatase activity under conditions in which the scaffold adapter, or component thereof, is dephosphorylated, thereby generating a dephosphorylated scaffold adapter, or component thereof (e.g., a dephosphorylated oligonucleotide; a dephosphorylated scaffold polynucleotide). Generally, an ssNA composition and/or scaffold adapters, or components thereof, are dephosphorylated prior to a combining step (i.e., prior to hybridization). ssNAs may be dephosphorylated and then subsequently phosphorylated prior to a combining step (i.e., prior to hybridization). Scaffold adapters, or components thereof, may be dephosphorylated and then subsequently phosphorylated prior to a combining step (i.e., prior to hybridization). Scaffold adapters, or components thereof, may be dephosphorylated and then not phosphorylated prior to a combining step (i.e., prior to hybridization). Scaffold adapters, or components thereof, may be dephosphorylated, not phosphorylated prior to a combining step (i.e., prior to hybridization), and then phosphorylated after a combining step (i.e., after hybridization) and prior to or during a ligation step. Reagents and kits for carrying out dephosphorylation of nucleic acids are known and available. For example, target nucleic acids (e.g., ssNAs) and/or scaffold adapters, or components thereof, can be treated with a phosphatase (i.e., an enzyme that uses water to cleave a phosphoric acid monoester into a phosphate ion and an alcohol).

In some embodiments, a method herein comprises contacting a nucleic acid composition comprising single-stranded nucleic acid (ssNA) with an agent comprising a phosphoryl transfer activity under conditions in which a 5′ phosphate is added to a 5 end of ssNAs. In some embodiments, a method herein comprises contacting a dephosphorylated ssNA composition with an agent comprising a phosphoryl transfer activity under conditions in which a 5′ phosphate is added to a 5 end of an ssNA. In some embodiments, a method herein comprises contacting a scaffold adapter, or component thereof, with an agent comprising a phosphoryl transfer activity under conditions in which a 5′ phosphate is added to a 5′ end of a scaffold adapter, or component thereof. In some embodiments, a method herein comprises contacting a dephosphorylated scaffold adapter, or component thereof, with an agent comprising a phosphoryl transfer activity under conditions in which a 5′ phosphate is added to a 5′ end of a scaffold adapter, or component thereof. In certain instances, an ssNA composition and/or scaffold adapters, or components thereof, are phosphorylated prior to a combining step (i.e., prior to hybridization). 5′ phosphorylation of nucleic acids can be conducted by a variety of techniques. For example an ssNA composition and/or scaffold adapters, or components thereof, can be treated with a polynucleotide kinase (PNK) (e.g., T4 PNK), which catalyzes the transfer and exchange of Pi from the y position of ATP to the 5′-hydroxyl terminus of polynucleotides (double- and single-stranded DNA and RNA) and nucleoside 3′-monophosphates. Suitable reaction conditions include, e.g., incubation of the nucleic acids with PNK in 1×PNK reaction buffer (e.g., 70 mM Tris-HCl, 10 mM MgCl₂, 5 mM DTT, pH 7.6 @ 25° C.) for 30 minutes at 37° C.; and incubation of the nucleic acids with PNK in T4 DNA ligase buffer (e.g., 50 mM Tris-HCl, 10 mM MgCl₂, 1 mM ATP, 10 mM DTT, pH 7.5 @ 25° C.) for 30 minutes at 37° C. Optionally, following the phosphorylation reaction, the PNK may be heat inactivated, e.g., at 65° C. for 20 minutes.

In some embodiments, a method herein does not include use of an agent comprising a phosphoryl transfer activity. In some embodiments, methods do not include producing the 5′ phosphorylated ssNAs by phosphorylating the 5′ ends of ssNAs from a nucleic acid sample. In certain instances, a nucleic acid sample comprises ssNAs with natively phosphorylated 5′ ends. In some embodiments, methods do not include producing the 5′ phosphorylated scaffold adapters, or components thereof, by phosphorylating the 5′ ends of scaffold adapters, or components thereof.

Cleavage

In some embodiments, ssNAs, scaffold adapters, and/or hybridization products (e.g., scaffold adapters hybridized to ssNAs) are cleaved or sheared prior to, during, or after a method described herein. In some embodiments, ssNAs, scaffold adapters, and/or hybridization products are cleaved or sheared at a cleavage site. In some embodiments, scaffold adapters and/or hybridization products are cleaved or sheared at a cleavage site within a hairpin loop. In some embodiments, scaffold adapters and/or hybridization products are cleaved or sheared at a cleavage site at an internal location in a scaffold adapter (e.g., within a duplex region of a scaffold adapter). In some embodiments, scaffold adapters are cleaved at a cleavage site (e.g., a uracil) at an internal location present only on the scaffold polynucleotide but not the complementary oligonucleotide component. Thus, in some embodiments, a scaffold polynucleotide comprises one or more uracil bases, and an oligonucleotide component comprises no uracil bases. In some embodiments, circular hybridization products are cleaved or sheared prior to, during, or after a method described herein. In some embodiments, nucleic acids, such as, for example, cellular nucleic acids and/or large fragments (e.g., greater than 500 base pairs in length) are cleaved or sheared prior to, during, or after a method described herein. Large fragments may be referred to as high molecular weight (HMW) nucleic acid, HMW DNA or HMW RNA. HMW nucleic acid fragments may include fragments greater than about 500 bp, about 600 bp, about 700 bp, about 800 bp, about 900 bp, about 1000 bp, about 2000 bp, about 3000 bp, about 4000 bp, about 5000 bp, about 10,000 bp, or more. The term “shearing” or “cleavage” generally refers to a procedure or conditions in which a nucleic acid molecule may be severed into two (or more) smaller nucleic acid molecules. Such shearing or cleavage can be sequence specific, base specific, or nonspecific, and can be accomplished by any of a variety of methods, reagents or conditions, including, for example, chemical, enzymatic, and physical (e.g., physical fragmentation). Sheared or cleaved nucleic acids may have a nominal, average or mean length of about 5 to about 10,000 base pairs, about 100 to about 1,000 base pairs, about 100 to about 500 base pairs, or about 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000 or 9000 base pairs.

Sheared or cleaved nucleic acids can be generated by a suitable method, non-limiting examples of which include physical methods (e.g., shearing, e.g., sonication, ultrasonication, French press, heat, UV irradiation, the like), enzymatic processes (e.g., enzymatic cleavage agents (e.g., a suitable nuclease, a suitable restriction enzyme), chemical methods (e.g., alkylation, DMS, piperidine, acid hydrolysis, base hydrolysis, heat, the like, or combinations thereof), ultraviolet (UV) light (e.g., at a photo-cleavable site (e.g., comprising a photo-cleavable spacer), the like or combinations thereof. The average, mean or nominal length of the resulting nucleic acid fragments can be controlled by selecting an appropriate fragment-generating method.

The term “cleavage agent” generally refers to an agent, sometimes a chemical or an enzyme that can cleave a nucleic acid at one or more specific or non-specific sites. Specific cleavage agents often cleave specifically according to a particular nucleotide sequence at a particular site, which may be referred to as a cleavage site. Cleavage agents may include enzymatic cleavage agents, chemical cleavage agents, and light (e.g., ultraviolet (UV) light).

Examples of enzymatic cleavage agents include without limitation endonucleases; deoxyribonucleases (DNase; e.g., DNase I, II); ribonucleases (RNase; e.g., RNAse A, RNAse E, RNAse F, RNAse H, RNAse III, RNAse L, RNAse P, RNAse PhyM, RNAse T1, RNAse T2, RNAse U2, and RNAse V); endonuclease VIII; CLEAVASE enzyme; TAQ DNA polymerase; E. coli DNA polymerase I; eukaryotic structure-specific endonucleases; murine FEN-1 endonucleases; nicking enzymes; type I, II or III restriction endonucleases (i.e., restriction enzymes) such as Acc I, AciI, Afl III, Alu I, Alw44 I, Apa I, Asn I, Ava I, Ava II, BamH I, Ban II, Bcl I, Bgl I, Bgl II, Bln I, Bsm I, BssH II, BstE II, BstUI, Cfo I, Cla I, Dde I, Dpn I, Dra I, EcIX I, EcoR I, EcoR I, EcoR II, EcoR V, Hae II, Hae II, HhaI, Hind II, Hind III, Hpa I, Hpa II, Kpn I, Ksp I, Maell, McrBC, Mlu I, MIuN I, Msp I, Nci I, Nco I, Nde I, Nde II, Nhe I, Not I, Nru I, Nsi I, Pst I, Pvu I, Pvu II, Rsa I, Sac I, Sal I, Sau3A I, Sca I, ScrF I, Sfi I, Sma I, Spe I, Sph I, Ssp I, Stu I, Sty I, Swa I, Taq I, Xba I, Xho I; glycosylases (e.g., uracil-DNA glycolsylase (UDG), 3-methyladenine DNA glycosylase, 3-methyladenine DNA glycosylase II, pyrimidine hydrate-DNA glycosylase, FaPy-DNA glycosylase, thymine mismatch-DNA glycosylase (e.g., hypoxanthine-DNA glycosylase, uracil DNA glycosylase (UDG), 5-Hydroxymethyluracil DNA glycosylase (HmUDG), 5-Hydroxymethylcytosine DNA glycosylase, or 1,N6-etheno-adenine DNA glycosylase); exonucleases (e.g., exonuclease I, exonuclease II, exonuclease III, exonuclease IV, exonuclease V, exonuclease VI, exonuclease VII, exonuclease VIII); 5′ to 3′ exonucleases (e.g. exonuclease 1l); 3′ to 5′ exonucleases (e.g. exonuclease 1); poly(A)-specific 3′ to 5′ exonucleases; ribozymes; DNAzymes; and the like and combinations thereof.

In some embodiments, a cleavage site comprises a restriction enzyme recognition site. In some embodiments, a cleavage agent comprises a restriction enzyme. In some embodiments, a cleavage site comprises a rare-cutter restriction enzyme recognition site (e.g., a NotI recognition sequence). In some embodiments, a cleavage agent comprises a rare-cutter enzyme (e.g., a rare-cutter restriction enzyme). A rare-cutter enzyme generally refers to a restriction enzyme with a recognition sequence which occurs only rarely in a genome (e.g., a human genome). An example is NotI, which cuts after the first GC of a 5′-GCGGCCGC-3′ sequence. Restriction enzymes with seven and eight base pair recognition sequences often are considered as rare-cutter enzymes.

Cleavage methods and procedures for selecting restriction enzymes for cutting DNA at specific sites are well known to the skilled artisan. For example, many suppliers of restriction enzymes provide information on conditions and types of DNA sequences cut by specific restriction enzymes, including New England BioLabs, Pro-Mega Biochems, Boehringer-Mannheim, and the like. Enzymes often are used under conditions that will enable cleavage of the DNA with about 95%-100% efficiency, preferably with about 98%-100% efficiency.

In some embodiments, a cleavage site comprises one or more ribonucleic acid (RNA) nucleotides. In some embodiments, a cleavage site comprises a single stranded portion comprising one or more RNA nucleotides. In some embodiments, the singe stranded portion is flanked by duplex portions. In some embodiments, the singe stranded portion is a hairpin loop. In some embodiments, a cleavage site comprises one RNA nucleotide. In some embodiments, a cleavage site comprises two RNA nucleotides. In some embodiments, a cleavage site comprises three RNA nucleotides. In some embodiments, a cleavage site comprises four RNA nucleotides. In some embodiments, a cleavage site comprises five RNA nucleotides. In some embodiments, a cleavage site comprises more than five RNA nucleotides. In some embodiments, a cleavage site comprises one or more RNA nucleotides chosen from adenine (A), cytosine (C), guanine (G), and uracil (U). In some embodiments, a cleavage site comprises one or more RNA nucleotides chosen from adenine (A), cytosine (C), and guanine (G). In some embodiments, a cleavage site comprises no uracil (U). In some embodiments, a cleavage site comprises one or more RNA nucleotides comprising guanine (G). In some embodiments, a cleavage site comprises one or more RNA nucleotides consisting of guanine (G). In some embodiments, a cleavage site comprises one or more RNA nucleotides comprising cytosine (C). In some embodiments, a cleavage site comprises one or more RNA nucleotides consisting of cytosine (C). In some embodiments, a cleavage site comprises one or more RNA nucleotides comprising adenine (A). In some embodiments, a cleavage site comprises one or more RNA nucleotides consisting of adenine (A). In some embodiments, a cleavage site comprises one or more RNA nucleotides consisting of adenine (A), cytosine (C), and guanine (G). In some embodiments, a cleavage site comprises one or more RNA nucleotides consisting of adenine (A) and cytosine (C). In some embodiments, a cleavage site comprises one or more RNA nucleotides consisting of adenine (A) and guanine (G). In some embodiments, a cleavage site comprises one or more RNA nucleotides consisting of cytosine (C) and guanine (G). In some embodiments, a cleavage agent comprises a ribonuclease (RNAse). In some embodiments, an RNAse is an endoribonuclease. An RNAse may be chosen from one or more of RNAse A, RNAse E, RNAse F, RNAse H, RNAse III, RNAse L, RNAse P, RNAse PhyM, RNAse T1, RNAse T2, RNAse U2, and RNAse V.

In some embodiments, a cleavage site comprises a photo-cleavable spacer or photo-cleavable modification. Photo-cleavable modifications may contain, for example, a photolabile functional group that is cleavable by ultraviolet (UV) light of specific wavelength (e.g., 300-350 nm). An example photo-cleavable spacer (available from Integrated DNA Technologies; product no. 1707) is a 10-atom linker arm that can only be cleaved when exposed to UV light within the appropriate spectral range. An oligonucleotide comprising a photo-cleavable spacer can have a 5′ phosphate group that is available for subsequent ligase reactions. Photo-cleavable spacers can be placed between DNA bases or between an oligo and a terminal modification (e.g., a fluorophore). In such embodiments, ultraviolet (UV) light may be considered as a cleavage agent.

In some embodiments, a cleavage site comprises a diol. For example, a cleavage site may comprise vicinal diol incorporated in a 5′ to 5′ linkage. Cleavage sites comprising a diol may be chemically cleaved, for example, using a periodate. In some embodiments, a cleavage site comprises a blunt end restriction enzyme recognition site. Cleavage sites comprising a blunt end restriction enzyme recognition site may be cleaved by a blunt end restriction enzyme.

Nick Seal and Fill-In

In some embodiments, a method herein comprises performing a nick seal reaction (e.g., using a DNA ligase or other suitable enzyme, and, in certain instances, a kinase adapted to 5′ phosphorylate nucleic acids (e.g., a polynucleotide kinase (PNK)). In some embodiments, a method herein comprises performing a fill-in reaction. For example, when scaffold adapters are present as duplexes, some or all of the duplexes may include an overhang at the end of the duplex opposite the end that hybridizes to the ssNAs. When such duplex overhangs exist, subsequent to the combining, a method herein may further include filling in the overhangs formed by the duplexes. In some embodiments, a fill-in reaction is performed to generate a blunt-ended hybridization product. Any suitable reagent for carrying out a fill-in reaction may be used. Polymerases suitable for performing fill-in reactions include, e.g., DNA polymerase I, large (Klenow) fragment of DNA polymerase I, T4 DNA polymerase, Bacillus stearothermophilus (Bst) DNA polymerase, thermostable DNA polymerases (e.g., from hyperthermophilic marine Archaea), 9° N™ DNA Polymerase (GENBANK accession no. AAA88769.1), THERMINATOR polymerase (9°N™ DNA Polymerase with mutations: D141A, B143A, A485L), and the like. In some embodiments, a strand displacing polymerase is used (e.g., Bst DNA polymerase).

Exonuclease Treatment

In some embodiments, nucleic acid (e.g., RNA-DNA duplexes, hybridization products; circularized hybridization products) is treated with an exonuclease. In some embodiments, RNA in an RNA-DNA duplex (e.g., an RNA-DNA duplex generated by first strand cDNA synthesis) is treated with an exonuclease. Exonucleases are enzymes that work by cleaving nucleotides one at a time from the end of a polynucleotide chain through a hydrolyzing reaction that breaks phosphodiester bonds at either the 3′ or the 5′ end. Exonucleases include, for example, DNAses, RNAses (e.g., RNAseH), 5′ to 3′ exonucleases (e.g. exonuclease II), 3′ to 5′ exonucleases (e.g. exonuclease I), and poly(A)-specific 3′ to 5′ exonucleases. In some embodiments, exonuclease activity is provided by a reverse transcriptase (e.g., RNAse activity provided by M-MLV reverse transcriptase having a fully functional RNAseH domain). In some embodiments, hybridization products are treated with an exonuclease to remove contaminating nucleic acids such as, for example, single stranded oligonucleotides, nucleic acid fragments, or RNA from an RNA-DNA duplex. In some embodiments, circularized hybridization products are treated with an exonuclease to remove any non-circularized hybridization products, non-hybridized oligonucleotides, non-hybridized target nucleic acids, oligonucleotide dimers, and the like and combinations thereof.

Samples

Provided herein are methods and compositions for processing and/or analyzing nucleic acid. Nucleic acid or a nucleic acid mixture utilized in methods and compositions described herein may be isolated from a sample obtained from a subject (e.g., a test subject). A subject can be any living or non-living organism, including but not limited to a human, a non-human animal, a plant, a bacterium, a fungus, a protist or a pathogen. Any human or non-human animal can be selected, and may include, for example, mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female (e.g., woman, a pregnant woman). A subject may be any age (e.g., an embryo, a fetus, an infant, a child, an adult). A subject may be a cancer patient, a patient suspected of having cancer, a patient in remission, a patient with a family history of cancer, and/or a subject obtaining a cancer screen. A subject may be a patient having an infection or infectious disease or infected with a pathogen (e.g., bacteria, virus, fungus, protozoa, and the like), a patient suspected of having an infection or infectious disease or being infected with a pathogen, a patient recovering from an infection, infectious disease, or pathogenic infection, a patient with a history of infections, infectious disease, pathogenic infections, and/or a subject obtaining an infectious disease or pathogen screen. A subject may be a transplant recipient. A subject may be a patient undergoing a microbiome analysis. In some embodiments, a test subject is a female. In some embodiments, a test subject is a human female. In some embodiments, a test subject is a male. In some embodiments, a test subject is a human male.

A nucleic acid sample may be isolated or obtained from any type of suitable biological specimen or sample (e.g., a test sample). A nucleic acid sample may be isolated or obtained from a single cell, a plurality of cells (e.g., cultured cells), cell culture media, conditioned media, a tissue, an organ, or an organism (e.g., bacteria, yeast, or the like). In some embodiments, a nucleic acid sample is isolated or obtained from a cell(s), tissue, organ, and/or the like of an animal (e.g., an animal subject). In some embodiments, a nucleic acid sample is isolated or obtained from a source such as bacteria, yeast, insects (e.g., Drosophila), mammals, amphibians (e.g., frogs (e.g., Xenopus)), viruses, plants, or any other mammalian or non-mammalian nucleic acid sample source.

A nucleic acid sample may be isolated or obtained from an extant organism or animal. In some instances, a nucleic acid sample may be isolated or obtained from an extinct (or “ancient”) organism or animal (e.g., an extinct mammal; an extinct mammal from the genus Homo). In some instances, a nucleic acid sample may be obtained as part of a diagnostic analysis.

In some instances, a nucleic acid sample may be obtained as part of a forensics analysis. In some embodiments, a single-stranded nucleic acid library preparation (ssPrep) method described herein is applied to a forensic sample or specimen. A forensic sample or specimen may include any biological substance that contains nucleic acid. For example, a forensic sample or specimen may include blood, semen, hair, skin, sweat, saliva, decomposed tissue, bone, fingernail scrapings, licked stamps/envelopes, sluff, touch DNA, razor residue, and the like.

A sample or test sample may be any specimen that is isolated or obtained from a subject or part thereof (e.g., a human subject, a pregnant female, a cancer patient, a patient having an infection or infectious disease, a transplant recipient, a fetus, a tumor, an infected organ or tissue, a transplanted organ or tissue, a microbiome). A sample sometimes is from a pregnant female subject bearing a fetus at any stage of gestation (e.g., first, second or third trimester for a human subject), and sometimes is from a post-natal subject. A sample sometimes is from a pregnant subject bearing a fetus that is euploid for all chromosomes, and sometimes is from a pregnant subject bearing a fetus having a chromosome aneuploidy (e.g., one, three (i.e., trisomy (e.g., T21, T18, T13)), or four copies of a chromosome) or other genetic variation. Non-limiting examples of specimens include fluid or tissue from a subject, including, without limitation, blood or a blood product (e.g., serum, plasma, or the like), umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample (e.g., from pre-implantation embryo; cancer biopsy), celocentesis sample, cells (blood cells, placental cells, embryo or fetal cells, fetal nucleated cells or fetal cellular remnants, normal cells, abnormal cells (e.g., cancer cells)) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a biological sample is a cervical swab from a subject. A fluid or tissue sample from which nucleic acid is extracted may be acellular (e.g., cell-free). In some embodiments, a fluid or tissue sample may contain cellular elements or cellular remnants. In some embodiments, fetal cells or cancer cells may be included in the sample.

A sample can be a liquid sample. A liquid sample can comprise extracellular nucleic acid (e.g., circulating cell-free DNA). Examples of liquid samples include, but are not limited to, blood or a blood product (e.g., serum, plasma, or the like), urine, cerebrospinal fluid, saliva, sputum, biopsy sample (e.g., liquid biopsy for the detection of cancer), a liquid sample described above, the like or combinations thereof. In certain embodiments, a sample is a liquid biopsy, which generally refers to an assessment of a liquid sample from a subject for the presence, absence, progression or remission of a disease (e.g., cancer). A liquid biopsy can be used in conjunction with, or as an alternative to, a sold biopsy (e.g., tumor biopsy). In certain instances, extracellular nucleic acid is analyzed in a liquid biopsy.

In some embodiments, a biological sample may be blood, plasma or serum. The term “blood” encompasses whole blood, blood product or any fraction of blood, such as serum, plasma, buffy coat, or the like as conventionally defined. Blood or fractions thereof often comprise nucleosomes. Nucleosomes comprise nucleic acids and are sometimes cell-free or intracellular. Blood also comprises buffy coats. Buffy coats are sometimes isolated by utilizing a ficoll gradient. Buffy coats can comprise white blood cells (e.g., leukocytes, T-cells, B-cells, platelets, and the like). Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3 to 40 milliliters, between 5 to 50 milliliters) often is collected and can be stored according to standard procedures prior to or after preparation.

An analysis of nucleic acid found in a subject's blood may be performed using, e.g., whole blood, serum, or plasma. An analysis of fetal DNA found in maternal blood, for example, may be performed using, e.g., whole blood, serum, or plasma. An analysis of tumor or cancer DNA found in a patient's blood, for example, may be performed using, e.g., whole blood, serum, or plasma. An analysis of pathogen DNA found in a patient's blood, for example, may be performed using, e.g., whole blood, serum, or plasma. An analysis of transplant DNA found in a transplant recipient's blood, for example, may be performed using, e.g., whole blood, serum, or plasma. Methods for preparing serum or plasma from blood obtained from a subject (e.g., a maternal subject; patient; cancer patient) are known. For example, a subject's blood (e.g., a pregnant woman's blood; patient's blood; cancer patient's blood) can be placed in a tube containing EDTA or a specialized commercial product such as Cell-Free DNA BCT (Streck, Omaha, NE) or Vacutainer SST (Becton Dickinson, Franklin Lakes, N.J.) to prevent blood clotting, and plasma can then be obtained from whole blood through centrifugation. Serum may be obtained with or without centrifugation-following blood clotting. If centrifugation is used then it is typically, though not exclusively, conducted at an appropriate speed, e.g., 1,500-3,000 times g. Plasma or serum may be subjected to additional centrifugation steps before being transferred to a fresh tube for nucleic acid extraction. In addition to the acellular portion of the whole blood, nucleic acid may also be recovered from the cellular fraction, enriched in the buffy coat portion, which can be obtained following centrifugation of a whole blood sample from the subject and removal of the plasma.

A sample may be a tumor nucleic acid sample (i.e., a nucleic acid sample isolated from a tumor). The term “tumor” generally refers to neoplastic cell growth and proliferation, whether malignant or benign, and may include pre-cancerous and cancerous cells and tissues. The terms “cancer” and “cancerous” generally refer to the physiological condition in mammals that is typically characterized by unregulated cell growth/proliferation. Examples of cancer include, but are not limited to, carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, various types of head and neck cancer, and the like.

A sample may be heterogeneous. For example, a sample may include more than one cell type and/or one or more nucleic acid species. In some instances, a sample may include (i) fetal cells and maternal cells, (ii) cancer cells and non-cancer cells, and/or (iii) pathogenic cells and host cells. In some instances, a sample may include (i) cancer and non-cancer nucleic acid, (ii) pathogen and host nucleic acid, (iii) fetal derived and maternal derived nucleic acid, and/or more generally, (iv) mutated and wild-type nucleic acid. In some instances, a sample may include a minority nucleic acid species and a majority nucleic acid species, as described in further detail below. In some instances, a sample may include cells and/or nucleic acid from a single subject or may include cells and/or nucleic acid from multiple subjects.

Nucleic Acid

Provided herein are methods and compositions for processing and/or analyzing nucleic acid. The terms nucleic acid(s), nucleic acid molecule(s), nucleic acid fragment(s), target nucleic acid(s), nucleic acid template(s), template nucleic acid(s), nucleic acid target(s), target nucleic acid(s), polynucleotide(s), polynucleotide fragment(s), target polynucleotide(s), polynucleotide target(s), and the like may be used interchangeably throughout the disclosure. The terms refer to nucleic acids of any composition from, such as DNA (e.g., complementary DNA (cDNA; synthesized from any RNA or DNA of interest), genomic DNA (gDNA), genomic DNA fragments, mitochondrial DNA (mtDNA), recombinant DNA (e.g., plasmid DNA), and the like), RNA (e.g., message RNA (mRNA), short inhibitory RNA (siRNA), ribosomal RNA (rRNA), transfer RNA (tRNA), microRNA, transacting small interfering RNA (ta-siRNA), natural small interfering RNA (nat-siRNA), small nucleolar RNA (snoRNA), small nuclear RNA (snRNA), long non-coding RNA (lncRNA), non-coding RNA (ncRNA), transfer-messenger RNA (tmRNA), precursor messenger RNA (pre-mRNA), small Cajal body-specific RNA (scaRNA), piwi-interacting RNA (piRNA), endoribonuclease-prepared siRNA (esiRNA), small temporal RNA (stRNA), signal recognition RNA, telomere RNA, RNA highly expressed by a fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid may be, or may be from, a plasmid, phage, virus, bacterium, autonomously replicating sequence (ARS), mitochondria, centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense,” “plus” strand or “minus” strand, “forward” reading frame or “reverse” reading frame) and double-stranded polynucleotides. The term “gene” refers to a section of DNA involved in producing a polypeptide chain; and generally includes regions preceding and following the coding region (leader and trailer) involved in the transcription/translation of the gene product and the regulation of the transcription/translation, as well as intervening sequences (introns) between individual coding regions (exons). A nucleotide or base generally refers to the purine and pyrimidine molecular units of nucleic acid (e.g., adenine (A), thymine (T), guanine (G), and cytosine (C)). For RNA, the base thymine is replaced with uracil. Nucleic acid length or size may be expressed as a number of bases.

Target nucleic acids may be any nucleic acids of interest. Nucleic acids may be polymers of any length composed of deoxyribonucleotides (i.e., DNA bases), ribonucleotides (i.e., RNA bases), or combinations thereof, e.g., 10 bases or longer, 20 bases or longer, 50 bases or longer, 100 bases or longer, 200 bases or longer, 300 bases or longer, 400 bases or longer, 500 bases or longer, 1000 bases or longer, 2000 bases or longer, 3000 bases or longer, 4000 bases or longer, 5000 bases or longer. In certain aspects, nucleic acids are polymers composed of deoxyribonucleotides (i.e., DNA bases), ribonucleotides (i.e., RNA bases), or combinations thereof, e.g., 10 bases or less, 20 bases or less, 50 bases or less, 100 bases or less, 200 bases or less, 300 bases or less, 400 bases or less, 500 bases or less, 1000 bases or less, 2000 bases or less, 3000 bases or less, 4000 bases or less, or 5000 bases or less.

Nucleic acid may be single or double stranded. Single stranded DNA (ssDNA), for example, can be generated by denaturing double stranded DNA by heating or by treatment with alkali, for example. Accordingly, in some embodiments, ssDNA is derived from double-stranded DNA (dsDNA). In some embodiments, a method herein comprises prior to combining a nucleic acid composition comprising dsDNA with the scaffold adapters herein, or components thereof, denaturing the dsDNA, thereby generating ssDNA.

In certain embodiments, nucleic acid is in a D-loop structure, formed by strand invasion of a duplex DNA molecule by an oligonucleotide or a DNA-like molecule such as peptide nucleic acid (PNA). D loop formation can be facilitated by addition of E. coli RecA protein and/or by alteration of salt concentration, for example, using methods known in the art.

Nucleic acid (e.g., nucleic acid targets, single-stranded nucleic acid (ssNA), oligonucleotides, overhangs, scaffold polynucleotides and hybridization regions thereof (e.g., ssNA hybridization region, oligonucleotide hybridization region)) may be described herein as being complementary to another nucleic acid, having a complementarity region, being capable of hybridizing to another nucleic acid, or having a hybridization region. The terms “complementary” or “complementarity” or “hybridization” generally refer to a nucleotide sequence that base-pairs by non-covalent bonds to a region of a nucleic acid (e.g., the nucleotide sequence of an ssNA hybridization region that hybridizes to the terminal region of an ssNA fragment, and the nucleotide sequence of an oligonucleotide hybridization region that hybridizes to an oligonucleotide component of a scaffold adapter). In the canonical Watson-Crick base pairing, adenine (A) forms a base pair with thymine (T), and guanine (G) pairs with cytosine (C) in DNA. In RNA, thymine (T) is replaced by uracil (U). As such, A is complementary to T and G is complementary to C. In RNA, A is complementary to U and vice versa. In a DNA-RNA duplex, A (in a DNA strand) is complementary to U (in an RNA strand). In some embodiments, one or more thymine (T) bases are replaced by uracil (U) in a scaffold adapter, or a component thereof, and is/are complementary to adenine (A). Typically, “complementary” or “complementarity” or “capable of hybridizing” refer to a nucleotide sequence that is at least partially complementary. These terms may also encompass duplexes that are fully complementary such that every nucleotide in one strand is complementary or hybridizes to every nucleotide in the other strand in corresponding positions.

In certain instances, a nucleotide sequence may be partially complementary to a target, in which not all nucleotides are complementary to every nucleotide in the target nucleic acid in all the corresponding positions. For example, an ssNA hybridization region may be perfectly (i.e., 100%) complementary to a target ssNA terminal region, or an ssNA hybridization region may share some degree of complementarity which is less than perfect (e.g., 70%, 75%, 85%, 90%, 95%, 99%). In another example, an oligonucleotide hybridization region may be perfectly (i.e., 100%) complementary to an oligonucleotide, or an oligonucleotide hybridization region may share some degree of complementarity which is less than perfect (e.g., 70%, 75%, 85%, 90%, 95%, 99%).

The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=# of identical positions/total # of positions×100). When a position in one sequence is occupied by the same nucleotide as the corresponding position in the other sequence, then the molecules are identical at that position.

In some embodiments, nucleic acids in a mixture of nucleic acids are analyzed. A mixture of nucleic acids can comprise two or more nucleic acid species having the same or different nucleotide sequences, different lengths, different origins (e.g., genomic origins, fetal vs. maternal origins, cell or tissue origins, cancer vs. non-cancer origin, tumor vs. non-tumor origin, host vs. pathogen, host vs. transplant, host vs. microbiome, sample origins, subject origins, and the like), different overhang lengths, different overhang types (e.g., 5′ overhangs, 3′ overhangs, no overhangs), or combinations thereof. In some embodiments, a mixture of nucleic acids comprises single-stranded nucleic acid and double-stranded nucleic acid. In some embodiment, a mixture of nucleic acids comprises DNA and RNA. In some embodiment, a mixture of nucleic acids comprises ribosomal RNA (rRNA) and messenger RNA (mRNA). Nucleic acid provided for processes described herein may contain nucleic acid from one sample or from two or more samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more samples).

In some embodiments, target nucleic acids (e.g., ssNAs) comprise degraded DNA. Degraded DNA may be referred to as low-quality DNA or highly degraded DNA. Degraded DNA may be highly fragmented, and may include damage such as base analogs and abasic sites subject to miscoding lesions and/or intermolecular crosslinking. For example, sequencing errors resulting from deamination of cytosine residues may be present in certain sequences obtained from degraded DNA (e.g., miscoding of C to T and G to A). In some embodiments, target nucleic acids (e.g., ssNAs) are derived from nicked double-stranded nucleic acid fragments. Nicked double-stranded nucleic acid fragments may be denatured (e.g., heat denatured) to generate ssNA fragments.

Nucleic acid may be derived from one or more sources (e.g., biological sample, blood, cells, serum, plasma, buffy coat, urine, lymphatic fluid, skin, hair, soil, and the like) by methods known in the art. Any suitable method can be used for isolating, extracting and/or purifying DNA from a biological sample (e.g., from blood or a blood product), non-limiting examples of which include methods of DNA preparation (e.g., described by Sambrook and Russell, Molecular Cloning: A Laboratory Manual 3d ed., 2001), various commercially available reagents or kits, such as DNeasy®, RNeasy®, QIAprep®, QIAquick®, and QIAamp® (e.g., QIAamp® Circulating Nucleic Acid Kit, QiaAmp® DNA Mini Kit or QiaAmp® DNA Blood Mini Kit) nucleic acid isolation/purification kits by Qiagen, Inc. (Germantown, Md); GenomicPrep™ Blood DNA Isolation Kit (Promega, Madison, Wis.); GFX™ Genomic Blood DNA Purification Kit (Amersham, Piscataway, N.J.); DNAzol©, ChargeSwitch®, Purelink®, GeneCatcher® nucleic acid isolation/purification kits by Life Technologies, Inc. (Carlsbad, CA); NucleoMag®, NucleoSpin®, and NucleoBond® nucleic acid isolation/purification kits by Clontech Laboratories, Inc. (Mountain View, CA); the like or combinations thereof. In certain aspects, the nucleic acid is isolated from a fixed biological sample, e.g., formalin-fixed, paraffin-embedded (FFPE) tissue. Genomic DNA from FFPE tissue may be isolated using commercially available kits—such as the AllPrep® DNA/RNA FFPE kit by Qiagen, Inc. (Germantown, Md), the RecoverAll® Total Nucleic Acid Isolation kit for FFPE by Life Technologies, Inc. (Carlsbad, CA), and the NucleoSpin® FFPE kits by Clontech Laboratories, Inc. (Mountain View, CA).

In some embodiments, nucleic acid is extracted from cells using a cell lysis procedure. Cell lysis procedures and reagents are known in the art and may generally be performed by chemical (e.g., detergent, hypotonic solutions, enzymatic procedures, and the like, or combination thereof), physical (e.g., French press, sonication, and the like), or electrolytic lysis methods. Any suitable lysis procedure can be utilized. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. In some instances, a high salt and/or an alkaline lysis procedure may be utilized. In some instances, a lysis procedure may include a lysis step with EDTA/Proteinase K, a binding buffer step with high amount of salts (e.g., guanidinium chloride (GuHCl), sodium acetate) and isopropanol, and binding DNA in this solution to silica-based column. In some instances, a lysis protocol includes certain procedures described in Dabney et al., Proceedings of the National Academy of Sciences 110, no. 39 (2013): 15758-15763.

Nucleic acids can include extracellular nucleic acid in certain embodiments. The term “extracellular nucleic acid” as used herein can refer to nucleic acid isolated from a source having substantially no cells and also is referred to as “cell-free” nucleic acid (cell-free DNA, cell-free RNA, or both), “circulating cell-free nucleic acid” (e.g., CCF fragments, ccfDNA) and/or “cell-free circulating nucleic acid.” Extracellular nucleic acid can be present in and obtained from blood (e.g., from the blood of a human subject). Extracellular nucleic acid often includes no detectable cells and may contain cellular elements or cellular remnants. Non-limiting examples of acellular sources for extracellular nucleic acid are blood, blood plasma, blood serum and urine. In certain aspects, cell-free nucleic acid is obtained from a body fluid sample chosen from whole blood, blood plasma, blood serum, amniotic fluid, saliva, urine, pleural effusion, bronchial lavage, bronchial aspirates, breast milk, colostrum, tears, seminal fluid, peritoneal fluid, pleural effusion, and stool. As used herein, the term “obtain cell-free circulating sample nucleic acid” includes obtaining a sample directly (e.g., collecting a sample, e.g., a test sample) or obtaining a sample from another who has collected a sample. Extracellular nucleic acid may be a product of cellular secretion and/or nucleic acid release (e.g., DNA release). Extracellular nucleic acid may be a product of any form of cell death, for example. In some instances, extracellular nucleic acid is a product of any form of type I or type II cell death, including mitotic, oncotic, toxic, ischemic, and the like and combinations thereof. Without being limited by theory, extracellular nucleic acid may be a product of cell apoptosis and cell breakdown, which provides basis for extracellular nucleic acid often having a series of lengths across a spectrum (e.g., a “ladder”). In some instances, extracellular nucleic acid is a product of cell necrosis, necropoptosis, oncosis, entosis, pyrotosis, and the like and combinations thereof. In some embodiments, sample nucleic acid from a test subject is circulating cell-free nucleic acid. In some embodiments, circulating cell free nucleic acid is from blood plasma or blood serum from a test subject. In some aspects, cell-free nucleic acid is degraded. In some embodiments, cell-free nucleic acid comprises cell-free fetal nucleic acid (e.g., cell-free fetal DNA). In certain aspects, cell-free nucleic acid comprises circulating cancer nucleic acid (e.g., cancer DNA). In certain aspects, cell-free nucleic acid comprises circulating tumor nucleic acid (e.g., tumor DNA). In some embodiments, cell-free nucleic acid comprises infectious agent nucleic acid (e.g., pathogen DNA). In some embodiments, cell-free nucleic acid comprises nucleic acid (e.g., DNA) from a transplant. In some embodiments, cell-free nucleic acid comprises nucleic acid (e.g., DNA) from a microbiome (e.g., microbiome of gut, microbiome of blood, microbiome of mouth, microbiome of spinal fluid, microbiome of feces).

Cell-free DNA (cfDNA) may originate from degraded sources and often provides limiting amounts of DNA when extracted. Methods described herein for generating single-stranded DNA (ssDNA) libraries are able to capture a larger amount of short DNA fragments from cfDNA. cfDNA from cancer samples, for example, tends to have a higher population of short fragments. In certain instances, short fragments in cfDNA may be enriched for fragments originating from transcription factors rather than nucleosomes.

Extracellular nucleic acid can include different nucleic acid species, and therefore is referred to herein as “heterogeneous” in certain embodiments. For example, blood serum or plasma from a person having a tumor or cancer can include nucleic acid from tumor cells or cancer cells (e.g., neoplasia) and nucleic acid from non-tumor cells or non-cancer cells. In another example, blood serum or plasma from a pregnant female can include maternal nucleic acid and fetal nucleic acid. In another example, blood serum or plasma from a patient having an infection or infectious disease can include host nucleic acid and infectious agent or pathogen nucleic acid. In another example, a sample from a subject having received a transplant can include host nucleic acid and nucleic acid from the donor organ or tissue. In some instances, cancer nucleic acid, tumor nucleic acid, fetal nucleic acid, pathogen nucleic acid, or transplant nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is cancer, tumor, fetal, pathogen, transplant, or microbiome nucleic acid). In another example, heterogeneous nucleic acid may include nucleic acid from two or more subjects (e.g., a sample from a crime scene).

At least two different nucleic acid species can exist in different amounts in extracellular nucleic acid and sometimes are referred to as minority species and majority species. In certain instances, a minority species of nucleic acid is from an affected cell type (e.g., cancer cell, wasting cell, cell attacked by immune system). In certain embodiments, a genetic variation or genetic alteration (e.g., copy number alteration, copy number variation, single nucleotide alteration, single nucleotide variation, chromosome alteration, and/or translocation) is determined for a minority nucleic acid species. In certain embodiments, a genetic variation or genetic alteration is determined for a majority nucleic acid species. Generally, it is not intended that the terms “minority” or “majority” be rigidly defined in any respect. In one aspect, a nucleic acid that is considered “minority,” for example, can have an abundance of at least about 0.1% of the total nucleic acid in a sample to less than 50% of the total nucleic acid in a sample. In some embodiments, a minority nucleic acid can have an abundance of at least about 1% of the total nucleic acid in a sample to about 40% of the total nucleic acid in a sample. In some embodiments, a minority nucleic acid can have an abundance of at least about 2% of the total nucleic acid in a sample to about 30% of the total nucleic acid in a sample. In some embodiments, a minority nucleic acid can have an abundance of at least about 3% of the total nucleic acid in a sample to about 25% of the total nucleic acid in a sample. For example, a minority nucleic acid can have an abundance of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% of the total nucleic acid in a sample. In some instances, a minority species of extracellular nucleic acid sometimes is about 1% to about 40% of the overall nucleic acid (e.g., about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40% of the nucleic acid is minority species nucleic acid). In some embodiments, the minority nucleic acid is extracellular DNA. In some embodiments, the minority nucleic acid is extracellular DNA from apoptotic tissue. In some embodiments, the minority nucleic acid is extracellular DNA from tissue where some cells therein underwent apoptosis. In some embodiments, the minority nucleic acid is extracellular DNA from necrotic tissue. In some embodiments, the minority nucleic acid is extracellular DNA from tissue where some cells therein underwent necrosis. Necrosis may refer to a post-mortem process following cell death, in certain instances. In some embodiments, the minority nucleic acid is extracellular DNA from tissue affected by a cell proliferative disorder (e.g., cancer). In some embodiments, the minority nucleic acid is extracellular DNA from a tumor cell. In some embodiments, the minority nucleic acid is extracellular fetal DNA. In some embodiments, the minority nucleic acid is extracellular DNA from a pathogen. In some embodiments, the minority nucleic acid is extracellular DNA from a transplant. In some embodiments, the minority nucleic acid is extracellular DNA from a microbiome.

In another aspect, a nucleic acid that is considered “majority,” for example, can have an abundance greater than 50% of the total nucleic acid in a sample to about 99.9% of the total nucleic acid in a sample. In some embodiments, a majority nucleic acid can have an abundance of at least about 60% of the total nucleic acid in a sample to about 99% of the total nucleic acid in a sample. In some embodiments, a majority nucleic acid can have an abundance of at least about 70% of the total nucleic acid in a sample to about 98% of the total nucleic acid in a sample. In some embodiments, a majority nucleic acid can have an abundance of at least about 75% of the total nucleic acid in a sample to about 97% of the total nucleic acid in a sample. For example, a majority nucleic acid can have an abundance of at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total nucleic acid in a sample. In some embodiments, the majority nucleic acid is extracellular DNA. In some embodiments, the majority nucleic acid is extracellular maternal DNA. In some embodiments, the majority nucleic acid is DNA from healthy tissue. In some embodiments, the majority nucleic acid is DNA from non-tumor cells. In some embodiments, the majority nucleic acid is DNA from host cells.

In some embodiments, a minority species of extracellular nucleic acid is of a length of about 500 base pairs or less (e.g., about 80, 85, 90, 91, 92, 93, 94, 95, 96, 97, 98, 99 or 100% of minority species nucleic acid is of a length of about 500 base pairs or less). In some embodiments, a minority species of extracellular nucleic acid is of a length of about 300 base pairs or less (e.g., about 80, 85, 90, 91, 92, 93, 94, 95, 96, 97, 98, 99 or 100% of minority species nucleic acid is of a length of about 300 base pairs or less). In some embodiments, a minority species of extracellular nucleic acid is of a length of about 250 base pairs or less (e.g., about 80, 85, 90, 91, 92, 93, 94, 95, 96, 97, 98, 99 or 100% of minority species nucleic acid is of a length of about 250 base pairs or less). In some embodiments, a minority species of extracellular nucleic acid is of a length of about 200 base pairs or less (e.g., about 80, 85, 90, 91, 92, 93, 94, 95, 96, 97, 98, 99 or 100% of minority species nucleic acid is of a length of about 200 base pairs or less). In some embodiments, a minority species of extracellular nucleic acid is of a length of about 150 base pairs or less (e.g., about 80, 85, 90, 91, 92, 93, 94, 95, 96, 97, 98, 99 or 100% of minority species nucleic acid is of a length of about 150 base pairs or less). In some embodiments, a minority species of extracellular nucleic acid is of a length of about 100 base pairs or less (e.g., about 80, 85, 90, 91, 92, 93, 94, 95, 96, 97, 98, 99 or 100% of minority species nucleic acid is of a length of about 100 base pairs or less). In some embodiments, a minority species of extracellular nucleic acid is of a length of about 50 base pairs or less (e.g., about 80, 85, 90, 91, 92, 93, 94, 95, 96, 97, 98, 99 or 100% of minority species nucleic acid is of a length of about 50 base pairs or less).

Nucleic acid may be provided for conducting methods described herein with or without processing of the sample(s) containing the nucleic acid. In some embodiments, nucleic acid is provided for conducting methods described herein after processing of the sample(s) containing the nucleic acid. For example, a nucleic acid can be extracted, isolated, purified, partially purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a subject (e.g., a human subject). An isolated nucleic acid can be provided with fewer non-nucleic acid components (e.g., protein, lipid) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” as used herein can refer to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components. The term “purified” as used herein can refer to a nucleic acid provided that contains fewer nucleic acid species than in the sample source from which the nucleic acid is derived. A composition comprising purified nucleic acid may be about 90%, 91%, 92%, 93%, 94%, 95%, 96%, 97%, 98%, 99% or greater than 99% free of other nucleic acid species. For example, fetal nucleic acid can be purified from a mixture comprising maternal and fetal nucleic acid. In certain examples, small fragments of nucleic acid (e.g., 30 to 500 bp fragments) can be purified, or partially purified, from a mixture comprising nucleic acid fragments of different lengths. In certain examples, nucleosomes comprising smaller fragments of nucleic acid can be purified from a mixture of larger nucleosome complexes comprising larger fragments of nucleic acid. In certain examples, larger nucleosome complexes comprising larger fragments of nucleic acid can be purified from nucleosomes comprising smaller fragments of nucleic acid. In certain examples, small fragments of fetal nucleic acid (e.g., 30 to 500 bp fragments) can be purified, or partially purified, from a mixture comprising both fetal and maternal nucleic acid fragments. In certain examples, nucleosomes comprising smaller fragments of fetal nucleic acid can be purified from a mixture of larger nucleosome complexes comprising larger fragments of maternal nucleic acid. In certain examples, cancer cell nucleic acid can be purified from a mixture comprising cancer cell and non-cancer cell nucleic acid. In certain examples, nucleosomes comprising small fragments of cancer cell nucleic acid can be purified from a mixture of larger nucleosome complexes comprising larger fragments of non-cancer nucleic acid. In some embodiments, nucleic acid is provided for conducting methods described herein without prior processing of the sample(s) containing the nucleic acid. For example, nucleic acid may be analyzed directly from a sample without prior extraction, purification, partial purification, and/or amplification.

Nucleic acids may be amplified under amplification conditions. The term “amplified” or “amplification” or “amplification conditions” as used herein refers to subjecting a target nucleic acid (e.g., ssNA) in a sample or a nucleic acid product generated by a method herein to a process that linearly or exponentially generates amplicon nucleic acids having the same or substantially the same nucleotide sequence as the target nucleic acid (e.g., ssNA), or part thereof. In certain embodiments, the term “amplified” or “amplification” or “amplification conditions” refers to a method that comprises a polymerase chain reaction (PCR). In certain instances, an amplified product can contain one or more nucleotides more than the amplified nucleotide region of a nucleic acid template sequence (e.g., a primer can contain “extra” nucleotides such as a transcriptional initiation sequence, in addition to nucleotides complementary to a nucleic acid template gene molecule, resulting in an amplified product containing “extra” nucleotides or nucleotides not corresponding to the amplified nucleotide region of the nucleic acid template gene molecule).

Nucleic acid also may be exposed to a process that modifies certain nucleotides in the nucleic acid before providing nucleic acid for a method described herein. A process that selectively modifies nucleic acid based upon the methylation state of nucleotides therein can be applied to nucleic acid, for example. In addition, conditions such as high temperature, ultraviolet radiation, x-radiation, can induce changes in the sequence of a nucleic acid molecule. Nucleic acid may be provided in any suitable form useful for conducting a sequence analysis.

In some embodiments, target nucleic acids (e.g., ssNAs) are not modified in prior to combining with the scaffold adapters herein, or components thereof. In some embodiments, target nucleic acids (e.g., ssNAs) are not modified in length prior to combining with the scaffold adapters herein, or components thereof. In this context, “not modified” means that target nucleic acids are isolated from a sample and then combined with scaffold adapters, or components thereof, without modifying the length or the composition of the target nucleic acids. For example, target nucleic acids (e.g., ssNAs) may not be shortened (e.g., they are not contacted with a restriction enzyme or nuclease or physical condition that reduces length (e.g., shearing condition, cleavage condition)) and may not be increased in length by one or more nucleotides (e.g., ends are not filled in at overhangs; no nucleotides are added to the ends). Adding a phosphate or chemically reactive group to one or both ends of a target nucleic acid (e.g., ssNA) generally is not considered modifying the nucleic acid or modifying the length of the nucleic acid. Denaturing a double-stranded nucleic acid (dsNA) fragment to generate an ssNA fragment generally is not considered modifying the nucleic acid or modifying the length of the nucleic acid.

In some embodiments, one or both native ends of target nucleic acids (e.g., ssNAs) are present when the ssNA is combined with the scaffold adapters herein, or components thereof. Native ends generally refer to unmodified ends of a nucleic acid fragment. In some embodiments, native ends of target nucleic acids (e.g., ssNAs) are not modified in length prior to combining with the scaffold adapters herein, or components thereof. In this context, “not modified” means that target nucleic acids are isolated from a sample and then combined with scaffold adapters, or components thereof, without modifying the length of the native ends of target nucleic acids. For example, target nucleic acids (e.g., ssNAs) are not shortened (e.g., they are not contacted with a restriction enzyme or nuclease or physical condition that reduces length (e.g., shearing condition, cleavage condition) to generate non-native ends) and are not increased in length by one or more nucleotides (e.g., native ends are not filled in at overhangs; no nucleotides are added to the native ends). Adding a phosphate or chemically reactive group to one or both native ends of a target nucleic acid generally is not considered modifying the length of the nucleic acid.

In some embodiments, target nucleic acids (e.g., ssNAs) are not contacting with a cleavage agent (e.g., endonuclease, exonuclease, restriction enzyme) and/or a polymerase prior to combining with the scaffold adapters herein, or components thereof. In some embodiments, target nucleic acids are not subjected to mechanical shearing (e.g., ultrasonication (e.g., Adaptive Focused Acoustics™ (AFA) process by Covaris)) prior to combining with the scaffold adapters herein, or components thereof. In some embodiments, target nucleic acids are not contacting with an exonuclease (e.g., DNAse) prior to combining with the scaffold adapters herein, or components thereof. In some embodiments, target nucleic acids are not amplified prior to combining with the scaffold adapters herein, or components thereof. In some embodiments, target nucleic acids are not attached to a solid support prior to combining with the scaffold adapters herein, or components thereof. In some embodiments, target nucleic acids are not conjugated to another molecule prior to combining with the scaffold adapters herein, or components thereof. In some embodiments, target nucleic acids are not cloned into a vector prior to combining with the scaffold adapters herein, or components thereof. In some embodiments, target nucleic acids may be subjected to dephosphorylation prior to combining with the scaffold adapters herein, or components thereof. In some embodiments, target nucleic acids may be subjected to phosphorylation prior to combining with the scaffold adapters herein, or components thereof.

In some embodiments, combining target nucleic acids (e.g., ssNAs) with the scaffold adapters herein, or components thereof, comprises isolating the target nucleic acids, and combining the isolated target nucleic acids with the scaffold adapters herein, or components thereof. In some embodiments, combining target nucleic acids with the scaffold adapters herein, or components thereof, comprises isolating the target nucleic acids, phosphorylating the isolated target nucleic acids, and combining the phosphorylated target nucleic acids with the scaffold adapters herein, or components thereof. In some embodiments, combining target nucleic acids with the scaffold adapters herein, or components thereof, comprises isolating the target nucleic acids, dephosphorylating the scaffold adapters herein, or components thereof, and combining the isolated target nucleic acids with the dephosphorylated scaffold adapters herein, or dephosphorylated components thereof. In some embodiments, combining target nucleic acids with the scaffold adapters herein, or components thereof, comprises isolating the target nucleic acids, dephosphorylating the isolated target nucleic acids, phosphorylating the dephosphorylated target nucleic acids, and combining the phosphorylated target nucleic acids with the scaffold adapters herein, or components thereof. In some embodiments, combining target nucleic acids with the scaffold adapters herein, or components thereof, comprises isolating the target nucleic acids, dephosphorylating the isolated target nucleic acids, phosphorylating the dephosphorylated target nucleic acids, dephosphorylating the scaffold adapters, or components thereof, and combining the phosphorylated target nucleic acids with the dephosphorylated scaffold adapters herein, or dephosphorylated components thereof.

In some embodiments, combining target nucleic acids (e.g., ssNAs) with the scaffold adapters herein, or components thereof, consists of isolating the target nucleic acids, and combining the isolated target nucleic acids with the scaffold adapters herein, or components thereof. In some embodiments, combining target nucleic acids with the scaffold adapters herein, or components thereof, consists of isolating the target nucleic acids, phosphorylating the isolated target nucleic acids, and combining the phosphorylated target nucleic acids with the scaffold adapters herein, or components thereof. In some embodiments, combining target nucleic acids with the scaffold adapters herein, or components thereof, consists of isolating the target nucleic acids, dephosphorylating the scaffold adapters, or components thereof, and combining the isolated target nucleic acids with the dephosphorylated scaffold adapters herein, or dephosphorylated components thereof. In some embodiments, combining target nucleic acids with the scaffold adapters herein, or components thereof, consists of isolating the target nucleic acids, dephosphorylating the isolated target nucleic acids, phosphorylating the dephosphorylated target nucleic acids, and combining the phosphorylated target nucleic acids with the scaffold adapters herein, or components thereof. In some embodiments, combining target nucleic acids with the scaffold adapters herein, or components thereof, consists of isolating the target nucleic acids, dephosphorylating the isolated target nucleic acids, phosphorylating the dephosphorylated target nucleic acids, dephosphorylating the scaffold adapters, or components thereof, and combining the phosphorylated target nucleic acids with the dephosphorylated scaffold adapters herein, or dephosphorylated components thereof.

Single-Stranded Nucleic Acid Provided herein are methods and compositions for capturing single-stranded nucleic acid (ssNA) using specialized adapters (e.g., for generating a sequencing library). Single-stranded nucleic acid or ssNA generally refers to a collection of polynucleotides which are single-stranded (i.e., not hybridized intermolecularly or intramolecularly) over 70% or more of their length. In some embodiments, ssNA is single-stranded over 75% or more, 80% or more, 85% or more, 90% or more, 95% or more, or 99% or more, of the length of the polynucleotides. In certain aspects, the ssNA is single-stranded over the entire length of the polynucleotides. Single-stranded nucleic acid may be referred to herein as target nucleic acid.

ssNA may include single-stranded deoxyribonucleic acid (ssDNA). In some embodiments, ssDNA includes, but is not limited to, ssDNA derived from double-stranded DNA (dsDNA). For example, ssDNA may be derived from double-stranded DNA which is denatured (e.g., heat denatured and/or chemically denatured) to produce ssDNA. In some embodiments, a method herein comprises, prior to combining ssDNA with scaffold adapters described herein, or components thereof, generating the ssDNA by denaturing dsDNA.

In some embodiments, ssNA includes single-stranded ribonucleic acid (ssRNA). RNA may include, for example, messenger RNA (mRNA), microRNA (miRNA), small interfering RNA (siRNA), transacting small interfering RNA (ta-siRNA), natural small interfering RNA (nat-siRNA), ribosomal RNA (rRNA), transfer RNA (tRNA), small nucleolar RNA (snoRNA), small nuclear RNA (snRNA), long non-coding RNA (lncRNA), non-coding RNA (ncRNA), transfer-messenger RNA (tmRNA), precursor messenger RNA (pre-mRNA), small Cajal body-specific RNA (scaRNA), piwi-interacting RNA (piRNA), endoribonuclease prepared siRNA (esiRNA), small temporal RNA (stRNA), signal recognition RNA, telomere RNA, ribozyme, or a combination thereof. In some embodiments, when the ssNA is ssRNA, the ssRNA is mRNA. In some embodiments, ssNA includes single stranded complementary DNA (cDNA).

In some embodiments, a method herein comprises contacting ssNA with a single-stranded nucleic acid binding agent. In some embodiments, a method herein comprises contacting ssNA with single-stranded nucleic acid binding protein (SSB) to produce SSB-bound ssNA. In some embodiments, a method herein comprises contacting ssDNA with single-stranded nucleic acid binding protein (SSB) to produce SSB-bound ssDNA. In some embodiments, a method herein comprises contacting ssRNA with single-stranded nucleic acid binding protein (SSB) to produce SSB-bound ssRNA. SSB generally binds in a cooperative manner to ssNA and typically does not bind well to double-stranded nucleic acid (dsNA). Upon binding ssDNA, SSB destabilizes helical duplexes. SSBs may be prokaryotic SSB (e.g., bacterial or archaeal SSB) or eukaryotic SSB. Examples of SSBs may include E. coli SSB, E. coli RecA, Extreme Thermostable Single-Stranded DNA Binding Protein (ET SSB), Thermus thermophilus (Tth) RecA, T4 Gene 32 Protein, replication protein A (RPA—a eukaryotic SSB), and the like. ET SSB, Tth RecA, E. coli RecA, T4 Gene 32 Protein, as well buffers and detailed protocols for preparing SSB-bound ssNA using such SSBs are commercially available (e.g., New England Biolabs, Inc. (Ipswich, MA)).

In some embodiments, a method herein does not comprise contacting ssNA with single-stranded nucleic acid binding protein (SSB) to produce SSB-bound ssNA. Accordingly, a method herein may omit the step of producing SSB-bound ssNA. For example, a method herein may comprise combining ssNA with scaffold adapters described herein, or components thereof, without contacting the ssNA with SSB. In such instances, a method herein may be referred to an “SSB-free” method for producing a nucleic acid library. Certain SSB-free methods described herein may produce libraries having parameters similar to parameters for libraries prepared using SSB, as shown in the Drawings and discussed in the Examples. In some embodiments, a method herein comprises contacting ssNA with a single-stranded nucleic acid binding agent other than SSB. Such single-stranded nucleic acid binding agents can stably bind single stranded nucleic acids, can prevent or reduce formation of nucleic acid duplexes, can still allow the bound nucleic acids to be ligated or otherwise terminally modified, and can be thermostable. Example single-stranded nucleic acid binding agents include but are not limited to topoisomerases, helicases, domains thereof, and fusion proteins comprising domains thereof.

In some embodiments, a method herein comprises combining a nucleic acid composition comprising single-stranded nucleic acid (ssNA) with scaffold adapters described herein, or components thereof. In some embodiments, a method herein comprises combining a nucleic acid composition consisting of single-stranded nucleic acid (ssNA) with scaffold adapters described herein, or components thereof. In some embodiments, a method herein comprises combining a nucleic acid composition consisting essentially of single-stranded nucleic acid (ssNA) with scaffold adapters described herein, or components thereof. A nucleic acid composition “consisting essentially of” single-stranded nucleic acid (ssNA) generally includes ssNA and no additional protein or nucleic acid components. For example, a nucleic acid composition “consisting essentially of” single-stranded nucleic acid (ssNA) may exclude double-stranded nucleic acid (dsNA) or may include a low percentage of dsNA (e.g., less than 10% dsNA, less than 5% dsNA, less than 1% dsNA). A nucleic acid composition “consisting essentially of” single-stranded nucleic acid (ssNA) may exclude proteins. For example, a nucleic acid composition “consisting essentially of” single-stranded nucleic acid (ssNA) may exclude single-stranded binding proteins (SSBs) or other proteins useful for stabilizing ssNA. A nucleic acid composition “consisting essentially of” single-stranded nucleic acid (ssNA) may include chemical components typically present in nucleic acid compositions such as buffers, salts, alcohols, crowding agents (e.g., PEG), and the like; and may include residual components (e.g., nucleic acids, proteins, cell membrane components) from the nucleic acid source (e.g., sample) or nucleic acid extraction. A nucleic acid composition “consisting essentially of” single-stranded nucleic acid (ssNA) may include ssNA fragments having one or more phosphates (e.g., a terminal phosphate, a 5′ terminal phosphate). A nucleic acid composition “consisting essentially of” single-stranded nucleic acid (ssNA) may include ssNA fragments comprising one or more modified nucleotides.

Enriching Nucleic Acids

In some embodiments, nucleic acid (e.g., extracellular nucleic acid) is enriched or relatively enriched for a subpopulation or species of nucleic acid. Nucleic acid subpopulations can include, for example, fetal nucleic acid, maternal nucleic acid, cancer nucleic acid, tumor nucleic acid, patient nucleic acid, host nucleic acid, pathogen nucleic acid, transplant nucleic acid, microbiome nucleic acid, nucleic acid comprising fragments of a particular length or range of lengths, or nucleic acid from a particular genome region (e.g., single chromosome, set of chromosomes, and/or certain chromosome regions). Such enriched samples can be used in conjunction with a method provided herein. Thus, in certain embodiments, methods of the technology comprise an additional step of enriching for a subpopulation of nucleic acid in a sample. In certain embodiments, nucleic acid from normal tissue (e.g., non-cancer cells, host cells) is selectively removed (partially, substantially, almost completely or completely) from the sample. In certain embodiments, maternal nucleic acid is selectively removed (partially, substantially, almost completely or completely) from the sample. In certain embodiments, enriching for a particular low copy number species nucleic acid (e.g., cancer, tumor, fetal, pathogen, transplant, microbiome nucleic acid) may improve quantitative sensitivity. Methods for enriching a sample for a particular species of nucleic acid are described, for example, in U.S. Pat. No. 6,927,028, International Patent Application Publication No. WO2007/140417, International Patent Application Publication No. WO2007/147063, International Patent Application Publication No. WO2009/032779, International Patent Application Publication No. WO2009/032781, International Patent Application Publication No. WO2010/033639, International Patent Application Publication No. WO2011/034631, International Patent Application Publication No. WO2006/056480, and International Patent Application Publication No. WO2011/143659, the entire content of each is incorporated herein by reference, including all text, tables, equations and drawings.

In some embodiments, nucleic acid is enriched for certain target fragment species and/or reference fragment species. In certain embodiments, nucleic acid is enriched for a specific nucleic acid fragment length or range of fragment lengths using one or more length-based separation methods described below. In certain embodiments, nucleic acid is enriched for fragments from a select genomic region (e.g., chromosome) using one or more sequence-based separation methods described herein and/or known in the art. In certain embodiments, nucleic acid is enriched for fragments from known or suspected A/B compartment transition regions, for example all or a subset of A/B compartment transition regions in a genome, or a set of A/B compartment transitions associated with disease states such as cancer. In certain embodiments, nucleic acid is enriched for fragments from known driver mutation regions. In certain embodiments, nucleic acid is enriched for fragments from regulatory regions.

Non-limiting examples of methods for enriching for a nucleic acid subpopulation in a sample include methods that exploit epigenetic differences between nucleic acid species (e.g., methylation-based fetal nucleic acid enrichment methods described in U.S. Patent Application Publication No. 2010/0105049, which is incorporated by reference herein); restriction endonuclease enhanced polymorphic sequence approaches (e.g., such as a method described in U.S. Patent Application Publication No. 2009/0317818, which is incorporated by reference herein); selective enzymatic degradation approaches; massively parallel signature sequencing (MPSS) approaches; amplification (e.g., PCR)-based approaches (e.g., loci-specific amplification methods, multiplex SNP allele PCR approaches; universal amplification methods); pull-down approaches (e.g., biotinylated ultramer pull-down methods); extension and ligation-based methods (e.g., molecular inversion probe (MIP) extension and ligation); and combinations thereof.

In some embodiments, modified nucleic acids can be enriched for. Nucleic acid modifications include but are not limited to carboxycytosine, 5-methylcytosine (5mC) and its oxidative derivatives (e.g., 5-hydroxymethylcytosine (5hmC), 5-formylcytosine (5f0), and 5-arboxylcytosine (5caC)), N(6)-methyladenine (6 mA), N4-methylcytosine (4mC), N(6)-methyladenosine (m(6)A), pseudouridine (ψ), 5-methylcytidine (m(5)C), hydroxymethyl uracil, 2′-O-methylation at the 3′ end, tRNA modifications, miRNA modifications, and snRNA modifications. Nucleic acids comprising one or more modifications can be enriched for by a variety of methods, including but not limited to antibody-based pulldown. Modified nucleic acid enrichment can be conducted before or after denaturation of dsDNA. Enrichment prior to denaturation can result in also enriching for the complementary strand which may lack the modification, while enrichment after denaturation does not enrich for complementary strands lacking modification.

In some embodiments, nucleic acid is enriched for fragments from a select genomic region (e.g., chromosome) using one or more sequence-based separation methods described herein. Sequence-based separation generally is based on nucleotide sequences present in the fragments of interest (e.g., target and/or reference fragments) and substantially not present in other fragments of the sample or present in an insubstantial amount of the other fragments (e.g., 5% or less). In some embodiments, sequence-based separation can generate separated target fragments and/or separated reference fragments. Separated target fragments and/or separated reference fragments often are isolated away from the remaining fragments in the nucleic acid sample. In certain embodiments, the separated target fragments and the separated reference fragments also are isolated away from each other (e.g., isolated in separate assay compartments). In certain embodiments, the separated target fragments and the separated reference fragments are isolated together (e.g., isolated in the same assay compartment). In some embodiments, unbound fragments can be differentially removed or degraded or digested.

In some embodiments, scaffold adapters are used to enrich for target nucleic acids. For example, scaffold adapters can be designed such that some or all of the bases in the ssNA hybridization region are defined or known bases. These scaffold adapters can hybridize preferentially to target nucleic acids with sequences complementary to the defined or known bases of the scaffold adapter ssNA hybridization region, thereby enriching for the target nucleic acids in the resulting library. For example, including a GC dinucleotide in the ssNA hybridization region can be used to enrich for target nucleic acids that have terminal CG (also called CpG) dinucleotides. Any other defined sequence can be targeted in a similar manner, using some or all of the length of the scaffold adapter ssNA hybridization region, including but not limited to nuclease cleavage sites, gene promoter regions, pathogen sequences, tumor-related sequences, and other motifs. In an example, libraries were prepared using non-enriching scaffold adapters and CG dinucleotide enriching scaffold adapters. For libraries prepared without enrichment, 1.7% of reads started with CG and 1.1% of reads ended with CG. For libraries prepared with enrichment, 5.2% of reads started with CG and 19.6% of reads ended with CG. In another example, a sample comprising RNA (e.g., host and pathogen RNA) is reverse transcribed with primers specific to pathogen RNA of interest to generate cDNA; the cDNA is then purified and prepared with single-stranded library preparation methods as discussed herein, either with standard scaffold adapters or with scaffold adapters with ssNA hybridization regions targeted to the regions enriched by the reverse transcription primers. Pathogenic DNA can be similarly enriched.

In some instances, the target nucleic acid sequence at the 5′ or 3′ nucleic acid termini is defined or known. In other instances, scaffold adapters can be used to identify novel targets of interest at 5′ or 3′ nucleic acid termini. Nucleic acid sequences or patterns of interest may be characterized from the scaffold adapter library output with or without enrichment. In some instances, a specific sequence or sequence pattern at 5′, 3′, or both nucleic acid termini may be associated with a particular state. Such states include but are not limited to disease state, methylation state, and gene expression state. The scaffold adapters can be used to quantify the presence or relative abundance of a known or novel target sequence(s) at nucleic acid termini between samples and controls, for example, cell-free DNA from cancer patients and healthy controls. These data can be used to learn the relationship between the sequence information at DNA termini and a given state. By training on a well-characterized dataset of patient and healthy samples, in one example, an analytical method or algorithm can be used to predict the state or transitions through the state. For example, the increase of AT dinucleotides and reduction of CpG dinucleotides was observed at 5′ and 3′ DNA termini in cfDNA from patients with Acute Myeloid leukemia (AML) when compared to non-AML patient samples. In this example, an analytical tool may be used for cfDNA termini sequence information to predict a person's risk for developing AML.

In some embodiments, a selective nucleic acid capture process is used to separate target and/or reference fragments away from a nucleic acid sample and/or enrich a nucleic acid sample for one or more genomic regions of interest. Commercially available nucleic acid capture systems include, for example, Nimblegen sequence capture system (Roche NimbleGen, Madison, WI); ILLUMINA BEADARRAY platform (Illumina, San Diego, CA); Affymetrix GENECHIP platform (Affymetrix, Santa Clara, CA); Agilent SureSelect Target Enrichment System (Agilent Technologies, Santa Clara, CA); and related platforms. Such methods typically involve hybridization of a capture oligonucleotide to a part or all of the nucleotide sequence of a target or reference fragment and can include use of a solid phase (e.g., solid phase array) and/or a solution based platform. Capture oligonucleotides (sometimes referred to as “bait”) can be selected or designed such that they preferentially hybridize to nucleic acid fragments from selected genomic regions or loci, or a particular sequence in a nucleic acid target. In certain embodiments, a hybridization-based method (e.g., using oligonucleotide arrays) can be used to enrich for fragments containing certain nucleic acid sequences. Thus, in some embodiments, a nucleic acid sample is optionally enriched by capturing a subset of fragments using capture oligonucleotides complementary to, for example, selected sequences in sample nucleic acid. In certain instances, captured fragments are amplified.

For example, captured fragments containing adapters may be amplified using primers complementary to the adapter sequences to form collections of amplified fragments, indexed according to adapter sequence. In some embodiments, nucleic acid is enriched for fragments from a select genomic region (e.g., chromosome, a gene) by amplification of one or more regions of interest using oligonucleotides (e.g., PCR primers) complementary to sequences in fragments containing the region(s) of interest, or part(s) thereof.

In some embodiments, nucleic acid is enriched for a particular nucleic acid fragment length, range of lengths, or lengths under or over a particular threshold or cutoff using one or more length-based separation methods. Nucleic acid fragment length typically refers to the number of nucleotides in the fragment. Nucleic acid fragment length also is sometimes referred to as nucleic acid fragment size. In some embodiments, a length-based separation method is performed without measuring lengths of individual fragments. In some embodiments, a length based separation method is performed in conjunction with a method for determining length of individual fragments. In some embodiments, length-based separation refers to a size fractionation procedure where all or part of the fractionated pool can be isolated (e.g., retained) and/or analyzed. Size fractionation procedures are known in the art (e.g., separation on an array, separation by a molecular sieve, separation by gel electrophoresis, separation by column chromatography (e.g., size-exclusion columns), and microfluidics-based approaches). In certain instances, length-based separation approaches can include selective sequence tagging approaches, fragment circularization, chemical treatment (e.g., formaldehyde, polyethylene glycol (PEG) precipitation), mass spectrometry and/or size-specific nucleic acid amplification, for example.

In some embodiments, nucleic acid is enriched for fragments associated with one or more nucleic acid binding proteins. Example enrichment methods include but are not limited to chromatin immunoprecipitation (ChIP), cross-linked ChIP (XCHIP), native ChIP (NChIP), bead-free ChIP, carrier ChIP (CChIP), fast ChIP (qChIP), quick and quantitative ChIP (Q²ChIP), microchip (pChIP), matrix ChIP, pathology-ChIP (PAT-ChIP), ChIP-exo, ChIP-on-chip, RIP-ChIP, HiChIP, ChIA-PET, and HiChIRP.

In some embodiments, a method herein includes enriching an RNA species in a mixture of RNA species. For example, a method herein may comprise enriching messenger RNA (mRNA) present in a mixture of mRNA and ribosomal RNA (rRNA). Any suitable mRNA enrichment method may be used, which includes rRNA depletion and/or mRNA enrichment methods such as rRNA depletion with magnetic beads (e.g., Ribo-zero™, Ribominus™, and MICROBExpress™, which use rRNA depletion probes in combination with magnetic beads to deplete rRNAs from a sample, thus enriching mRNAs), oligo(dT)-based poly(A) enrichment (e.g., BioMag® Oligo (dT)20), nuclease-based rRNA depletion (e.g., digestion of rRNA with Terminator™ 5′-Phosphate Dependent Exonuclease), and combinations thereof.

Enrichment strategies can increase the relative abundance (e.g., as assessed by percent of sequencing reads) of the targeted nucleic acids by at least 5%, 10%, 20%, 30%, 40%, 50%, 60%, 70%, 80%, 90%, 100%, 200%, 300%, 400%, 500%, 600%, 700%, 800%, 900%, 1000%, 1100%, 1200%, 1300%, 1400%, 1500%, 1600%, 1700%, 1800%, 1900%, 2000%, 3000%, 4000%, 5000%, 6000%, 7000%, 8000%, 9000%, 10000%, or more.

Length-Based Separation

In some embodiments, a method herein comprises separating target nucleic acids (e.g., ssNAs) according to fragment length. For example, target nucleic acids (e.g., ssNAs) may be enriched for a particular nucleic acid fragment length, range of lengths, or lengths under or over a particular threshold or cutoff using one or more length-based separation methods. Nucleic acid fragment length typically refers to the number of nucleotides in the fragment. Nucleic acid fragment length also may be referred to as nucleic acid fragment size. In some embodiments, a length-based separation method is performed without measuring lengths of individual fragments. In some embodiments, a length based separation method is performed in conjunction with a method for determining length of individual fragments. In some embodiments, length-based separation refers to a size fractionation procedure where all or part of the fractionated pool can be isolated (e.g., retained) and/or analyzed. Size fractionation procedures are known in the art (e.g., separation on an array, separation by a molecular sieve, separation by gel electrophoresis, separation by capillary electrophoresis, separation by column chromatography (e.g., size-exclusion columns), and microfluidics-based approaches). In some embodiments, length-based separation approaches can include fragment circularization, chemical treatment (e.g., formaldehyde, polyethylene glycol (PEG)), mass spectrometry and/or size-specific nucleic acid amplification, for example. In some embodiments, length based-separation is performed using Solid Phase Reversible Immobilization (SPRI) beads.

In some embodiments, nucleic acid fragments of a certain length, range of lengths, or lengths under or over a particular threshold or cutoff are separated from the sample. In some embodiments, fragments having a length under a particular threshold or cutoff (e.g., 500 bp, 400 bp, 300 bp, 200 bp, 150 bp, 100 bp) are referred to as “short” fragments and fragments having a length over a particular threshold or cutoff (e.g., 500 bp, 600 bp, 700 bp, 800 bp, 900 bp, 1000 bp) are referred to as “long” fragments, large fragments, and/or high molecular weight (HMW) fragments. In some embodiments, fragments of a certain length, range of lengths, or lengths under or over a particular threshold or cutoff are retained for analysis while fragments of a different length or range of lengths, or lengths over or under the threshold or cutoff are not retained for analysis. In some embodiments, fragments that are less than about 500 bp are retained. In some embodiments, fragments that are less than about 400 bp are retained. In some embodiments, fragments that are less than about 300 bp are retained. In some embodiments, fragments that are less than about 200 bp are retained. In some embodiments, fragments that are less than about 150 bp are retained. For example, fragments that are less than about 190 bp, 180 bp, 170 bp, 160 bp, 150 bp, 140 bp, 130 bp, 120 bp, 110 bp or 100 bp are retained. In some embodiments, fragments that are about 100 bp to about 200 bp are retained. For example, fragments that are about 190 bp, 180 bp, 170 bp, 160 bp, 150 bp, 140 bp, 130 bp, 120 bp or 110 bp are retained. In some embodiments, fragments that are in the range of about 100 bp to about 200 bp are retained. For example, fragments that are in the range of about 110 bp to about 190 bp, 130 bp to about 180 bp, 140 bp to about 170 bp, 140 bp to about 150 bp, 150 bp to about 160 bp, or 145 bp to about 155 bp are retained.

In some embodiments, target nucleic acids (e.g., ssNAs) having fragment lengths of less than about 1000 bp are combined with a plurality or pool of scaffold adapter species, or components of scaffold adapter species, described herein. In some embodiments, target nucleic acids (e.g., ssNAs) having fragment lengths of less than about 500 bp are combined with a plurality or pool of scaffold adapter species, or components of scaffold adapter species, described herein. In some embodiments, target nucleic acids (e.g., ssNAs) having fragment lengths of less than about 400 bp are combined with a plurality or pool of scaffold adapter species, or components of scaffold adapter species, described herein. In some embodiments, target nucleic acids (e.g., ssNAs) having fragment lengths of less than about 300 bp are combined with a plurality or pool of scaffold adapter species, or components of scaffold adapter species, described herein. In some embodiments, target nucleic acids (e.g., ssNAs) having fragment lengths of less than about 200 bp are combined with a plurality or pool of scaffold adapter species, or components of scaffold adapter species, described herein. In some embodiments, target nucleic acids (e.g., ssNAs) having fragment lengths of less than about 100 bp are combined with a plurality or pool of scaffold adapter species, or components of scaffold adapter species, described herein.

In some embodiments, target nucleic acids (e.g., ssNAs) having fragment lengths of about 100 bp or more are combined with a plurality or pool of scaffold adapter species, or components of scaffold adapter species, described herein. In some embodiments, target nucleic acids (e.g., ssNAs) having fragment lengths of about 200 bp or more are combined with a plurality or pool of scaffold adapter species, or components of scaffold adapter species, described herein. In some embodiments, target nucleic acids (e.g., ssNAs) having fragment lengths of about 300 bp or more are combined with a plurality or pool of scaffold adapter species, or components of scaffold adapter species, described herein. In some embodiments, target nucleic acids (e.g., ssNAs) having fragment lengths of about 400 bp or more are combined with a plurality or pool of scaffold adapter species, or components of scaffold adapter species, described herein. In some embodiments, target nucleic acids (e.g., ssNAs) having fragment lengths of about 500 bp or more are combined with a plurality or pool of scaffold adapter species, or components of scaffold adapter species, described herein. In some embodiments, target nucleic acids (e.g., ssNAs) having fragment lengths of about 1000 bp or more are combined with a plurality or pool of scaffold adapter species, or components of scaffold adapter species, described herein.

In some embodiments, target nucleic acids (e.g., ssNAs) having any fragment length or any combination of fragment lengths are combined with a plurality or pool of scaffold adapter species, or components of scaffold adapter species, described herein. For example, target nucleic acids (e.g., ssNAs) having fragment lengths of less than 500 bp and fragments lengths of 500 bp or more may be combined with a plurality or pool of scaffold adapter species, or components of scaffold adapter species, described herein.

Certain length-based separation methods that can be used with methods described herein employ a selective sequence tagging approach, for example. In such methods, a fragment size species (e.g., short fragments) nucleic acids are selectively tagged in a sample that includes long and short nucleic acids. Such methods typically involve performing a nucleic acid amplification reaction using a set of nested primers which include inner primers and outer primers. In some embodiments, one or both of the inner can be tagged to thereby introduce a tag onto the target amplification product. The outer primers generally do not anneal to the short fragments that carry the (inner) target sequence. The inner primers can anneal to the short fragments and generate an amplification product that carries a tag and the target sequence. Typically, tagging of the long fragments is inhibited through a combination of mechanisms which include, for example, blocked extension of the inner primers by the prior annealing and extension of the outer primers. Enrichment for tagged fragments can be accomplished by any of a variety of methods, including for example, exonuclease digestion of single stranded nucleic acid and amplification of the tagged fragments using amplification primers specific for at least one tag.

Another length-based separation method that can be used with methods described herein involves subjecting a nucleic acid sample to polyethylene glycol (PEG) precipitation. Examples of methods include those described in International Patent Application Publication Nos. WO2007/140417 and WO2010/115016. This method in general entails contacting a nucleic acid sample with PEG in the presence of one or more monovalent salts under conditions sufficient to substantially precipitate large nucleic acids without substantially precipitating small (e.g., less than 300 nucleotides) nucleic acids.

Another length-based enrichment method that can be used with methods described herein involves circularization by ligation, for example, using circligase. Short nucleic acid fragments typically can be circularized with higher efficiency than long fragments. Non-circularized sequences can be separated from circularized sequences, and the enriched short fragments can be used for further analysis.

Another length-based separation method that can be used with methods described herein involves electrophoresis. Any electrophoresis method known in the art, whereby nucleic acids are separated by size, can be used in conjunction with the methods provided herein, which include, but are not limited to, standard electrophoretic techniques and specialized electrophoretic techniques, such as, for example capillary electrophoresis. In some instances, electrophoresis may be used for detection and quantification of nucleic acid fragments. In certain instances, electrophoresis may be used for determining nucleic fragment lengths. In some embodiments, capillary electrophoresis may be used for determining nucleic fragment lengths.

In some embodiments, capillary electrophoresis is used to separate, quantify, and/or determine lengths of nucleic fragments (e.g., amplified nucleic acid fragments; enriched nucleic acid fragments). Capillary electrophoresis (CE) encompasses a family of related separation techniques that use narrow-bore fused-silica capillaries to separate a complex array of large and small molecules, such as, for example, nucleic acids of varying length. High electric field strengths can be used to separate nucleic acid molecules based on differences in charge, size and hydrophobicity. Sample introduction is accomplished by immersing the end of the capillary into a sample vial and applying pressure, vacuum or voltage. Depending on the types of capillary and electrolytes used, the technology of CE can be segmented into several separation techniques, any of which can be adapted to the methods provided herein. Examples of these are provided below.

Capillary Zone Electrophoresis (CZE), also known as free-solution CE (FSCE), is the simplest form of CE. The separation mechanism is based on differences in the charge-to-mass ratio of the analytes. Fundamental to CZE are homogeneity of the buffer solution and constant field strength throughout the length of the capillary. The separation relies principally on the pH controlled dissociation of acidic groups on the solute or the protonation of basic functions on the solute.

Capillary Gel Electrophoresis (CGE) is the adaptation of traditional gel electrophoresis into the capillary using polymers in solution to create a molecular sieve also known as replaceable physical gel. This allows analytes having similar charge-to-mass ratios to be resolved by size. This technique is commonly employed in SDS-Gel molecular weight analysis of proteins and the sizing of applications of DNA sequencing and genotyping.

Capillary Isoelectric Focusing (CIEF) allows amphoteric molecules, such as proteins, to be separated by electrophoresis in a pH gradient generated between the cathode and anode. A solute will migrate to a point where its net charge is zero. At the solutes isoelectric point (pI), migration stops and the sample is focused into a tight zone. In CIEF, once a solute has focused at its pI, the zone is mobilized past the detector by either pressure or chemical means. This technique is commonly employed in protein characterization as a mechanism to determine a protein's isoelectric point.

Isotachophoresis (ITP) is a focusing technique based on the migration of the sample components between leading and terminating electrolytes. Solutes having mobilities intermediate to those of the leading and terminating electrolytes stack into sharp, focused zones.

Electrokinetic Chromatography (EKC) is a family of electrophoresis techniques named after electrokinetic phenomena, which include electroosmosis, electrophoresis and chromatography. A key example of this is seen with cyclodextrin-mediated EKC. Here the differential interaction of enantiomers with the cyclodextrins allows for the separation of chiral compounds.

Micellar Electrokinetic Capillary Chromatography (MECC OR MEKC) is a mode of electrokinetic chromatography in which surfactants are added to the buffer solution at concentrations that form micelles. The separation principle of MEKC is based on a differential partition between the micelle and the solvent. This principle can be employed with charged or neutral solutes and may involve stationary or mobile micelles. MEKC has great utility in separating mixtures that contain both ionic and neutral species.

Micro Emulsion Electrokinetic Chromatography (MEEKC) is a CE technique in which solutes partition with moving oil droplets in buffer. The microemulsion droplets are usually formed by sonicating immicible heptane or octane with water. SDS is added at relatively high concentrations to stabilize the emulsion. This allows the separation of both aqueous and water-insoluble compounds.

Non-Aqueous Capillary Electrophoresis (NACE) involves the separation of analytes in a medium composed of organic solvents. The viscosity and dielectric constants of organic solvents affect both sample ion mobility and the level of electroosmotic flow. The use of non-aqueous medium allows additional selectivity options in methods development and is also valuable for the separation of water-insoluble compounds.

Capillary Electrochromatography (CEC) is a hybrid separation method that couples the high separation efficiency of CZE with HPLC and uses an electric field rather than hydraulic pressure to propel the mobile phase through a packed bed. Because there is minimal backpressure, it is possible to use small-diameter packings and achieve very high efficiencies. Its most useful application appears to be in the form of on-line analyte concentration that can be used to concentrate a given sample prior to separation by CZE.

Any device, instrument or machine capable of performing capillary electrophoresis can be used in conjunction with the methods provided herein. In general, a capillary electrophoresis system's main components are a sample vial, source and destination vials, a capillary, electrodes, a high-voltage power supply, a detector, and a data output and handling device. The source vial, destination vial and capillary are filled with an electrolyte such as an aqueous buffer solution. To introduce the sample, the capillary inlet is placed into a vial containing the sample and then returned to the source vial (sample is introduced into the capillary via capillary action, pressure, or siphoning). The migration of the analytes (i.e. nucleic acids) is then initiated by an electric field that is applied between the source and destination vials and is supplied to the electrodes by the high-voltage power supply. Ions, positive or negative, are pulled through the capillary in the same direction by electroosmotic flow. The analytes (i.e. nucleic acids) separate as they migrate due to their electrophoretic mobility and are detected near the outlet end of the capillary. The output of the detector is sent to a data output and handling device such as an integrator or computer. The data is then displayed as an electropherogram, which can report detector response as a function of time. Separated nucleic acids can appear as peaks with different migration times in an electropherogram.

Separation by capillary electrophoresis can be detected by several detection devices. The majority of commercial systems use UV or UV-Vis absorbance as their primary mode of detection. In these systems, a section of the capillary itself is used as the detection cell. The use of on-tube detection enables detection of separated analytes with no loss of resolution. In general, capillaries used in capillary electrophoresis can be coated with a polymer for increased stability. The portion of the capillary used for UV detection is often optically transparent. The path length of the detection cell in capillary electrophoresis (˜50 micrometers) is far less than that of a traditional UV cell (˜1 cm). According to the Beer-Lambert law, the sensitivity of the detector is proportional to the path length of the cell. To improve the sensitivity, the path length can be increased, though this can result in a loss of resolution. The capillary tube itself can be expanded at the detection point, creating a “bubble cell” with a longer path length or additional tubing can be added at the detection point. Both of these methods, however, may decrease the resolution of the separation.

Fluorescence detection can also be used in capillary electrophoresis for samples that naturally fluoresce or are chemically modified to contain fluorescent tags, such as, for example, labeled nucleic acid fragments. This mode of detection offers high sensitivity and improved selectivity for these samples. The method requires that the light beam be focused on the capillary. Laser-induced fluorescence can be been used in CE systems with detection limits as low as 10-18 to 10-21 mol. The sensitivity of the technique is attributed to the high intensity of the incident light and the ability to accurately focus the light on the capillary.

Several capillary electrophoresis machines are known in the art and can be used in conjunction with the methods provided herein. These include, but are not limited to, CALIPER LAB CHIP GX (Caliper Life Sciences, Mountain View, CA), P/ACE 2000 Series (Beckman Coulter, Brea, CA), HP G1600A CE (Hewlett-Packard, Palo Alto, CA), AGILENT 7100 CE (Agilent Technologies, Santa Clara, CA), and ABI PRISM Genetic Analyzer (Applied Biosystems, Carlsbad, CA).

Nucleic Acid Library

Methods herein may include preparing a nucleic acid library and/or modifying nucleic acids for a nucleic acid library. In some embodiments, ends of nucleic acid fragments are modified such that the fragments, or amplified products thereof, may be incorporated into a nucleic acid library. Generally, a nucleic acid library refers to a plurality of polynucleotide molecules (e.g., a sample of nucleic acids) that are prepared, assembled and/or modified for a specific process, non-limiting examples of which include immobilization on a solid phase (e.g., a solid support, a flow cell, a bead), enrichment, amplification, cloning, detection and/or for nucleic acid sequencing. In certain embodiments, a nucleic acid library is prepared prior to or during a sequencing process. A nucleic acid library (e.g., sequencing library) can be prepared by a suitable method as known in the art. A nucleic acid library can be prepared by a targeted or a non-targeted preparation process.

In some embodiments, a library of nucleic acids is modified to comprise a chemical moiety (e.g., a functional group) configured for immobilization of nucleic acids to a solid support. In some embodiments a library of nucleic acids is modified to comprise a biomolecule (e.g., a functional group) and/or member of a binding pair configured for immobilization of the library to a solid support, non-limiting examples of which include thyroxin-binding globulin, steroid-binding proteins, antibodies, antigens, haptens, enzymes, lectins, nucleic acids, repressors, protein A, protein G, avidin, streptavidin, biotin, complement component C1q, nucleic acid-binding proteins, receptors, carbohydrates, oligonucleotides, polynucleotides, complementary nucleic acid sequences, the like and combinations thereof. Some examples of specific binding pairs include, without limitation: an avidin moiety and a biotin moiety; an antigenic epitope and an antibody or immunologically reactive fragment thereof; an antibody and a hapten; a digoxigenin moiety and an anti-digoxigenin antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; an oligonucleotide or polynucleotide and its corresponding complement; the like or combinations thereof.

In some embodiments, a library of nucleic acids is modified to comprise one or more polynucleotides of known composition, non-limiting examples of which include an identifier (e.g., a tag, an indexing tag), a capture sequence, a label, an adapter, a restriction enzyme site, a promoter, an enhancer, an origin of replication, a stem loop, a complimentary sequence (e.g., a primer binding site, an annealing site), a suitable integration site (e.g., a transposon, a viral integration site), a modified nucleotide, a unique molecular identifier (UMI) described herein, a palindromic sequence described herein, the like or combinations thereof. Polynucleotides of known sequence can be added at a suitable position, for example on the 5′ end, 3′ end or within a nucleic acid sequence. Polynucleotides of known sequence can be the same or different sequences. In some embodiments, a polynucleotide of known sequence is configured to hybridize to one or more oligonucleotides immobilized on a surface (e.g., a surface in flow cell). For example, a nucleic acid molecule comprising a 5′ known sequence may hybridize to a first plurality of oligonucleotides while the 3′ known sequence may hybridize to a second plurality of oligonucleotides. In some embodiments, a library of nucleic acid can comprise chromosome-specific tags, capture sequences, labels and/or adapters (e.g., oligonucleotide adapters described herein). In some embodiments, a library of nucleic acids comprises one or more detectable labels. In some embodiments one or more detectable labels may be incorporated into a nucleic acid library at a 5′ end, at a 3′ end, and/or at any nucleotide position within a nucleic acid in the library. In some embodiments, a library of nucleic acids comprises hybridized oligonucleotides. In certain embodiments hybridized oligonucleotides are labeled probes. In some embodiments, a library of nucleic acids comprises hybridized oligonucleotide probes prior to immobilization on a solid phase.

In some embodiments, a polynucleotide of known sequence comprises a universal sequence. A universal sequence is a specific nucleotide sequence that is integrated into two or more nucleic acid molecules or two or more subsets of nucleic acid molecules where the universal sequence is the same for all molecules or subsets of molecules that it is integrated into. A universal sequence is often designed to hybridize to and/or amplify a plurality of different sequences using a single universal primer that is complementary to a universal sequence. In some embodiments two (e.g., a pair) or more universal sequences and/or universal primers are used. A universal primer often comprises a universal sequence. In some embodiments adapters (e.g., universal adapters) comprise universal sequences. In some embodiments one or more universal sequences are used to capture, identify and/or detect multiple species or subsets of nucleic acids.

In certain embodiments of preparing a nucleic acid library, (e.g., in certain sequencing by synthesis procedures), nucleic acids are size selected and/or fragmented into lengths of several hundred base pairs, or less (e.g., in preparation for library generation). In some embodiments, library preparation is performed without fragmentation (e.g., when using cell-free DNA).

In certain embodiments, a ligation-based library preparation method is used (e.g., ILLUMINA TRUSEQ, Illumina, San Diego CA). Ligation-based library preparation methods often make use of an adapter (e.g., a methylated adapter) design which can incorporate an index sequence (e.g., a sample index sequence to identify sample origin for a nucleic acid sequence) at the initial ligation step and often can be used to prepare samples for single-read sequencing, paired-end sequencing and multiplexed sequencing. For example, nucleic acids (e.g., fragmented nucleic acids or cell-free DNA) may be end repaired by a fill-in reaction, an exonuclease reaction or a combination thereof. In some embodiments, the resulting blunt-end repaired nucleic acid can then be extended by a single nucleotide, which is complementary to a single nucleotide overhang on the 3′ end of an adapter/primer. Any nucleotide can be used for the extension/overhang nucleotides. In some embodiments, end repair is omitted and scaffold adapters (e.g., scaffold adapters described herein) are ligated directly to the native ends of nucleic acids (e.g., single-stranded nucleic acids, fragmented nucleic acids, and/or cell-free DNA).

In some embodiments, nucleic acid library preparation comprises ligating a scaffold adapter, or component thereof, (e.g., to a sample nucleic acid, to a sample nucleic acid fragment, to a template nucleic acid, to a target nucleic acid, to an ssNA), such as a scaffold adapter described herein. Scaffold adapters, or components thereof, may comprise sequences complementary to flow-cell anchors, and sometimes are utilized to immobilize a nucleic acid library to a solid support, such as the inside surface of a flow cell, for example. In some embodiments, a scaffold adapter, or component thereof, comprises an identifier, one or more sequencing primer hybridization sites (e.g., sequences complementary to universal sequencing primers, single end sequencing primers, paired end sequencing primers, multiplexed sequencing primers, and the like), or combinations thereof (e.g., adapter/sequencing, adapter/identifier, adapter/identifier/sequencing). In some embodiments, a scaffold adapter, or component thereof, comprises one or more of primer annealing polynucleotide, also referred to herein as priming sequence or primer binding domain, (e.g., for annealing to flow cell attached oligonucleotides and/or to free amplification primers), an index polynucleotide (e.g., sample index sequence for tracking nucleic acid from different samples; also referred to as a sample ID), a barcode polynucleotide (e.g., single molecule barcode (SMB) for tracking individual molecules of sample nucleic acid that are amplified prior to sequencing; also referred to as a molecular barcode or a unique molecular identifier (UMI)). In some embodiments, a primer annealing component (or priming sequence or primer binding domain) of a scaffold adapter, or component thereof, comprises one or more universal sequences (e.g., sequences complementary to one or more universal amplification primers). In some embodiments, an index polynucleotide (e.g., sample index; sample ID) is a component of a scaffold adapter, or component thereof. In some embodiments, an index polynucleotide (e.g., sample index; sample ID) is a component of a universal amplification primer sequence.

In some embodiments, scaffold adapters, or components thereof, when used in combination with amplification primers (e.g., universal amplification primers) are designed generate library constructs comprising one or more of: universal sequences, molecular barcodes (UMIs), UMI flanking sequence, sample ID sequences, spacer sequences, and a sample nucleic acid sequence (e.g., ssNA sequence). In some embodiments, scaffold adapters, or components thereof, when used in combination with universal amplification primers are designed to generate library constructs comprising an ordered combination of one or more of: universal sequences, molecular barcodes (UMIs), sample ID sequences, spacer sequences, and a sample nucleic acid sequence (e.g., ssNA sequence). For example, a library construct may comprise a first universal sequence, followed by a second universal sequence, followed by first molecular barcode (UMI), followed by a spacer sequence, followed by a template sequence (e.g., sample nucleic acid sequence; ssNA sequence), followed by a spacer sequence, followed by a second molecular barcode (UMI), followed by a third universal sequence, followed by a sample ID, followed by a fourth universal sequence. In some embodiments, scaffold adapters, or components thereof, when used in combination with amplification primers (e.g., universal amplification primers) are designed generate library constructs for each strand of a template molecule (e.g., sample nucleic acid molecule; ssNA molecule). In some embodiments, scaffold adapters are duplex adapters.

An identifier can be a suitable detectable label incorporated into or attached to a nucleic acid (e.g., a polynucleotide) that allows detection and/or identification of nucleic acids that comprise the identifier. In some embodiments, an identifier is incorporated into or attached to a nucleic acid during a sequencing method (e.g., by a polymerase). In some embodiments, an identifier is incorporated into or attached to a nucleic acid prior to a sequencing method (e.g., by an extension reaction, by an amplification reaction, by a ligation reaction). Non-limiting examples of identifiers include nucleic acid tags, nucleic acid indexes or barcodes, a radiolabel (e.g., an isotope), metallic label, a fluorescent label, a chemiluminescent label, a phosphorescent label, a fluorophore quencher, a dye, a protein (e.g., an enzyme, an antibody or part thereof, a linker, a member of a binding pair), the like or combinations thereof. In some embodiments, an identifier (e.g., a nucleic acid index or barcode) is a unique, known and/or identifiable sequence of nucleotides or nucleotide analogues. In some embodiments, identifiers are six or more contiguous nucleotides. A multitude of fluorophores are available with a variety of different excitation and emission spectra. Any suitable type and/or number of fluorophores can be used as an identifier. In some embodiments 1 or more, 2 or more, 3 or more, 4 or more, 5 or more, 6 or more, 7 or more, 8 or more, 9 or more, 10 or more, 20 or more, 30 or more or 50 or more different identifiers are utilized in a method described herein (e.g., a nucleic acid detection and/or sequencing method). In some embodiments, one or two types of identifiers (e.g., fluorescent labels) are linked to each nucleic acid in a library. Detection and/or quantification of an identifier can be performed by a suitable method, apparatus or machine, non-limiting examples of which include flow cytometry, quantitative polymerase chain reaction (qPCR), gel electrophoresis, a luminometer, a fluorometer, a spectrophotometer, a suitable gene-chip or microarray analysis, Western blot, mass spectrometry, chromatography, cytofluorimetric analysis, fluorescence microscopy, a suitable fluorescence or digital imaging method, confocal laser scanning microscopy, laser scanning cytometry, affinity chromatography, manual batch mode separation, electric field suspension, a suitable nucleic acid sequencing method and/or nucleic acid sequencing apparatus, the like and combinations thereof.

In some embodiments, an identifier, a sequencing-specific index/barcode, and a sequencer-specific flow-cell binding primer sites are incorporated into a nucleic acid library by single-primer extension (e.g., by a strand displacing polymerase).

In some embodiments, a nucleic acid library or parts thereof are amplified (e.g., amplified by a PCR-based method) under amplification conditions. In some embodiments, a sequencing method comprises amplification of a nucleic acid library. A nucleic acid library can be amplified prior to or after immobilization on a solid support (e.g., a solid support in a flow cell). Nucleic acid amplification includes the process of amplifying or increasing the numbers of a nucleic acid template and/or of a complement thereof that are present (e.g., in a nucleic acid library), by producing one or more copies of the template and/or its complement. Amplification can be carried out by a suitable method. A nucleic acid library can be amplified by a thermocycling method or by an isothermal amplification method. In some embodiments, a rolling circle amplification method is used. In some embodiments, amplification takes place on a solid support (e.g., within a flow cell) where a nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support. In some embodiments, modified nucleic acid (e.g., nucleic acid modified by addition of adapters) is amplified.

In some embodiments, solid phase amplification comprises a nucleic acid amplification reaction comprising only one species of oligonucleotide primer immobilized to a surface. In certain embodiments, solid phase amplification comprises a plurality of different immobilized oligonucleotide primer species. In some embodiments, solid phase amplification may comprise a nucleic acid amplification reaction comprising one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WildFire amplification (e.g., U.S. Patent Application Publication No. 2013/0012399), the like or combinations thereof.

Nucleic Acid Sequencing

In some embodiments, nucleic acid (e.g., nucleic acid fragments, sample nucleic acid, cell-free nucleic acid, single-stranded nucleic acid, single-stranded DNA, single-stranded RNA) is sequenced. In some embodiments, ssNA hybridized to scaffold adapters provided herein (“hybridization products”) are sequenced by a sequencing process. In some embodiments, ssNA ligated to oligonucleotide components provided herein (“single-stranded ligation products”) are sequenced by a sequencing process. In some embodiments, hybridization products and/or single-stranded ligation products are amplified by an amplification process, and the amplification products are sequenced by a sequencing process. In some embodiments, hybridization products and/or single-stranded ligation products are not amplified by an amplification process, and the hybridization products and/or single-stranded ligation products are sequenced without prior amplification by a sequencing process. In some embodiments, the sequencing process generates sequence reads (or sequencing reads). In some embodiments, a method herein comprises determining the sequence of a single-stranded nucleic acid molecule based on the sequence reads.

In some embodiments, a sequencing process herein is a whole genome sequencing process. In some embodiments, a sequencing process herein is a genome-wide sequencing process. In some embodiments, a sequencing process herein comprises massively parallel sequencing (i.e., nucleic acid molecules are sequenced in a massively parallel fashion, typically within a flow cell). In some embodiments, a sequencing process herein is a shotgun sequencing process. In some embodiments, a sequencing process herein is a non-locus-specific sequencing process. In some embodiments, a sequencing process herein is a non-targeted sequencing process. In some embodiments, a sequencing process herein comprises single-end sequencing. In some embodiments, nucleic acid fragment lengths are determined according to the length of a single-end sequencing read. In some embodiments, a sequencing process herein comprises paired-end sequencing. In some embodiments, nucleic acid fragment lengths are determined according to mapped positions of paired-end sequencing reads.

For certain sequencing platforms (e.g., paired-end sequencing), generating sequence reads may include generating forward sequence reads and generating reverse sequence reads. For example, sequencing using certain paired-end sequencing platforms sequence each nucleic acid fragment from both directions, generally resulting in two reads per nucleic acid fragment, with the first read in a forward orientation (forward read) and the second read in reverse-complement orientation (reverse read). For certain platforms, a forward read is generated off a particular primer within a sequencing adapter (e.g., ILLUMINA adapter, P5 primer), and a reverse read is generated off a different primer within a sequencing adapter (e.g., ILLUMINA adapter, P7 primer).

Nucleic acid may be sequenced using any suitable sequencing platform including a Sanger sequencing platform, a high throughput or massively parallel sequencing (next generation sequencing (NGS)) platform, or the like, such as, for example, a sequencing platform provided by Illumina® (e.g., HiSeq™, MiSeq™ and/or Genome Analyzer™ sequencing systems); Oxford Nanopore™ Technologies (e.g., MinION sequencing system), Ion Torrent™ (e.g., Ion PGM™ and/or Ion Proton™ sequencing systems); Pacific Biosciences (e.g., PACBIO RS II sequencing system); Life Technologies™ (e.g., SOLiD sequencing system); Roche (e.g., 454 GS FLX+ and/or GS Junior sequencing systems); or any other suitable sequencing platform. In some embodiments, the sequencing process is a highly multiplexed sequencing process. In certain instances, a full or substantially full sequence is obtained and sometimes a partial sequence is obtained. Nucleic acid sequencing generally produces a collection of sequence reads. As used herein, “reads” (e.g., “a read,” “a sequence read”) are short sequences of nucleotides produced by any sequencing process described herein or known in the art. Reads can be generated from one end of nucleic acid fragments (single-end reads), and sometimes are generated from both ends of nucleic acid fragments (e.g., paired-end reads, double-end reads). In some embodiments, a sequencing process generates short sequencing reads or “short reads.” In some embodiments, the nominal, average, mean or absolute length of short reads sometimes is about 10 continuous nucleotides to about 250 or more contiguous nucleotides. In some embodiments, the nominal, average, mean or absolute length of short reads sometimes is about 50 continuous nucleotides to about 150 or more contiguous nucleotides.

The length of a sequence read is often associated with the particular sequencing technology utilized. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, sequence reads are of a mean, median, average or absolute length of about 15 bp to about 900 bp long. In certain embodiments sequence reads are of a mean, median, average or absolute length of about 1000 bp or more. In some embodiments sequence reads are of a mean, median, average or absolute length of about 1500, 2000, 2500, 3000, 3500, 4000, 4500, or 5000 bp or more. In some embodiments, sequence reads are of a mean, median, average or absolute length of about 100 bp to about 200 bp.

In some embodiments. the nominal, average, mean or absolute length of single-end reads sometimes is about 10 continuous nucleotides to about 250 or more contiguous nucleotides, about 15 contiguous nucleotides to about 200 or more contiguous nucleotides, about 15 contiguous nucleotides to about 150 or more contiguous nucleotides, about 15 contiguous nucleotides to about 125 or more contiguous nucleotides, about 15 contiguous nucleotides to about 100 or more contiguous nucleotides, about 15 contiguous nucleotides to about 75 or more contiguous nucleotides, about 15 contiguous nucleotides to about 60 or more contiguous nucleotides, 15 contiguous nucleotides to about 50 or more contiguous nucleotides, about 15 contiguous nucleotides to about 40 or more contiguous nucleotides, and sometimes about 15 contiguous nucleotides or about 36 or more contiguous nucleotides. In certain embodiments the nominal, average, mean or absolute length of single-end reads is about 20 to about 30 bases, or about 24 to about 28 bases in length. In certain embodiments the nominal, average, mean or absolute length of single-end reads is about 1, 2, 3, 4, 5, 6, 7, 8, 9, 10, 11, 12, 13, 14, 15, 16, 17, 18, 19, 21, 22, 23, 24, 25, 26, 27, 28 or about 29 bases or more in length. In certain embodiments the nominal, average, mean or absolute length of single-end reads is about 20 to about 200 bases, about 100 to about 200 bases, or about 140 to about 160 bases in length. In certain embodiments the nominal, average, mean or absolute length of single-end reads is about 30, 40, 50, 60, 70, 80, 90, 100, 110, 120, 130, 140, 150, 160, 170, 180, 190, or about 200 bases or more in length. In certain embodiments, the nominal, average, mean or absolute length of paired-end reads sometimes is about 10 contiguous nucleotides to about 25 contiguous nucleotides or more (e.g., about 10, 11, 12, 13, 14, 15, 16, 17, 18, 19, 20, 21, 22, 23, 24 or 25 nucleotides in length or more), about 15 contiguous nucleotides to about 20 contiguous nucleotides or more, and sometimes is about 17 contiguous nucleotides or about 18 contiguous nucleotides. In certain embodiments, the nominal, average, mean or absolute length of paired-end reads sometimes is about 25 contiguous nucleotides to about 400 contiguous nucleotides or more (e.g., about 25, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 nucleotides in length or more), about 50 contiguous nucleotides to about 350 contiguous nucleotides or more, about 100 contiguous nucleotides to about 325 contiguous nucleotides, about 150 contiguous nucleotides to about 325 contiguous nucleotides, about 200 contiguous nucleotides to about 325 contiguous nucleotides, about 275 contiguous nucleotides to about 310 contiguous nucleotides, about 100 contiguous nucleotides to about 200 contiguous nucleotides, about 100 contiguous nucleotides to about 175 contiguous nucleotides, about 125 contiguous nucleotides to about 175 contiguous nucleotides, and sometimes is about 140 contiguous nucleotides to about 160 contiguous nucleotides. In certain embodiments, the nominal, average, mean, or absolute length of paired-end reads is about 150 contiguous nucleotides, and sometimes is 150 contiguous nucleotides.

Reads generally are representations of nucleotide sequences in a physical nucleic acid. For example, in a read containing an ATGC depiction of a sequence, “A” represents an adenine nucleotide, “T” represents a thymine nucleotide, “G” represents a guanine nucleotide and “C” represents a cytosine nucleotide, in a physical nucleic acid. Sequence reads obtained from a sample from a subject can be reads from a mixture of a minority nucleic acid and a majority nucleic acid. For example, sequence reads obtained from the blood of a cancer patient can be reads from a mixture of cancer nucleic acid and non-cancer nucleic acid. In another example, sequence reads obtained from the blood of a pregnant female can be reads from a mixture of fetal nucleic acid and maternal nucleic acid. In another example, sequence reads obtained from the blood of a patient having an infection or infectious disease can be reads from a mixture of host nucleic acid and pathogen nucleic acid. In another example, sequence reads obtained from the blood of a transplant recipient can be reads from a mixture of host nucleic acid and transplant nucleic acid. In another example, sequence reads obtained from a sample can be reads from a mixture of nucleic acid from microorganisms collectively comprising a microbiome (e.g., microbiome of gut, microbiome of blood, microbiome of mouth, microbiome of spinal fluid, microbiome of feces) in a subject. In another example, sequence reads obtained from a sample can be reads from a mixture of nucleic acid from microorganisms collectively comprising a microbiome (e.g., microbiome of gut, microbiome of blood, microbiome of mouth, microbiome of spinal fluid, microbiome of feces), and nucleic acid from the host subject. A mixture of relatively short reads can be transformed by processes described herein into a representation of genomic nucleic acid present in the subject, and/or a representation of genomic nucleic acid present in a tumor, a fetus, a pathogen, a transplant, or a microbiome.

In certain embodiments, “obtaining” nucleic acid sequence reads of a sample from a subject and/or “obtaining” nucleic acid sequence reads of a biological specimen from one or more reference persons can involve directly sequencing nucleic acid to obtain the sequence information. In some embodiments, “obtaining” can involve receiving sequence information obtained directly from a nucleic acid by another.

In some embodiments, some or all nucleic acids in a sample are enriched and/or amplified (e.g., non-specifically, e.g., by a PCR based method) prior to or during sequencing. In certain embodiments, specific nucleic acid species or subsets in a sample are enriched and/or amplified prior to or during sequencing. In some embodiments, a species or subset of a pre-selected pool of nucleic acids is sequenced randomly. In some embodiments, nucleic acids in a sample are not enriched and/or amplified prior to or during sequencing.

In some embodiments, a sequencing process generates a plurality of sequence reads. The plurality of sequence reads may be further processed (e.g., mapped, quantified, normalized) as described herein. In some embodiments, hundreds, thousands, tens of thousands, hundreds of thousands, millions, tens of millions, hundreds of millions, or billions of sequence reads are generated by a sequencing process described herein. In some embodiments, a sequencing process generates thousands of sequence reads. In some embodiments, a sequencing process generates millions of sequence reads. In some embodiments, a sequencing process generates thousands to millions of sequence reads. In some embodiments, a sequencing process generates between about 100,000 reads to about 1 billion reads. In some embodiments, a sequencing process generates between about 500,000 reads to about 100 million reads. In some embodiments, a sequencing process generates between about 1 million reads to about 10 million reads. For example, a sequencing process may generate about 1 million reads, about 2 million reads, about 3 million reads, about 4 million reads, about 5 million reads, about 6 million reads, about 7 million reads, about 8 million reads, about 9 million reads, about 10 million reads. In some embodiments, a sequencing process generates about 100,000 or more reads. In some embodiments, a sequencing process generates about 500,000 or more reads. In some embodiments, a sequencing process generates about 1 million or more reads. In some embodiments, a sequencing process generates about 5 million or more reads. In some embodiments, a sequencing process generates about 10 million or more reads.

In some embodiments, a representative fraction of a genome is sequenced and is sometimes referred to as “coverage” or “fold coverage.” For example, a 1-fold coverage indicates that roughly 100% of the nucleotide sequences of the genome are represented by reads. In some instances, fold coverage is referred to as (and is directly proportional to) “sequencing depth.” In some embodiments, “fold coverage” is a relative term referring to a prior sequencing run as a reference. For example, a second sequencing run may have 2-fold less coverage than a first sequencing run. In some embodiments, a genome is sequenced with redundancy, where a given region of the genome can be covered by two or more reads or overlapping reads (e.g., a “fold coverage” greater than 1, e.g., a 2-fold coverage). In some embodiments, a genome (e.g., a whole genome) is sequenced with about 0.01-fold to about 100-fold coverage, about 0.1-fold to 20-fold coverage, or about 0.1-fold to about 1-fold coverage (e.g., about 0.015-, 0.02-, 0.03-, 0.04-, 0.05-, 0.06-, 0.07-, 0.08-, 0.09-, 0.1-, 0.2-, 0.3-, 0.4-, 0.5-, 0.6-, 0.7-, 0.8-, 0.9-, 1-, 2-, 3-, 4-, 5-, 6-, 7-, 8-, 9-, 10-, 15-, 20-, 30-, 40-, 50-, 60-, 70-, 80-, 90-fold or greater coverage). In some embodiments, a sequencing process is performed at about 0.01-fold coverage to about 1-fold coverage. In some embodiments, a sequencing process is performed at about 0.02-fold coverage. In some embodiments, a sequencing process is performed at about 0.05-fold coverage. In some embodiments, a sequencing process is performed at about 0.1-fold coverage. In some embodiments, a sequencing process is performed at about 1-fold coverage to about 30-fold coverage. In some embodiments, a sequencing process is performed at about 5-fold coverage. In some embodiments, a sequencing process is performed at a coverage of at least about 0.01-fold. In some embodiments, a sequencing process is performed at a coverage of at least about 0.1-fold. In some embodiments, a sequencing process is performed at a coverage of at least about 1-fold. In some embodiments, a sequencing process is performed at a coverage of about 0.01-fold or less. In some embodiments, a sequencing process is performed at a coverage of about 0.1-fold or less. In some embodiments, a sequencing process is performed at a coverage of about 1-fold or less.

In some embodiments, specific parts of a genome (e.g., genomic parts from targeted methods) are sequenced and fold coverage values generally refer to the fraction of the specific genomic parts sequenced (i.e., fold coverage values do not refer to the whole genome). In some instances, specific genomic parts are sequenced at 1000-fold coverage or more. For example, specific genomic parts may be sequenced at 2000-fold, 5,000-fold, 10,000-fold, 20,000-fold, 30,000-fold, 40,000-fold or 50,000-fold coverage. In some embodiments, sequencing is at about 1,000-fold to about 100,000-fold coverage. In some embodiments, sequencing is at about 10,000-fold to about 70,000-fold coverage. In some embodiments, sequencing is at about 20,000-fold to about 60,000-fold coverage. In some embodiments, sequencing is at about 30,000-fold to about 50,000-fold coverage.

In some embodiments, one nucleic acid sample from one individual is sequenced. In certain embodiments, nucleic acids from each of two or more samples are sequenced, where samples are from one individual or from different individuals. In certain embodiments, nucleic acid samples from two or more biological samples are pooled, where each biological sample is from one individual or two or more individuals, and the pool is sequenced. In the latter embodiments, a nucleic acid sample from each biological sample often is identified by one or more unique identifiers.

In some embodiments, a sequencing method utilizes identifiers that allow multiplexing of sequence reactions in a sequencing process. The greater the number of unique identifiers, the greater the number of samples and/or chromosomes for detection, for example, that can be multiplexed in a sequencing process. A sequencing process can be performed using any suitable number of unique identifiers (e.g., 4, 8, 12, 24, 48, 96, or more).

A sequencing process sometimes makes use of a solid phase, and sometimes the solid phase comprises a flow cell on which nucleic acid from a library can be attached and reagents can be flowed and contacted with the attached nucleic acid. A flow cell sometimes includes flow cell lanes, and use of identifiers can facilitate analyzing a number of samples in each lane. A flow cell often is a solid support that can be configured to retain and/or allow the orderly passage of reagent solutions over bound analytes. Flow cells frequently are planar in shape, optically transparent, generally in the millimeter or sub-millimeter scale, and often have channels or lanes in which the analyte/reagent interaction occurs. In some embodiments, the number of samples analyzed in a given flow cell lane is dependent on the number of unique identifiers utilized during library preparation and/or probe design. Multiplexing using 12 identifiers, for example, allows simultaneous analysis of 96 samples (e.g., equal to the number of wells in a 96 well microwell plate) in an 8-lane flow cell. Similarly, multiplexing using 48 identifiers, for example, allows simultaneous analysis of 384 samples (e.g., equal to the number of wells in a 384 well microwell plate) in an 8-lane flow cell. Non-limiting examples of commercially available multiplex sequencing kits include Illumina's multiplexing sample preparation oligonucleotide kit and multiplexing sequencing primers and PhiX control kit (e.g., Illumina's catalog numbers PE-400-1001 and PE-400-1002, respectively).

Any suitable method of sequencing nucleic acids can be used, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first-generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used. In some embodiments, a high-throughput sequencing method is used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). In some embodiments, MPS sequencing methods utilize a targeted approach, where specific chromosomes, genes or regions of interest are sequenced. In certain embodiments, a non-targeted approach is used where most or all nucleic acids in a sample are sequenced, amplified and/or captured randomly.

In some embodiments a targeted enrichment, amplification and/or sequencing approach is used. A targeted approach often isolates, selects and/or enriches a subset of nucleic acids in a sample for further processing by use of sequence-specific oligonucleotides. In some embodiments, a library of sequence-specific oligonucleotides are utilized to target (e.g., hybridize to) one or more sets of nucleic acids in a sample. Sequence-specific oligonucleotides and/or primers are often selective for particular sequences (e.g., unique nucleic acid sequences) present in one or more chromosomes, genes, exons, introns, and/or regulatory regions of interest. Any suitable method or combination of methods can be used for enrichment, amplification and/or sequencing of one or more subsets of targeted nucleic acids. In some embodiments targeted sequences are isolated and/or enriched by capture to a solid phase (e.g., a flow cell, a bead) using one or more sequence-specific anchors. In some embodiments targeted sequences are enriched and/or amplified by a polymerase-based method (e.g., a PCR-based method, by any suitable polymerase-based extension) using sequence-specific primers and/or primer sets. Sequence specific anchors often can be used as sequence-specific primers.

MPS sequencing sometimes makes use of sequencing by synthesis and certain imaging processes. A nucleic acid sequencing technology that may be used in a method described herein is sequencing-by-synthesis and reversible terminator-based sequencing (e.g., Illumina's Genome Analyzer; Genome Analyzer II; HISEQ 2000; HISEQ 2500 (Illumina, San Diego CA)). With this technology, millions of nucleic acid (e.g., DNA) fragments can be sequenced in parallel. In one example of this type of sequencing technology, a flow cell is used which contains an optically transparent slide with 8 individual lanes on the surfaces of which are bound oligonucleotide anchors (e.g., adapter primers).

Sequencing by synthesis generally is performed by iteratively adding (e.g., by covalent addition) a nucleotide to a primer or preexisting nucleic acid strand in a template directed manner. Each iterative addition of a nucleotide is detected and the process is repeated multiple times until a sequence of a nucleic acid strand is obtained. The length of a sequence obtained depends, in part, on the number of addition and detection steps that are performed. In some embodiments of sequencing by synthesis, one, two, three or more nucleotides of the same type (e.g., A, G, C or T) are added and detected in a round of nucleotide addition. Nucleotides can be added by any suitable method (e.g., enzymatically or chemically). For example, in some embodiments a polymerase or a ligase adds a nucleotide to a primer or to a preexisting nucleic acid strand in a template directed manner. In some embodiments of sequencing by synthesis, different types of nucleotides, nucleotide analogues and/or identifiers are used. In some embodiments, reversible terminators and/or removable (e.g., cleavable) identifiers are used. In some embodiments, fluorescent labeled nucleotides and/or nucleotide analogues are used. In certain embodiments sequencing by synthesis comprises a cleavage (e.g., cleavage and removal of an identifier) and/or a washing step. In some embodiments the addition of one or more nucleotides is detected by a suitable method described herein or known in the art, non-limiting examples of which include any suitable imaging apparatus, a suitable camera, a digital camera, a CCD (Charge Couple Device) based imaging apparatus (e.g., a CCD camera), a CMOS (Complementary Metal Oxide Silicon) based imaging apparatus (e.g., a CMOS camera), a photo diode (e.g., a photomultiplier tube), electron microscopy, a field-effect transistor (e.g., a DNA field-effect transistor), an ISFET ion sensor (e.g., a CHEMFET sensor), the like or combinations thereof.

Any suitable MPS method, system or technology platform for conducting methods described herein can be used to obtain nucleic acid sequence reads. Non-limiting examples of MPS platforms include ILLUMINA/SOLEX/HISEQ (e.g., Illumina's Genome Analyzer; Genome Analyzer II; HISEQ 2000; HISEQ), SOLiD, Roche/454, PACBIO and/or SMRT, Helicos True Single Molecule Sequencing, Ion Torrent and Ion semiconductor-based sequencing (e.g., as developed by Life Technologies), WildFire, 5500, 5500xl W and/or 5500xl W Genetic Analyzer based technologies (e.g., as developed and sold by Life Technologies, U.S. Patent Application Publication No. 2013/0012399); Polony sequencing, Pyrosequencing, Massively Parallel Signature Sequencing (MPSS), RNA polymerase (RNAP) sequencing, LaserGen systems and methods, Nanopore-based platforms, chemical-sensitive field effect transistor (CHEMFET) array, electron microscopy-based sequencing (e.g., as developed by ZS Genetics, Halcyon Molecular), nanoball sequencing, the like or combinations thereof. Other sequencing methods that may be used to conduct methods herein include digital PCR, sequencing by hybridization, nanopore sequencing, chromosome-specific sequencing (e.g., using DANSR (digital analysis of selected regions) technology.

In some embodiments, nucleic acid is sequenced and the sequencing product (e.g., a collection of sequence reads) is processed prior to, or in conjunction with, an analysis of the sequenced nucleic acid. For example, sequence reads may be processed according to one or more of the following: aligning, mapping, filtering, counting, normalizing, weighting, generating a profile, and the like, and combinations thereof. Certain processing steps may be performed in any order and certain processing steps may be repeated.

Methods of the present disclosure can be used to reduce sequencing error rates. In some embodiments, prior to an initial denaturing, double-stranded molecules can be labeled with a barcode such that, after subsequent denaturing, single-stranded library preparation, and sequencing, sequences from nucleic acid molecules that were originally paired together can be associated. In some embodiments, after initial ligation of scaffold adapters, a pool of index primers is used to conduct index PCR such that copies are generated of both original sample nucleic acid molecules and nucleic acids from initial PCR first strand synthesis that both comprise the same barcode or UMI (or the complement thereof). By these or other means of associating strands that were originally hybridized (and therefore have complementary sequences), sequencing read information for both strands can be compared and used to reduce the sequencing error rate.

Mapping Reads

Sequence reads can be mapped and the number of reads mapping to a specified nucleic acid region (e.g., a chromosome or portion thereof) are referred to as counts. Any suitable mapping method (e.g., process, algorithm, program, software, module, the like or combination thereof) can be used. Certain aspects of mapping processes are described hereafter.

Mapping nucleotide sequence reads (i.e., sequence information from a fragment whose physical genomic position is unknown) can be performed in a number of ways, and often comprises alignment of the obtained sequence reads with a matching sequence in a reference genome. In such alignments, sequence reads generally are aligned to a reference sequence and those that align are designated as being “mapped,” as “a mapped sequence read” or as “a mapped read.” In certain embodiments, a mapped sequence read is referred to as a “hit” or “count.” In some embodiments, mapped sequence reads are grouped together according to various parameters and assigned to particular genomic portions, which are discussed in further detail below.

The terms “aligned,” “alignment,” or “aligning” generally refer to two or more nucleic acid sequences that can be identified as a match (e.g., 100% identity) or partial match. Alignments can be done manually or by a computer (e.g., a software, program, module, or algorithm), non-limiting examples of which include the Efficient Local Alignment of Nucleotide Data (ELAND) computer program distributed as part of the ILLUMINA Genomics Analysis pipeline. Alignment of a sequence read can be a 100% sequence match. In some instances, an alignment is less than a 100% sequence match (i.e., non-perfect match, partial match, partial alignment). In some embodiments an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment comprises a mismatch. In some embodiments, an alignment comprises 1, 2, 3, 4 or 5 mismatches. Two or more sequences can be aligned using either strand (e.g., sense or antisense strand). In certain embodiments a nucleic acid sequence is aligned with the reverse complement of another nucleic acid sequence.

Various computational methods can be used to map each sequence read to a portion. Non-limiting examples of computer algorithms that can be used to align sequences include, without limitation, BLAST, BLITZ, FASTA, BOWTIE 1, BOWTIE 2, ELAND, MAQ, PROBEMATCH, SOAP, BWA or SEQMAP, or variations thereof or combinations thereof. In some embodiments, sequence reads can be aligned with sequences in a reference genome. In some embodiments, sequence reads can be found and/or aligned with sequences in nucleic acid databases known in the art including, for example, GenBank, dbEST, dbSTS, EMBL (European Molecular Biology Laboratory) and DDBJ (DNA Databank of Japan). BLAST or similar tools can be used to search identified sequences against a sequence database. Search hits can then be used to sort the identified sequences into appropriate portions (described hereafter), for example.

In some embodiments, a read may uniquely or non-uniquely map to portions in a reference genome. A read is considered as “uniquely mapped” if it aligns with a single sequence in the reference genome. A read is considered as “non-uniquely mapped” if it aligns with two or more sequences in the reference genome. In some embodiments, non-uniquely mapped reads are eliminated from further analysis (e.g. quantification). A certain, small degree of mismatch (0-1) may be allowed to account for single nucleotide polymorphisms that may exist between the reference genome and the reads from individual samples being mapped, in certain embodiments. In some embodiments, no degree of mismatch is allowed for a read mapped to a reference sequence.

As used herein, the term “reference genome” can refer to any particular known, sequenced or characterized genome, whether partial or complete, of any organism or virus which may be used to reference identified sequences from a subject. For example, a reference genome used for human subjects as well as many other organisms can be found at the National Center for Biotechnology Information at World Wide Web URL ncbi.nlm.nih.gov. A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. In some embodiments, a reference genome comprises sequences assigned to chromosomes.

In certain embodiments, mappability is assessed for a genomic region (e.g., portion, genomic portion). Mappability is the ability to unambiguously align a nucleotide sequence read to a portion of a reference genome, typically up to a specified number of mismatches, including, for example, 0, 1, 2 or more mismatches. For a given genomic region, the expected mappability can be estimated using a sliding-window approach of a preset read length and averaging the resulting read-level mappability values. Genomic regions comprising stretches of unique nucleotide sequence sometimes have a high mappability value.

For paired-end sequencing, reads may be mapped to a reference genome by use of a suitable mapping and/or alignment program or algorithm, non-limiting examples of which include BWA (Li H. and Durbin R. (2009) Bioinformatics 25, 1754-60), Novoalign [Novocraft (2010)], Bowtie (Langmead B, et al., (2009) Genome Biol. 10:R25), SOAP2 (Li R, et al., (2009) Bioinformatics 25, 1966-67), BFAST (Homer N, et al., (2009) PLoS ONE 4, e7767), GASSST (Rizk, G. and Lavenier, D. (2010) Bioinformatics 26, 2534-2540), and MPscan (Rivals E., et al. (2009) Lecture Notes in Computer Science 5724, 246-260), and the like. Reads can be trimmed and/or merged by use of a suitable trimming and/or merging program or algorithm, non-limiting examples of which include Cutadapt, trimmomatic, SeqPrep, and usearch. Some paired-end reads, such as those from nucleic acid templates that are shorter than the sequencing read length, can have portions sequenced by both the forward read and the reverse read; in such instances, the forward and reverse reads can be merged into a single read using the overlap between the forward and reverse reads. Reads that do not overlap or that do not overlap sufficiently can remain unmerged and be mapped as paired reads. Paired-end reads may be mapped and/or aligned using a suitable short read alignment program or algorithm. Non-limiting examples of short read alignment programs include BarraCUDA, BFAST, BLASTN, BLAT, Bowtie, BWA, CASHX, CUDA-EC, CUSHAW, CUSHAW2, drFAST, ELAND, ERNE, GNUMAP, GEM, GensearchNGS, GMAP, Geneious Assembler, iSAAC, LAST, MAQ, mrFAST, mrsFAST, MOSAIK, MPscan, Novoalign, NovoalignCS, Novocraft, NextGENe, Omixon, PALMapper, Partek, PASS, PerM, QPalma, RazerS, REAL, cREAL, RMAP, rNA, RTG, Segemehl, SeqMap, Shrec, SHRiMP, SLIDER, SOAP, SOAP2, SOAP3, SOCS, SSAHA, SSAHA2, Stampy, SToRM, Subread, Subjunc, Taipan, UGENE, VelociMapper, TimeLogic, XpressAlign, ZOOM, the like or combinations thereof. Paired-end reads are often mapped to opposing ends of the same polynucleotide fragment, according to a reference genome. In some embodiments, read mates are mapped independently. In some embodiments, information from both sequence reads (i.e., from each end) is factored in the mapping process. A reference genome is often used to determine and/or infer the sequence of nucleic acids located between paired-end read mates. The term “discordant read pairs” as used herein refers to a paired-end read comprising a pair of read mates, where one or both read mates fail to unambiguously map to the same region of a reference genome defined, in part, by a segment of contiguous nucleotides. In some embodiments discordant read pairs are paired-end read mates that map to unexpected locations of a reference genome. Non-limiting examples of unexpected locations of a reference genome include (i) two different chromosomes, (ii) locations separated by more than a predetermined fragment size (e.g., more than 300 bp, more than 500 bp, more than 1000 bp, more than 5000 bp, or more than 10,000 bp), (iii) an orientation inconsistent with a reference sequence (e.g., opposite orientations), the like or a combination thereof. In some embodiments discordant read mates are identified according to a length (e.g., an average length, a predetermined fragment size) or expected length of template polynucleotide fragments in a sample. For example, read mates that map to a location that is separated by more than the average length or expected length of polynucleotide fragments in a sample are sometimes identified as discordant read pairs. Read pairs that map in opposite orientation are sometimes determined by taking the reverse complement of one of the reads and comparing the alignment of both reads using the same strand of a reference sequence. Discordant read pairs can be identified by any suitable method and/or algorithm known in the art or described herein (e.g., SVDetect, Lumpy, BreakDancer, BreakDancerMax, CREST, DELLY, the like or combinations thereof).

Sequence Read Quantification

Sequence reads that are mapped or partitioned based on a selected feature or variable can be quantified to determine the amount or number of reads that are mapped to one or more portions (e.g., portion of a reference genome). In certain embodiments, the quantity of sequence reads that are mapped to a portion or segment is referred to as a count or read density.

A count often is associated with a genomic portion. In some embodiments a count is determined from some or all of the sequence reads mapped to (i.e., associated with) a portion. In certain embodiments, a count is determined from some or all of the sequence reads mapped to a group of portions (e.g., portions in a segment or region).

A count can be determined by a suitable method, operation or mathematical process. A count sometimes is the direct sum of all sequence reads mapped to a genomic portion or a group of genomic portions corresponding to a segment, a group of portions corresponding to a sub-region of a genome (e.g., copy number variation region, copy number alteration region, copy number duplication region, copy number deletion region, microduplication region, microdeletion region, chromosome region, autosome region, sex chromosome region) and/or sometimes is a group of portions corresponding to a genome. A read quantification sometimes is a ratio, and sometimes is a ratio of a quantification for portion(s) in region a to a quantification for portion(s) in region b. Region a sometimes is one portion, segment region, copy number variation region, copy number alteration region, copy number duplication region, copy number deletion region, microduplication region, microdeletion region, chromosome region, autosome region and/or sex chromosome region. Region b independently sometimes is one portion, segment region, copy number variation region, copy number alteration region, copy number duplication region, copy number deletion region, microduplication region, microdeletion region, chromosome region, autosome region, sex chromosome region, a region including all autosomes, a region including sex chromosomes and/or a region including all chromosomes.

In some embodiments, a count is derived from raw sequence reads and/or filtered sequence reads. In certain embodiments a count is an average, mean or sum of sequence reads mapped to a genomic portion or group of genomic portions (e.g., genomic portions in a region). In some embodiments, a count is associated with an uncertainty value. A count sometimes is adjusted. A count may be adjusted according to sequence reads associated with a genomic portion or group of portions that have been weighted, removed, filtered, normalized, adjusted, averaged, derived as a mean, derived as a median, added, or combination thereof.

A sequence read quantification sometimes is a read density. A read density may be determined and/or generated for one or more segments of a genome. In certain instances, a read density may be determined and/or generated for one or more chromosomes. In some embodiments a read density comprises a quantitative measure of counts of sequence reads mapped to a segment or portion of a reference genome. A read density can be determined by a suitable process. In some embodiments a read density is determined by a suitable distribution and/or a suitable distribution function. Non-limiting examples of a distribution function include a probability function, probability distribution function, probability density function (PDF), a kernel density function (kernel density estimation), a cumulative distribution function, probability mass function, discrete probability distribution, an absolutely continuous univariate distribution, the like, any suitable distribution, or combinations thereof. A read density may be a density estimation derived from a suitable probability density function. A density estimation is the construction of an estimate, based on observed data, of an underlying probability density function. In some embodiments a read density comprises a density estimation (e.g., a probability density estimation, a kernel density estimation). A read density may be generated according to a process comprising generating a density estimation for each of the one or more portions of a genome where each portion comprises counts of sequence reads. A read density may be generated for normalized and/or weighted counts mapped to a portion or segment. In some instances, each read mapped to a portion or segment may contribute to a read density, a value (e.g., a count) equal to its weight obtained from a normalization process described herein. In some embodiments read densities for one or more portions or segments are adjusted. Read densities can be adjusted by a suitable method. For example, read densities for one or more portions can be weighted and/or normalized.

Reads quantified for a given portion or segment can be from one source or different sources. In one example, reads may be obtained from nucleic acid from a subject having cancer or suspected of having cancer. In such circumstances, reads mapped to one or more portions often are reads representative of both healthy cells (i.e., non-cancer cells) and cancer cells (e.g., tumor cells). In certain embodiments, some of the reads mapped to a portion are from cancer cell nucleic acid and some of the reads mapped to the same portion are from non-cancer cell nucleic acid. In another example, reads may be obtained from a nucleic acid sample from a pregnant female bearing a fetus. In such circumstances, reads mapped to one or more portions often are reads representative of both the fetus and the mother of the fetus (e.g., a pregnant female subject). In certain embodiments some of the reads mapped to a portion are from a fetal genome and some of the reads mapped to the same portion are from a maternal genome.

Classifications and Uses Thereof

Methods described herein can provide an outcome indicative of one or more characteristics of a sample or source described above. Methods described herein sometimes provide an outcome indicative of a phenotype and/or presence or absence of a medical condition for a test sample (e.g., providing an outcome determinative of the presence or absence of a medical condition and/or phenotype). An outcome often is part of a classification process, and a classification (e.g., classification of one or more characteristics of a sample or source; and/or presence or absence of a genotype, phenotype, genetic variation and/or medical condition for a test sample) sometimes is based on and/or includes an outcome. An outcome and/or classification sometimes is based on and/or includes a result of data processing for a test sample that facilitates determining one or more characteristics of a sample or source and/or presence or absence of a genotype, phenotype, genetic variation, genetic alteration, and/or medical condition in a classification process (e.g., a statistic value). An outcome and/or classification sometimes includes or is based on a score determinative of, or a call of, one or more characteristics of a sample or source and/or presence or absence of a genotype, phenotype, genetic variation, genetic alteration, and/or medical condition. In certain embodiments, an outcome and/or classification includes a conclusion that predicts and/or determines one or more characteristics of a sample or source and/or presence or absence of a genotype, phenotype, genetic variation, genetic alteration, and/or medical condition in a classification process.

Any suitable expression of an outcome and/or classification can be provided. An outcome and/or classification sometimes is based on and/or includes one or more numerical values generated using a processing method described herein in the context of one or more considerations of probability. Non-limiting examples of values that can be utilized include a sensitivity, specificity, standard deviation, median absolute deviation (MAD), measure of certainty, measure of confidence, measure of certainty or confidence that a value obtained for a test sample is inside or outside a particular range of values, measure of uncertainty, measure of uncertainty that a value obtained for a test sample is inside or outside a particular range of values, coefficient of variation (CV), confidence level, confidence interval (e.g., about 95% confidence interval), standard score (e.g., z-score), chi value, phi value, result of a t-test, p-value, ploidy value, fitted minority species fraction, area ratio, median level, the like or combination thereof. In some embodiments, an outcome and/or classification comprises a read density, a read density profile and/or a plot (e.g., a profile plot). In certain embodiments, multiple values are analyzed together, sometimes in a profile for such values (e.g., z-score profile, p-value profile, chi value profile, phi value profile, result of a t-test, value profile, the like, or combination thereof). A consideration of probability can facilitate determining one or more characteristics of a sample or source and/or whether a subject is at risk of having, or has, a genotype, phenotype, genetic variation and/or medical condition, and an outcome and/or classification determinative of the foregoing sometimes includes such a consideration.

In certain embodiments, an outcome and/or classification is based on and/or includes a conclusion that predicts and/or determines a risk or probability of the presence or absence of a genotype, phenotype, genetic variation and/or medical condition for a test sample. A conclusion sometimes is based on a value determined from a data analysis method described herein (e.g., a statistics value indicative of probability, certainty and/or uncertainty (e.g., standard deviation, median absolute deviation (MAD), measure of certainty, measure of confidence, measure of certainty or confidence that a value obtained for a test sample is inside or outside a particular range of values, measure of uncertainty, measure of uncertainty that a value obtained for a test sample is inside or outside a particular range of values, coefficient of variation (CV), confidence level, confidence interval (e.g., about 95% confidence interval), standard score (e.g., z-score), chi value, phi value, result of a t-test, p-value, sensitivity, specificity, the like or combination thereof). An outcome and/or classification sometimes is expressed in a laboratory test report for particular test sample as a probability (e.g., odds ratio, p-value), likelihood, or risk factor, associated with the presence or absence of a genotype, phenotype, genetic variation and/or medical condition. An outcome and/or classification for a test sample sometimes is provided as “positive” or “negative” with respect a particular genotype, phenotype, genetic variation and/or medical condition. For example, an outcome and/or classification sometimes is designated as “positive” in a laboratory test report for a particular test sample where presence of a genotype, phenotype, genetic variation and/or medical condition is determined, and sometimes an outcome and/or classification is designated as “negative” in a laboratory test report for a particular test sample where absence of a genotype, phenotype, genetic variation and/or medical condition is determined. An outcome and/or classification sometimes is determined and sometimes includes an assumption used in data processing.

There typically are four types of classifications generated in a classification process: true positive, false positive, true negative and false negative. The term “true positive” as used herein refers to presence of a genotype, phenotype, genetic variation, or medical condition correctly determined for a test sample. The term “false positive” as used herein refers to presence of a genotype, phenotype, genetic variation, or medical condition incorrectly determined for a test sample. The term “true negative” as used herein refers to absence of a genotype, phenotype, genetic variation, or medical condition correctly determined for a test sample. The term “false negative” as used herein refers to absence of a genotype, phenotype, genetic variation, or medical condition incorrectly determined for a test sample. Two measures of performance for a classification process can be calculated based on the ratios of these occurrences: (i) a sensitivity value, which generally is the fraction of predicted positives that are correctly identified as being positives; and (ii) a specificity value, which generally is the fraction of predicted negatives correctly identified as being negative.

In certain embodiments, a laboratory test report generated for a classification process includes a measure of test performance (e.g., sensitivity and/or specificity) and/or a measure of confidence (e.g., a confidence level, confidence interval). A measure of test performance and/or confidence sometimes is obtained from a clinical validation study performed prior to performing a laboratory test for a test sample. In certain embodiments, one or more of sensitivity, specificity and/or confidence are expressed as a percentage. In some embodiments, a percentage expressed independently for each of sensitivity, specificity or confidence level, is greater than about 90% (e.g., about 90, 91, 92, 93, 94, 95, 96, 97, 98 or 99%, or greater than 99% (e.g., about 99.5%, or greater, about 99.9% or greater, about 99.95% or greater, about 99.99% or greater)). A confidence interval expressed for a particular confidence level (e.g., a confidence level of about 90% to about 99.9% (e.g., about 95%)) can be expressed as a range of values, and sometimes is expressed as a range or sensitivities and/or specificities for a particular confidence level. Coefficient of variation (CV) in some embodiments is expressed as a percentage, and sometimes the percentage is about 10% or less (e.g., about 10, 9, 8, 7, 6, 5, 4, 3, 2 or 1%, or less than 1% (e.g., about 0.5% or less, about 0.1% or less, about 0.05% or less, about 0.01% or less)). A probability (e.g., that a particular outcome and/or classification is not due to chance) in certain embodiments is expressed as a standard score (e.g., z-score), a p-value, or result of a t-test. In some embodiments, a measured variance, confidence level, confidence interval, sensitivity, specificity and the like (e.g., referred to collectively as confidence parameters) for an outcome and/or classification can be generated using one or more data processing manipulations described herein.

An outcome and/or classification for a test sample often is ordered by, and often is provided to, a health care professional or other qualified individual (e.g., physician or assistant) who transmits an outcome and/or classification to a subject from whom the test sample is obtained. In certain embodiments, an outcome and/or classification is provided using a suitable visual medium (e.g., a peripheral or component of a machine, e.g., a printer or display). A classification and/or outcome often is provided to a healthcare professional or qualified individual in the form of a report. A report typically comprises a display of an outcome and/or classification (e.g., a value, one or more characteristics of a sample or source, or an assessment or probability of presence or absence of a genotype, phenotype, genetic variation and/or medical condition), sometimes includes an associated confidence parameter, and sometimes includes a measure of performance for a test used to generate the outcome and/or classification. A report sometimes includes a recommendation for a follow-up procedure (e.g., a procedure that confirms the outcome or classification). A report sometimes includes a visual representation of a chromosome or portion thereof (e.g., a chromosome ideogram or karyogram), and sometimes shows a visualization of a duplication and/or deletion region for a chromosome (e.g., a visualization of a whole chromosome for a chromosome deletion or duplication; a visualization of a whole chromosome with a deleted region or duplicated region shown; a visualization of a portion of chromosome duplicated or deleted; a visualization of a portion of a chromosome remaining in the event of a deletion of a portion of a chromosome) identified for a test sample.

A report can be displayed in a suitable format that facilitates determination of presence or absence of a genotype, phenotype, genetic variation and/or medical condition by a health professional or other qualified individual. Non-limiting examples of formats suitable for use for generating a report include digital data, a graph, a 2D graph, a 3D graph, and 4D graph, a picture (e.g., a jpg, bitmap (e.g., bmp), pdf, tiff, gif, raw, png, the like or suitable format), a pictograph, a chart, a table, a bar graph, a pie graph, a diagram, a flow chart, a scatter plot, a map, a histogram, a density chart, a function graph, a circuit diagram, a block diagram, a bubble map, a constellation diagram, a contour diagram, a cartogram, spider chart, Venn diagram, nomogram, and the like, or combination of the foregoing.

A report may be generated by a computer and/or by human data entry, and can be transmitted and communicated using a suitable electronic medium (e.g., via the internet, via computer, via facsimile, from one network location to another location at the same or different physical sites), or by another method of sending or receiving data (e.g., mail service, courier service and the like). Non-limiting examples of communication media for transmitting a report include auditory file, computer readable file (e.g., pdf file), paper file, laboratory file, medical record file, or any other medium described in the previous paragraph. A laboratory file or medical record file may be in tangible form or electronic form (e.g., computer readable form), in certain embodiments. After a report is generated and transmitted, a report can be received by obtaining, via a suitable communication medium, a written and/or graphical representation comprising an outcome and/or classification, which upon review allows a healthcare professional or other qualified individual to make a determination as to one or more characteristics of a sample or source, or presence or absence of a genotype, phenotype, genetic variation and/or or medical condition for a test sample.

An outcome and/or classification may be provided by and obtained from a laboratory (e.g., obtained from a laboratory file). A laboratory file can be generated by a laboratory that carries out one or more tests for determining one or more characteristics of a sample or source and/or presence or absence of a genotype, phenotype, genetic variation and/or medical condition for a test sample. Laboratory personnel (e.g., a laboratory manager) can analyze information associated with test samples (e.g., test profiles, reference profiles, test values, reference values, level of deviation, patient information) underlying an outcome and/or classification. For calls pertaining to presence or absence of a genotype, phenotype, genetic variation and/or medical condition that are close or questionable, laboratory personnel can re-run the same procedure using the same (e.g., aliquot of the same sample) or different test sample from a test subject. A laboratory may be in the same location or different location (e.g., in another country) as personnel assessing the presence or absence of a genotype, phenotype, genetic variation and/or a medical condition from the laboratory file. For example, a laboratory file can be generated in one location and transmitted to another location in which the information for a test sample therein is assessed by a healthcare professional or other qualified individual, and optionally, transmitted to the subject from which the test sample was obtained. A laboratory sometimes generates and/or transmits a laboratory report containing a classification of presence or absence of genomic instability, a genotype, phenotype, a genetic variation and/or a medical condition for a test sample. A laboratory generating a laboratory test report sometimes is a certified laboratory, and sometimes is a laboratory certified under the Clinical Laboratory Improvement Amendments (CLIA).

An outcome and/or classification sometimes is a component of a diagnosis for a subject, and sometimes an outcome and/or classification is utilized and/or assessed as part of providing a diagnosis for a test sample. For example, a healthcare professional or other qualified individual may analyze an outcome and/or classification and provide a diagnosis based on, or based in part on, the outcome and/or classification. In some embodiments, determination, detection or diagnosis of a medical condition, disease, syndrome or abnormality comprises use of an outcome and/or classification determinative of presence or absence of a genotype, phenotype, genetic variation and/or medical condition. Thus, provided herein are methods for diagnosing presence or absence of a genotype, phenotype, a genetic variation and/or a medical condition for a test sample according to an outcome or classification generated by methods described herein, and optionally according to generating and transmitting a laboratory report that includes a classification for presence or absence of the genotype, phenotype, a genetic variation and/or a medical condition for the test sample.

Machines, Software and Interfaces

Certain processes and methods described herein (e.g., selecting a subset of sequence reads, generating a sequence read profile, processing sequence read data, processing sequence read quantifications, determining one or more characteristics of a sample based on sequence read data or a sequence read profile) often are too complex for performing in the mind and cannot be performed without a computer, microprocessor, software, module or other machine. Methods described herein may be computer-implemented methods, and one or more portions of a method sometimes are performed by one or more processors (e.g., microprocessors), computers, systems, apparatuses, or machines (e.g., microprocessor-controlled machine).

Computers, systems, apparatuses, machines and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

Provided herein are computer readable storage media with an executable program stored thereon, where the program instructs a microprocessor to perform a method described herein. Provided also are computer readable storage media with an executable program module stored thereon, where the program module instructs a microprocessor to perform part of a method described herein. Also provided herein are systems, machines, apparatuses and computer program products that include computer readable storage media with an executable program stored thereon, where the program instructs a microprocessor to perform a method described herein. Provided also are systems, machines and apparatuses that include computer readable storage media with an executable program module stored thereon, where the program module instructs a microprocessor to perform part of a method described herein.

Also provided are computer program products. A computer program product often includes a computer usable medium that includes a computer readable program code embodied therein, the computer readable program code adapted for being executed to implement a method or part of a method described herein. Computer usable media and readable program code are not transmission media (i.e., transmission signals per se). Computer readable program code often is adapted for being executed by a processor, computer, system, apparatus, or machine.

In some embodiments, methods described herein (e.g., selecting a subset of sequence reads, generating a sequence read profile, processing sequence read data, processing sequence read quantifications, determining one or more characteristics of a sample based on sequence read data or a sequence read profile) are performed by automated methods. In some embodiments, one or more steps of a method described herein are carried out by a microprocessor and/or computer, and/or carried out in conjunction with memory. In some embodiments, an automated method is embodied in software, modules, microprocessors, peripherals and/or a machine comprising the like, that perform methods described herein. As used herein, software refers to computer readable program instructions that, when executed by a microprocessor, perform computer operations, as described herein.

Machines, software and interfaces may be used to conduct methods described herein. Using machines, software and interfaces, a user may enter, request, query or determine options for using particular information, programs or processes (e.g., processing sequence read data, processing sequence read quantifications, and/or providing an outcome), which can involve implementing statistical analysis algorithms, statistical significance algorithms, statistical algorithms, iterative steps, validation algorithms, and graphical representations, for example. In some embodiments, a data set may be entered by a user as input information, a user may download one or more data sets by suitable hardware media (e.g., flash drive), and/or a user may send a data set from one system to another for subsequent processing and/or providing an outcome (e.g., send sequence read data from a sequencer to a computer system for sequence read processing; send processed sequence read data to a computer system for further processing and/or yielding an outcome and/or report).

A system typically comprises one or more machines. Each machine comprises one or more of memory, one or more microprocessors, and instructions. Where a system includes two or more machines, some or all of the machines may be located at the same location, some or all of the machines may be located at different locations, all of the machines may be located at one location and/or all of the machines may be located at different locations. Where a system includes two or more machines, some or all of the machines may be located at the same location as a user, some or all of the machines may be located at a location different than a user, all of the machines may be located at the same location as the user, and/or all of the machine may be located at one or more locations different than the user.

A system sometimes comprises a computing machine and a sequencing apparatus or machine, where the sequencing apparatus or machine is configured to receive physical nucleic acid and generate sequence reads, and the computing apparatus is configured to process the reads from the sequencing apparatus or machine. The computing machine sometimes is configured to determine an outcome from the sequence reads (e.g., a characteristic of a sample).

A user may, for example, place a query to software which then may acquire a data set via internet access, and in certain embodiments, a programmable microprocessor may be prompted to acquire a suitable data set based on given parameters. A programmable microprocessor also may prompt a user to select one or more data set options selected by the microprocessor based on given parameters. A programmable microprocessor may prompt a user to select one or more data set options selected by the microprocessor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, machines, apparatuses, computer programs or a non-transitory computer-readable storage medium with an executable program stored thereon.

Systems addressed herein may comprise general components of computer systems, such as, for example, network servers, laptop systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, inkjet, impact, black and white or color printer), or other output useful for providing visual, auditory and/or hardcopy output of information (e.g., outcome and/or report).

In a system, input and output components may be connected to a central processing unit which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet based service where the user accesses a web page to enter and retrieve information. Accordingly, in certain embodiments, a system includes one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface may be used to receive signal information that can be detected by a signal detection module.

Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In some embodiments, output from a sequencing apparatus or machine may serve as data that can be input via an input device. In certain embodiments, sequence read information may serve as data that can be input via an input device. In certain embodiments, mapped sequence reads may serve as data that can be input via an input device. In certain embodiments, nucleic acid fragment size (e.g., length) may serve as data that can be input via an input device. In certain embodiments, output from a nucleic acid capture process (e.g., genomic region origin data) may serve as data that can be input via an input device. In certain embodiments, a combination of nucleic acid fragment size (e.g., length) and output from a nucleic acid capture process (e.g., genomic region origin data) may serve as data that can be input via an input device. In certain embodiments, simulated data is generated by an in silico process and the simulated data serves as data that can be input via an input device. The term “in silico” refers to research and experiments performed using a computer. In silico processes include, but are not limited to, mapping sequence reads and processing mapped sequence reads according to processes described herein.

A system may include software useful for performing a process or part of a process described herein, and software can include one or more modules for performing such processes (e.g., sequencing module, logic processing module, data display organization module). The term “software” refers to computer readable program instructions that, when executed by a computer, perform computer operations. Instructions executable by the one or more microprocessors sometimes are provided as executable code, that when executed, can cause one or more microprocessors to implement a method described herein. A module described herein can exist as software, and instructions (e.g., processes, routines, subroutines) embodied in the software can be implemented or performed by a microprocessor. For example, a module (e.g., a software module) can be a part of a program that performs a particular process or task. The term “module” refers to a self-contained functional unit that can be used in a larger machine or software system. A module can comprise a set of instructions for carrying out a function of the module. A module can transform data and/or information. Data and/or information can be in a suitable form. For example, data and/or information can be digital or analogue. In certain embodiments, data and/or information sometimes can be packets, bytes, characters, or bits. In some embodiments, data and/or information can be any gathered, assembled or usable data or information. Non-limiting examples of data and/or information include a suitable media, pictures, video, sound (e.g. frequencies, audible or non-audible), numbers, constants, a value, objects, time, functions, instructions, maps, references, sequences, reads, mapped reads, levels, ranges, thresholds, signals, displays, representations, or transformations thereof. A module can accept or receive data and/or information, transform the data and/or information into a second form, and provide or transfer the second form to a machine, peripheral, component or another module. A microprocessor can, in certain embodiments, carry out the instructions in a module. In some embodiments, one or more microprocessors are required to carry out instructions in a module or group of modules. A module can provide data and/or information to another module, machine or source and can receive data and/or information from another module, machine or source.

A computer program product sometimes is embodied on a tangible computer-readable medium, and sometimes is tangibly embodied on a non-transitory computer-readable medium. A module sometimes is stored on a computer readable medium (e.g., disk, drive) or in memory (e.g., random access memory). A module and microprocessor capable of implementing instructions from a module can be located in a machine or in a different machine. A module and/or microprocessor capable of implementing an instruction for a module can be located in the same location as a user (e.g., local network) or in a different location from a user (e.g., remote network, cloud system). In embodiments in which a method is carried out in conjunction with two or more modules, the modules can be located in the same machine, one or more modules can be located in different machine in the same physical location, and one or more modules may be located in different machines in different physical locations.

A machine, in some embodiments, comprises at least one microprocessor for carrying out the instructions in a module. Sequence read quantifications (e.g., counts) sometimes are accessed by a microprocessor that executes instructions configured to carry out a method described herein. Sequence read quantifications that are accessed by a microprocessor can be within memory of a system, and the sequence read counts can be accessed and placed into the memory of the system after they are obtained. In some embodiments, a machine includes a microprocessor (e.g., one or more microprocessors) which microprocessor can perform and/or implement one or more instructions (e.g., processes, routines and/or subroutines) from a module. In some embodiments, a machine includes multiple microprocessors, such as microprocessors coordinated and working in parallel. In some embodiments, a machine operates with one or more external microprocessors (e.g., an internal or external network, server, storage device and/or storage network (e.g., a cloud)). In some embodiments, a machine comprises a module (e.g., one or more modules). A machine comprising a module often is capable of receiving and transferring one or more of data and/or information to and from other modules.

In certain embodiments, a machine comprises peripherals and/or components. In certain embodiments, a machine can comprise one or more peripherals or components that can transfer data and/or information to and from other modules, peripherals and/or components. In certain embodiments, a machine interacts with a peripheral and/or component that provides data and/or information. In certain embodiments, peripherals and components assist a machine in carrying out a function or interact directly with a module. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., ipads, tablets), touch screens, smart phones, mobile phones, USB I/O devices, USB mass storage devices, keyboards, a computer mouse, digital pens, modems, hard drives, jump drives, flash drives, a microprocessor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, WiFi, and the like), the world wide web (www), the internet, a computer and/or another module.

Software often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash memory devices (e.g., flash drives), RAM, floppy discs, the like, and other such media on which the program instructions can be recorded. In online implementation, a server and web site maintained by an organization can be configured to provide software downloads to remote users, or remote users may access a remote system maintained by an organization to remotely access software. Software may obtain or receive input information. Software may include a module that specifically obtains or receives data (e.g., a data receiving module that receives sequence read data and/or mapped read data) and may include a module that specifically processes the data (e.g., a processing module that processes received data (e.g., filters, normalizes, provides an outcome and/or report). The terms “obtaining” and “receiving” input information refers to receiving data (e.g., sequence reads, mapped reads) by computer communication means from a local, or remote site, human data entry, or any other method of receiving data. The input information may be generated in the same location at which it is received, or it may be generated in a different location and transmitted to the receiving location. In some embodiments, input information is modified before it is processed (e.g., placed into a format amenable to processing (e.g., tabulated)).

Software can include one or more algorithms in certain embodiments. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational genometric algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, Fortran, and the like. In some embodiments, an algorithm can be configured or modified to include margin of errors, statistical analysis, statistical significance, and/or comparison to other information or data sets (e.g., applicable when using a neural net or clustering algorithm).

In certain embodiments, several algorithms may be implemented for use in software. These algorithms can be trained with raw data in some embodiments. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity, in some embodiments. An algorithm with the highest sensitivity and/or specificity may be identified and utilized, in certain embodiments.

In certain embodiments, simulated (or simulation) data can aid data processing, for example, by training an algorithm or testing an algorithm. In some embodiments, simulated data includes hypothetical various samplings of different groupings of sequence reads. Simulated data may be based on what might be expected from a real population or may be skewed to test an algorithm and/or to assign a correct classification. Simulated data also is referred to herein as “virtual” data. Simulations can be performed by a computer program in certain embodiments. One possible step in using a simulated data set is to evaluate the confidence of identified results, e.g., how well a random sampling matches or best represents the original data. One approach is to calculate a probability value (p-value), which estimates the probability of a random sample having better score than the selected samples. In some embodiments, an empirical model may be assessed, in which it is assumed that at least one sample matches a reference sample (with or without resolved variations). In some embodiments, another distribution, such as a Poisson distribution for example, can be used to define the probability distribution.

A system may include one or more microprocessors in certain embodiments. A microprocessor can be connected to a communication bus. A computer system may include a main memory, often random access memory (RAM), and can also include a secondary memory. Memory in some embodiments comprises a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A microprocessor may implement software in a system. In some embodiments, a microprocessor may be programmed to automatically perform a task described herein that a user could perform. Accordingly, a microprocessor, or algorithm conducted by such a microprocessor, can require little to no supervision or input from a user (e.g., software may be programmed to implement a function automatically). In some embodiments, the complexity of a process is so large that a single person or group of persons could not perform the process in a timeframe short enough for determining one or more characteristics of a sample.

In some embodiments, secondary memory may include other similar means for allowing computer programs or other instructions to be loaded into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data to be transferred from the removable storage unit to a computer system.

Provided herein, in certain embodiments, are systems, machines and apparatuses comprising one or more microprocessors and memory, which memory comprises sequence reads generated from a nucleic acid library produced from a test sample, and which memory comprises instructions executable by the one or more microprocessors and which instructions executable by the one or more microprocessors are configured to a) determine nucleic acid fragment lengths from the sequence reads, and b) analyze the nucleic acid fragment lengths.

Provided herein, in certain embodiments, are machines comprising one or more microprocessors and memory, which memory comprises instructions executable by the one or more microprocessors and which memory comprises sequence reads generated from a nucleic acid library produced from a test sample, and which instructions executable by the one or more microprocessors are configured to a) determine nucleic acid fragment lengths from the sequence reads, and b) analyze the nucleic acid fragment lengths.

Provided herein, in certain embodiments, are non-transitory computer-readable storage media with an executable program stored thereon, where the program instructs a microprocessor to perform the following: a) access sequence reads generated from a nucleic acid library produced from a test sample, b) determine nucleic acid fragment lengths from the sequence reads, and c) analyze the nucleic acid fragment lengths.

Provided herein, in certain embodiments, are systems, machines and apparatuses comprising one or more microprocessors and memory, which memory comprises instructions executable by the one or more microprocessors and which instructions executable by the one or more microprocessors are configured to a) determine lengths of nucleic acid fragments in a test sample from a subject, and b) detect the presence or absence of a population of cells in a subject according the nucleic acid fragment lengths determined in (a).

Provided herein, in certain embodiments, are machines comprising one or more microprocessors and memory, which memory comprises instructions executable by the one or more microprocessors and which instructions executable by the one or more microprocessors are configured to a) determine lengths of nucleic acid fragments in a test sample from a subject, and b) detect the presence or absence of a population of cells in a subject according the nucleic acid fragment lengths determined in (a).

Provided herein, in certain embodiments, are non-transitory computer-readable storage media with an executable program stored thereon, where the program instructs a microprocessor to perform the following: a) determine lengths of nucleic acid fragments in a test sample from a subject, and b) detect the presence or absence of a population of cells in a subject according the nucleic acid fragment lengths determined in (a).

Methods for Analyzing Nucleic Acids

Provided herein are methods for analyzing nucleic acids. Nucleic acid be analyzed according to an assessment of fragment length. Fragment length may be determined using any suitable method for determining fragment length. In some embodiments, fragment length is determined according to the length of a single-end sequencing read (e.g., where the read length covers the length of the entire fragment). In some embodiments, fragment length is determined according to mapped positions of paired-end sequencing reads. In some embodiments, the purity and/or quality of nucleic acid is assessed according to a fragment length profile. In some embodiments, one or more A/B transitions are identified in a genome according to a fragment length profile. In some embodiments, one or more genomic regions are identified as being in an A compartment or as being in a B compartment according to a fragment length profile. In some embodiments, presence or absence of a disease is determined according to a fragment length profile. A fragment length profile may include quantifications of fragments having particular lengths. In some embodiments, the purity and/or quality of nucleic acid, or the presence/absence of disease, is assessed according to an amount of a major nucleic acid species and an amount of a minor nucleic acid species in the fragment length profile. A major species generally refers to the fragment length most abundant in the sample. A major species may refer to the intended or expected fragment length of the nucleic acid being assessed. For example, for an oligonucleotide designed to include exactly 50 nucleotides, an assessment of the purity and/or quality of that oligonucleotide may yield a major species length of 50 nucleotides. A minor species generally refers to the remaining fragment lengths that are not the major species. A minor species may refer to the unintended or unexpected fragment lengths of the nucleic acid being assessed. For example, for an oligonucleotide designed to include exactly 50 nucleotides, an assessment of the purity and/or quality of that oligonucleotide may yield a minor species having lengths greater than 50 and/or less than 50, but not exactly 50 nucleotides. The purity and/or quality of nucleic acid may be expressed as a ratio or percentage. For example, an oligonucleotide may be considered 90% pure for the major species if 90% of the oligonucleotides in the sample are of the major species fragment length and 10% of the oligonucleotides in the sample (collectively) are of minor species fragment length.

Kits

Provided in certain embodiments are kits. The kits may include any components and compositions described herein (e.g., scaffold adapters and components/subcomponents thereof, oligonucleotides, oligonucleotide components/regions, scaffold polynucleotides, scaffold polynucleotide components/regions, nucleic acids, single-stranded nucleic acids, primers, single-stranded binding proteins, enzymes) useful for performing any of the methods described herein, in any suitable combination. Kits may further include any reagents, buffers, or other components useful for carrying out any of the methods described herein. For example, a kit may include one or more of a plurality of scaffold adapter species, a plurality of oligonucleotide species, and/or a plurality of scaffold polynucleotide species, a kinase adapted to 5′ phosphorylate nucleic acids (e.g., a polynucleotide kinase (PNK)), a DNA ligase, and any combination thereof. In some embodiments, a kit further comprises one or more of a reverse transcriptase, a polymerase, single stranded binding proteins (SSBs), a primer oligonucleotide, an RNAse, a ligase (e.g., T4 RNA ligase 1, T4 RNA ligase 2, T4 DNA ligase), one or more distinctive nucleotides, a hairpin adapter, and the like.

Kits may include components for capturing single-stranded DNA and/or single-stranded RNA. Kits for capturing single-stranded DNA may be configured such that a user provides double-stranded or single-stranded DNA. Kits for capturing single-stranded RNA may be configured such that a user provides cDNA (either single or double stranded), or provides RNA (e.g., total RNA or rRNA-depleted RNA). A kit for capturing single-stranded RNA may include rRNA depletion reagents, mRNA enrichment reagents, fragmentation reagents, cDNA synthesis reagents, and/or RNA digestion reagents.

Components of a kit may be present in separate containers, or multiple components may be present in a single container. Suitable containers include a single tube (e.g., vial), one or more wells of a plate (e.g., a 96-well plate, a 384-well plate, and the like), and the like.

Kits may also comprise instructions for performing one or more methods described herein and/or a description of one or more components described herein. For example, a kit may include instructions for using scaffold adapters described herein, or components thereof, to capture single-stranded nucleic acid fragments and/or to produce a nucleic acid library. Instructions and/or descriptions may be in printed form and may be included in a kit insert. In some embodiments, instructions and/or descriptions are provided as an electronic storage data file present on a suitable computer readable storage medium, e.g., portable flash drive, DVD, CD-ROM, diskette, and the like. A kit also may include a written description of an internet location that provides such instructions or descriptions.

Certain Implementations

Following are non-limiting examples of certain implementations of the technology.
A1. A method for analyzing nucleic acid fragment length in a test sample, comprising:
   a) producing a nucleic acid library from the test sample;
   b) sequencing the nucleic acid library, thereby generating sequence reads;
   c) determining nucleic acid fragment lengths from the sequence reads; and
   d) analyzing the nucleic acid fragment lengths.
A2. The method of embodiment A1, wherein (a) comprises use of a single-stranded nucleic acid library preparation method.
A3. The method of embodiment A2, wherein the single-stranded nucleic acid library preparation method comprises combining (i) a nucleic acid composition comprising single-stranded nucleic acid (ssNA), (ii) a first oligonucleotide, and (iii) a plurality of first scaffold polynucleotide species, wherein:
   the nucleic acid composition is from the test sample;
   each polynucleotide in the plurality of first scaffold polynucleotide species comprises an ssNA hybridization region and a first oligonucleotide hybridization region, and
   the nucleic acid composition, the first oligonucleotide, and the plurality of first scaffold polynucleotide species are combined under conditions in which a molecule of the first scaffold polynucleotide species is hybridized to (1) a first ssNA terminal region and (2) a molecule of the first oligonucleotide, thereby forming hybridization products in which an end of the molecule of the first oligonucleotide is adjacent to an end of the first ssNA terminal region.
A4. The method of embodiment A2, wherein the single-stranded nucleic acid library preparation method comprises combining (i) a nucleic acid composition comprising single-stranded nucleic acid (ssNA), (ii) a plurality of first oligonucleotide species, and (iii) a plurality of first scaffold polynucleotide species, wherein:
   the nucleic acid composition is from the test sample;
   each polynucleotide in the plurality of first scaffold polynucleotide species comprises an ssNA hybridization region and a first oligonucleotide hybridization region, and
   the nucleic acid composition, the plurality of first oligonucleotide species, and the plurality of first scaffold polynucleotide species are combined under conditions in which a molecule of the first scaffold polynucleotide species is hybridized to (1) a first ssNA terminal region and (2) a molecule of the first oligonucleotide species, thereby forming hybridization products in which an end of the molecule of the first oligonucleotide is adjacent to an end of the first ssNA terminal region.
A5. The method of embodiment A4, wherein each oligonucleotide in the plurality of first oligonucleotide species comprises a first unique molecular identifier (UMI) flanked by a first flank region and a second flank region.
A6. The method of embodiment A5, wherein the first oligonucleotide hybridization region comprises (i) a polynucleotide complementary to the first flank region, and (ii) a polynucleotide complementary to the second flank region.
A7. The method of any one of embodiments A3 to A6, wherein prior to the combining, each of the first scaffold polynucleotide species is hybridized to the first oligonucleotide to form a plurality of first scaffold duplex species.
A8. The method of any one of embodiments A3 to A7, further comprising covalently linking the adjacent ends of the first oligonucleotide and the first ssNA terminal region, thereby generating covalently linked hybridization products.
A9. The method of embodiment A8, wherein the covalently linking comprises contacting the hybridization products with an agent comprising a ligase activity under conditions in which an end of the first ssNA terminal region is covalently linked to an end of the first oligonucleotide.
A10. The method of any one of embodiments A1 to A9, wherein the sequencing in (b) comprises massively parallel sequencing.
A11. The method of any one of embodiments A1 to A10, wherein the sequencing in (b) comprises single-end sequencing.
A12. The method of any one of embodiments A1 to A10, wherein the sequencing in (b) comprises paired-end sequencing.
A13. The method of any one of embodiments A1 to A12, wherein the sequencing in (b) generates thousands of sequence reads.
A14. The method of any one of embodiments A1 to A12, wherein the sequencing in (b) generates millions of sequence reads.
A15. The method of any one of embodiments A1 to A12, wherein the sequencing in (b) generates thousands to millions of sequence reads.
A16. The method of any one of embodiments A1 to A15, wherein the nucleic acid fragment lengths in (c) are determined according to the length of a single-end sequencing read.
A17. The method of any one of embodiments A1 to A15, wherein the nucleic acid fragment lengths in (c) are determined according to mapped positions of paired-end sequencing reads.
A18. The method of any one of embodiments A1 to A17, wherein the analyzing in (d) comprises generating fragment counts.
A19. The method of any one of embodiments A1 to A18, wherein the analyzing in (d) comprises generating one or more fragment length ratios.
A20. The method of embodiment A19, wherein each of the one or more fragment length ratios is a ratio of short fragment counts to long fragment counts.
A21. The method of embodiment A20, wherein the short fragments comprise fragments that are about 100 bp to about 149 bp in length, and the long fragments comprise fragments that are greater than about 150 bp in length.
A22. The method of any one of embodiments A19 to A21, wherein the one or more fragment length ratios are generated for one or more genomic portions.
A23. The method of embodiment A22, wherein the one or more genomic portions are 5 mb in length.
A24. The method of any one of embodiments A19 to A23, further comprising generating a fragment length ratio profile.
A25. The method of any one of embodiments A19 to A24, further comprising determining an average fragment length ratio profile.
A26. The method of any one of embodiments A19 to A25, further comprising normalizing the one or more fragment length ratios or the fragment length ratio profile.
A27. The method of embodiment A26, wherein the normalizing comprises a guanine and cytosine (GC) bias correction.
A28. The method of embodiment A26 or A27, wherein the normalizing comprises generating one or more z-scores.
A29. The method of any one of embodiments A26 to A28, wherein the normalizing comprises determining a log likelihood.
A30. The method of any one of embodiments A26 to A29, wherein the normalizing comprises determining a log likelihood of z-scores.
A31. The method of any one of embodiments A1 to A30, wherein the test sample comprises cell-free DNA (cfDNA).
A32. The method of any one of embodiments A1 to A31, wherein the test sample comprises circulating tumor DNA (ctDNA).
A33. The method of any one of embodiments A1 to A33, further comprising comparing the fragment length analysis in (d) for the test sample to a fragment length analysis for one or more control samples, thereby generating a comparison.
A34. The method of embodiment A33, further comprising determining the presence or absence of a disease, or a disease subtype, for the test sample according to the comparison.
A35. The method of any one of embodiments A1 to A32, wherein the test sample is obtained from a test subject.
A36. The method of any one of embodiments A33 to A35, wherein the one or more control samples are obtained from one or more healthy subjects and/or one or more diseased subjects.
A37. The method of any one of embodiments A34 to A36, wherein the disease is cancer.
A38. The method of any one of embodiments A34 to A37, wherein the disease is acute myeloid leukemia (AML).
A39. The method of any one of embodiments A34 to A37, wherein the disease is myelodysplastic syndrome (MDS).
A39.1 The method of any one of embodiments A34 to A37, wherein the disease is MDS with multiple lineage dysplasia and ring sideroblasts (MDS-RS-MLD).
A39.2 The method of any one of embodiments A34 to A37, wherein the disease subtype is MDS with multiple lineage dysplasia and ring sideroblasts (MDS-RS-MLD).
A39.3 The method of embodiment A39.2, comprising comparing the fragment length analysis in (d) for the test sample to a fragment length analysis for one or more control samples, thereby generating a comparison, wherein the test sample is obtained from a test subject having, or suspected of having, MDS-RS-MLD, and the one or more control samples are obtained from one or more healthy subjects and/or one or more subjects having MDS.
A40. The method of any one of embodiments A1 to A39.3, comprising after (a) and prior to (b), enriching the library for one or more genome regions of interest or one or more exome regions of interest.
A41. The method of embodiment A40, wherein the one or more genome regions of interest or the one or more exome regions of interest comprise known variants.
A42. The method of embodiment A41, wherein the known variants comprise known cancer variants.
A43. The method of any one of embodiments A40 to A42, wherein the enriching comprises hybridizing capture probes to the library, wherein the capture probes are specific to the one or more genome regions of interest or the one or more exome regions of interest.
A44. The method of any one of embodiments, A1 to A43, wherein the analyzing in (d) comprises identifying one or more A/B transitions in the genome of the test sample according to the fragment lengths determined in (c).
A45. The method of embodiment A44, wherein the identifying one or more A/B transitions in the genome of the test sample according to the fragment lengths determined in (c) comprises generating one or more fragment length ratios.
A46. The method of embodiment A45, wherein each of the one or more fragment length ratios is a ratio chosen from short fragment counts to long fragment counts, short fragment counts to medium fragment counts, and medium fragment counts to long fragment counts.
A47. The method of embodiment A46, wherein the short fragments comprise fragments that are about 0 bp to about 99 bp in length, the medium fragments comprise fragments that are about 100 bp to about 149 bp in length, and the long fragments comprise fragments that are about 150 bp to about 220 bp in length.
A48. The method of any one of embodiments A45 to A47, wherein the one or more fragment length ratios are generated for one or more genomic portions.
A49. The method of embodiment A48, wherein the one or more genomic portions are 100 kb in length.
A49.1 The method of embodiment A48, wherein the one or more genomic portions are 250 kb in length.
A50. The method of any one of embodiments A44 to A49.1, wherein the analyzing in (d) further comprises comparing one or more A/B transitions identified in the genome of the test sample to one or more A/B transition aggregates, thereby generating an A/B transition comparison.
A51. The method of embodiment A50, wherein the one or more A/B transition aggregates are generated from a plurality of healthy samples and/or a plurality of diseased samples.
A52. The method of embodiment A51, wherein the diseased samples comprise cancer samples.
A53. The method of any one of embodiments A50 to A53, further comprising identifying the presence or absence of a disease for the test sample according to the A/B transition comparison.
A54. The method of embodiment A53, wherein the disease is cancer.
A55. The method of any one of embodiments A1 to A43, wherein the analyzing in (d) comprises identifying one or more regions in the genome of the test sample as belonging to an A compartment or as belonging to a B compartment according to the fragment lengths determined in (c).
A56. The method of embodiment A55, wherein the identifying one or more regions in the genome of the test sample as belonging to an A compartment or as belonging to a B compartment according to the fragment lengths determined in (c) comprises generating one or more fragment length ratios.
A57. The method of embodiment A56, wherein each of the one or more fragment length ratios is a ratio chosen from short fragment counts to long fragment counts, short fragment counts to medium fragment counts, and medium fragment counts to long fragment counts.
A58. The method of embodiment A57, wherein the short fragments comprise fragments that are about 0 bp to about 99 bp in length, the medium fragments comprise fragments that are about 100 bp to about 149 bp in length, and the long fragments comprise fragments that are about 150 bp to about 220 bp in length.
A59. The method of any one of embodiments A55 to A58, wherein the one or more fragment length ratios are generated for one or more genomic portions.
A60. The method of embodiment A59, wherein the one or more genomic portions are 100 kb in length.
A60.1 The method of embodiment A59, wherein the one or more genomic portions are 250 kb in length.
A61. The method of any one of embodiments A55 to A60.1, wherein the analyzing in (d) further comprises comparing an A compartment status or a B compartment status of the one or more regions in the genome of the test sample to one or more A/B compartment status aggregates, thereby generating an A/B compartment status comparison.
A62. The method of embodiment A61, wherein the one or more A/B compartment status aggregates are generated from a plurality of healthy samples and/or a plurality of diseased samples.
A63. The method of embodiment A62, wherein the diseased samples comprise cancer samples.
A64. The method of any one of embodiments A61 to A63, further comprising identifying the presence or absence of a disease, or a disease subtype, for the test sample according to the A/B compartment status comparison.
A65. The method of embodiment A64, wherein the disease is cancer.
A66. The method of embodiment A65, wherein the disease is MDS.
A67. The method of embodiment A65, wherein the disease is MDS-RS-MLD.
B1. A method for detecting the presence or absence of a population of cells in a subject comprising:
   a) determining lengths of nucleic acid fragments in a test sample from a subject; and
   b) detecting the presence or absence of a population of cells in a subject according the nucleic acid fragment lengths determined in (a).
B1.1 The method of embodiment B1, further comprising obtaining nucleic acid sequence reads of the nucleic acid fragments.
B1.2 The method of embodiment B1.1, wherein the nucleic acid fragment lengths are determined from the nucleic acid sequence reads.
B2. The method of embodiment B1.1 or 1.2, comprising prior to (a) producing a nucleic acid library from the test sample.
B3. The method of embodiment B2, wherein producing a nucleic acid library comprises use of a single-stranded nucleic acid library preparation method.
B4. The method of embodiment B3, wherein the single-stranded nucleic acid library preparation method comprises one or more features of embodiments A3 to A9.
B5. The method of any one of embodiments B1-B4, wherein the nucleic acid sequence reads in are generated by a sequencing process.
B6. The method of any one of embodiments B1-B5, comprising prior to (a) sequencing nucleic acids in the test sample by a sequencing process, thereby generating the nucleic acid sequence reads.
B7. The method of embodiment B5 or B6, wherein the sequencing process comprises massively parallel sequencing.
B8. The method of any one of embodiments B5-B7, wherein the sequencing process is a genome-wide sequencing process.
B9. The method of any one of embodiments B5-B8, wherein the sequencing process is a non-targeted sequencing process.
B10. The method of any one of embodiments B5-B9, wherein the sequencing process comprises single-end sequencing.
B11. The method of embodiment B10, wherein the nucleic acid fragment lengths are determined according to the length of a single-end sequencing read.
B12. The method of any one of embodiments B5-B9, wherein the sequencing process comprises paired-end sequencing.
B13. The method of embodiment B12, wherein the nucleic acid fragment lengths are determined according to mapped positions of paired-end sequencing reads.
B14. The method of any one of embodiments B5-B13, wherein the sequencing process generates thousands of sequence reads.
B15. The method of any one of embodiments B5-B13, wherein the sequencing process generates millions of sequence reads.
B16. The method of any one of embodiments B5-B13, wherein the sequencing process generates thousands to millions of sequence reads.
B17. The method of any one of embodiments B5-B13, wherein the sequencing process generates between about 100,000 reads to about 1 billion reads.
B17.1 The method of any one of embodiments B5-B13, wherein the sequencing process generates between about 1 million reads to about 10 million reads.
B18. The method of any one of embodiments B5-B13 wherein the sequencing process generates about 5 million reads.
B18.1 The method of any one of embodiments B5-B13 wherein the sequencing process generates about 1 million reads.
B18.2 The method of any one of embodiments B5-B13 wherein the sequencing process generates about 500,000 reads.
B19. The method of any one of embodiments B5-B18.2, wherein the sequencing process is performed at about 0.01-fold coverage to about 1-fold coverage.
B19.1 The method of any one of embodiments B5-18.2, wherein the sequencing process is performed at about 0.02-fold coverage.
B19.2 The method of any one of embodiments B5-B18.2, wherein the sequencing process is performed at about 0.05-fold coverage.
B19.3 The method of any one of embodiments B5-B18.2, wherein the sequencing process is performed at about 0.1-fold coverage.
B19.4 The method of any one of embodiments B5-B18.2, wherein the sequencing process is performed at about 1-fold coverage to about 30-fold coverage.
B19.5 The method of any one of embodiments B5-18.2, wherein the sequencing process is performed at about 5-fold coverage.
B19.5 The method of any one of embodiments B5-B18.2, wherein the sequencing process is performed at a coverage of at least about 0.01-fold.
B20. The method of embodiment B1, wherein the nucleic acid fragment lengths are determined according to a method comprising capillary electrophoresis.
B21. The method of embodiment B20, further comprising prior to (a) performing capillary electrophoresis on the nucleic acid fragments in the test sample.
B22. The method of any one of embodiments B1-B21, further comprising after (a) generating fragment counts.
B23. The method of embodiment B22, wherein detecting the presence or absence of a population of cells in a subject in (b) is according to the fragment counts.
B24. The method of any one of embodiments B1-B23, further comprising after (a) generating one or more fragment length ratios.
B25. The method of embodiment B24, wherein each of the one or more fragment length ratios is a ratio chosen from short fragment counts to long fragment counts, short fragment counts to medium fragment counts, and medium fragment counts to long fragment counts.
B26. The method of embodiment B25, wherein the short fragments comprise fragments that are about 0 bp to about 99 bp in length, the medium fragments comprise fragments that are about 100 bp to about 149 bp in length, and the long fragments comprise fragments that are about 150 bp to about 220 bp in length.
B27. The method of any one of embodiments B24-B26, wherein the one or more fragment length ratios are generated for one or more genomic portions.
B28. The method of embodiment B27, wherein the one or more genomic portions are about 50 kb to about 500 kb in length.
B29. The method of embodiment B27, wherein the one or more genomic portions are about 250 kb in length.
B30. The method of any one of embodiments B24-B29, wherein detecting the presence or absence of a population of cells in a subject in (b) is according to the one or more fragment length ratios.
B31. The method of any one of embodiments B1-B30, further comprising comparing the nucleic acid fragment lengths, or a derivative thereof, for the test sample to nucleic acid fragment lengths, or a derivative thereof, for one or more control samples, thereby generating a comparison.
B31.1 The method of embodiment B31, wherein the comparison comprises a t-test.
B31.2 The method of embodiment B31, wherein the comparison comprises a regression analysis.
B32. The method of any one of embodiments B31-B31.2, wherein the one or more control samples are obtained from one or more healthy subjects.
B33. The method of any one of embodiments B31-B31.2, wherein the one or more control samples are obtained from one or more diseased subjects.
B34. The method of any one of embodiments B31-B33, wherein detecting the presence or absence of a population of cells in a subject in (b) is according to the comparison.
B35. The method of any one of embodiments B1-B34, further comprising enriching the test sample for one or more selected genomic regions, thereby generating one or more pools of enriched nucleic acid.
B36. The method of embodiment B35, wherein the one or more genomic regions are selected according to A/B status for a chosen cell type.
B37. The method of embodiment B36, wherein the one or more genomic regions are selected according to A/B status differences between a diseased cell and a non-diseased cell.
B38. The method of any one of embodiments B35-B37, wherein the enriching is performed prior to (a).
B39. The method of any one of embodiments B35-B38, wherein the enriching comprises hybridizing capture probes to nucleic acid fragments in the test sample, wherein the capture probes are specific to the one or more selected genomic regions.
B40. The method of any one of embodiments 1 to B39, further comprising identifying an A/B status of the genome, or portion thereof, for the test sample according to the nucleic acid fragment lengths determined in (a).
B41. The method of embodiment B40, further comprising comparing the A/B status of the genome, or portion thereof, for the test sample to an A/B status of a control genome, thereby generating an A/B status comparison.
B41.1 The method of embodiment B41, wherein the comparison comprises a t-test.
B41.2 The method of embodiment B41, wherein the comparison comprises a regression analysis.
B42. The method of any one of embodiments B41-B41.2, wherein the control genome is from one or more non-diseased cells.
B43. The method of any one of embodiments B41-B41.2, wherein the control genome is from one or more diseased cells.
B44. The method of any one of embodiments B41-B43, wherein detecting the presence or absence of a population of cells in a subject in (b) is according to the A/B status comparison.
B45. The method of any one of embodiments B1-B44, wherein the test sample comprises cell-free DNA (cfDNA).
B46. The method of any one of embodiments B1-B44, wherein the test sample comprises circulating tumor DNA (ctDNA).
B47. The method of any one of embodiments B1-B46, wherein the test sample comprises blood plasma.
B48. The method of any one of embodiments B1-B46, wherein the test sample comprises urine.
B49. The method of any one of embodiments B1-B46, wherein the test sample comprises cerebrospinal fluid.
B50. The method of any one of embodiments B1-B49, wherein the population of cells in (b) comprises diseased cells.
B51. The method of any one of embodiments B1-B50, wherein the population of cells in (b) comprises cancer cells.
B52. The method of any one of embodiments B1-B50, wherein the population of cells in (b) comprises diseased kidney cells.
B55. The method of any one of embodiments B1-B50, wherein the population of cells in (b) comprises diseased bladder cells.
B56. The method of any one of embodiments B1-B50, wherein the population of cells in (b) comprises diseased neuronal cells.
B57. The method of any one of embodiments B1-B56, further comprising detecting a presence or absence of a disease or a disease subtype for the subject according to the presence or absence of the population of cells detected in (b).
B58. The method of embodiment B57, wherein the disease is cancer.
B59. The method of embodiment B58, wherein the disease is MDS.
B60. The method embodiment B59, wherein the disease is MDS-RS-MLD.
B61. The method of embodiment B57, wherein the disease is kidney disease.
B62. The method of embodiment B57, wherein the disease is bladder disease.
B63. The method of embodiment B57, wherein the disease is a neurodegenerative disease.
B64. The method of any one of embodiments B1-B63, comprising any of the features, or parts thereof, of embodiments A1-A67.

### EXAMPLES

The examples set forth below illustrate certain implementations and do not limit the technology.

### Example 1: Fragment Length Analysis

This Example shows that the ss prep method described herein, in combination with an enrichment step, can be used to profile and quantify tumor-derived DNA within cfDNA, and distinguish myelodysplastic syndrome (MDS) groups and acute myeloid leukemia (AML) groups from healthy samples based on fragment size ratio.

DNA sequencing library preparation is a crucial step for next-generation sequencing (NGS) of cell free DNA of plasma for cancer diagnostics. Described in this Example is a robust single-stranded DNA library preparation method that generates libraries with unique molecule identifiers (UMIs), with advantages over traditional double-stranded DNA preparations. Using 5 ng of plasma-derived cfDNA from subjects with acute myeloid leukemia (AML) and myelodysplastic syndrome (MDS), both single-stranded (ssDNA) and double-stranded DNA (dsDNA) sequencing libraries with UMIs were generated. The libraries were enriched using a Twist Biosciences custom hybridization probe set for an 800 kilobase cancer mutation panel. The libraries were sequenced to a panel depth of 1000× to 2000×, after correcting UMI sequences and removing duplicate reads. With comparable fold enrichment and on-target percentages, complexities of 11-21 million unique molecules were observed with ssDNA libraries. This is approximately four times higher than dsDNA complexity of 2.8-4.9 million unique molecules. Using duplicate reads from the same unique molecule, error correction was performed on template read bases and low-variant allele frequency mutations were called. In particular, an increase in KRAS p.G12D variant allele fraction during progression from MDS to AML was shown. The ssDNA prep showed increased recovery of small fragments compared to dsDNA preparations and retained the native lengths and termini of sample fragments. Apart from improved library complexity, MDS and AML samples were distinguished from healthy samples by their disrupted genomic profile of short to long fragments.

Sample Procurement and DNA Preparation

Plasma was obtained from patients that were diagnosed with MDS or related AML at the Columbia University Medical Center under IRB protocol. The plasma was prepared according to a standard operating protocol. Briefly, whole blood was collected in 10 ml Streck Blood Collection tube and immediately spun at 1800 G for 10 minutes at 4° C. to separate plasma. The plasma was cleared of debris with a second spin at 16000 G for 10 minutes at 4° C. The cleared plasma was frozen in 2 ml aliquots. The frozen plasma was thawed at 37° C. for 5 minutes and spun at 12000 G for 10 minutes to remove cryoprecipitates. cfDNA was extracted from 2 ml of thawed plasma using the QIAGEN QIAAMP MINELUTE ccfDNA kit following the manufacturer's instructions and eluted in 60 μl water.

NGS Library Preparation and Exome Panel Enrichment

Libraries were generated with 5 ng of cfDNA using either Claret Bioscience's SRSLY™ PicoPlus Library Prep Kit (ss prep) and manufacturer's instructions specific for the low fragment retention protocol or NEBNext Ultra II Kit (ds prep). Both protocols were performed with the addition of Unique Molecular Identifiers (UMI). Post library construction, libraries were pooled together and enriched using Twist Bioscience's Custom Tumor Mutation Panel and manufacturer's instructions. Enriched libraries were then sequenced to a depth of greater than 1000× on an Illumina® HiSeq X at a read length of 2×151 bp following manufacturer's instructions.

Informatic Processing

Sequence data was converted to FASTQ with bcl2fastq v 2.20.0.422. Libraries were trimmed with SeqPrep2. Reads were aligned to the hg19 reference with BWA v0.7.15-r1140 with sampe and samse for paired and merged reads, respectively. Duplicates were marked and removed with GATK version 2.17.1. UMI deconvolution was performed using SRSLYumi, a Python package developed by ClaretBio (claretbio.com/products/software). Complexity analysis was performed using Preseq or PicardTools (GATK v2.171). Variant regions within the 800 kb panel was downloaded from the Twist Bioscience website, the ensemble variant caller with two passes including VarDict, Mutect2, and Strelka2. All other parameters were set to the defaults.

Results

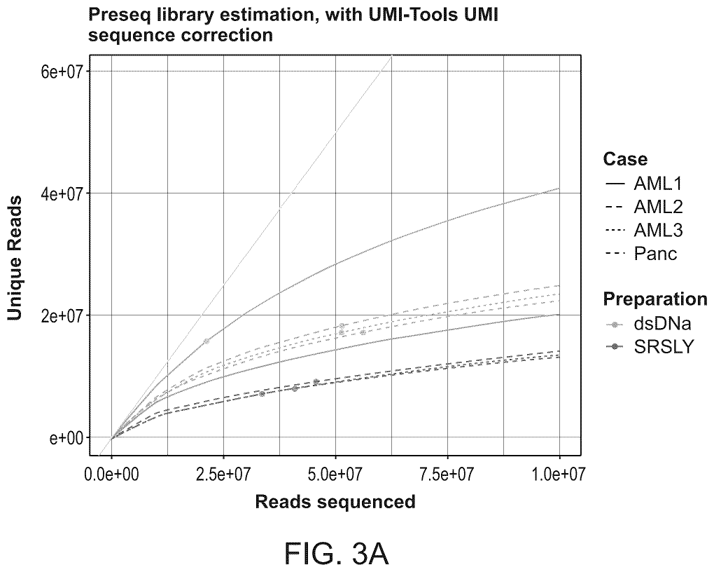

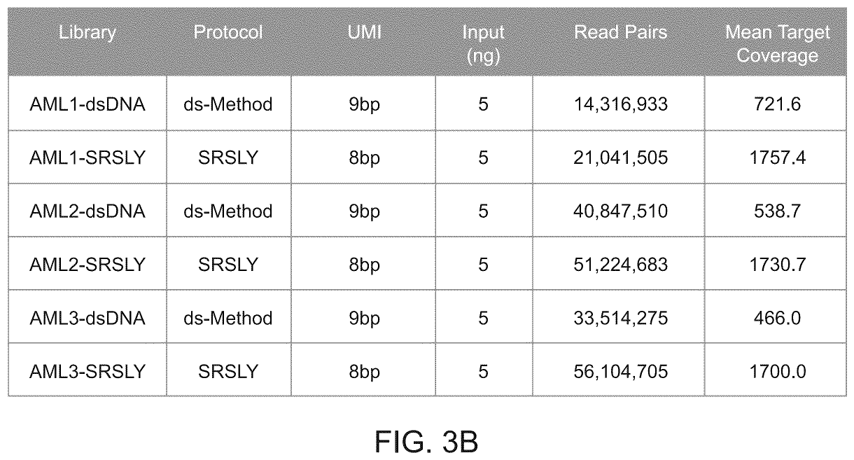

It was observed that libraries generated using Claret Bioscience's SRSLY™ Library Prep Kit (ss prep) had higher complexity and mean target coverage compared to the libraries generated using the NEBNext Ultra II Kit (ds prep) (FIGS. 3A and 3B). This suggests that high-quality data can be generated from lower input of cfDNA using the ss prep method described herein in comparison to double-stranded methods.

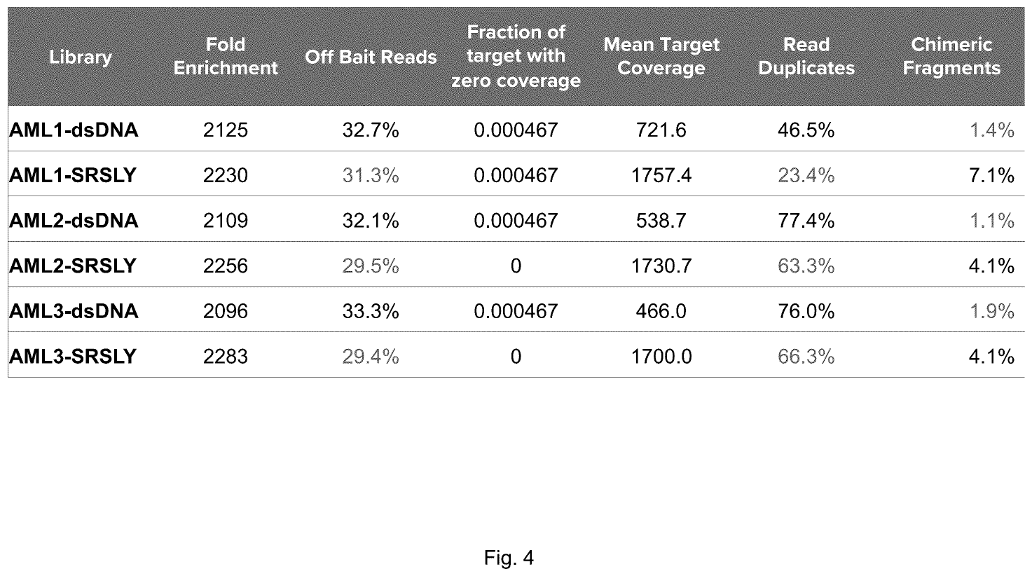

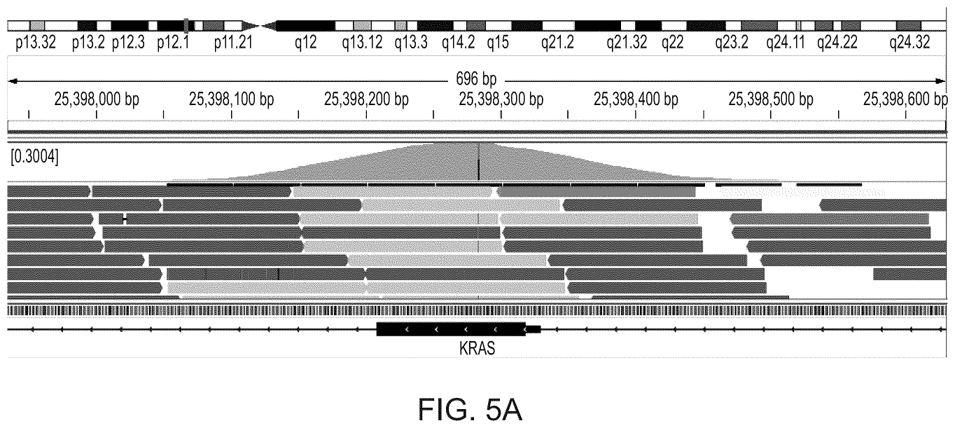

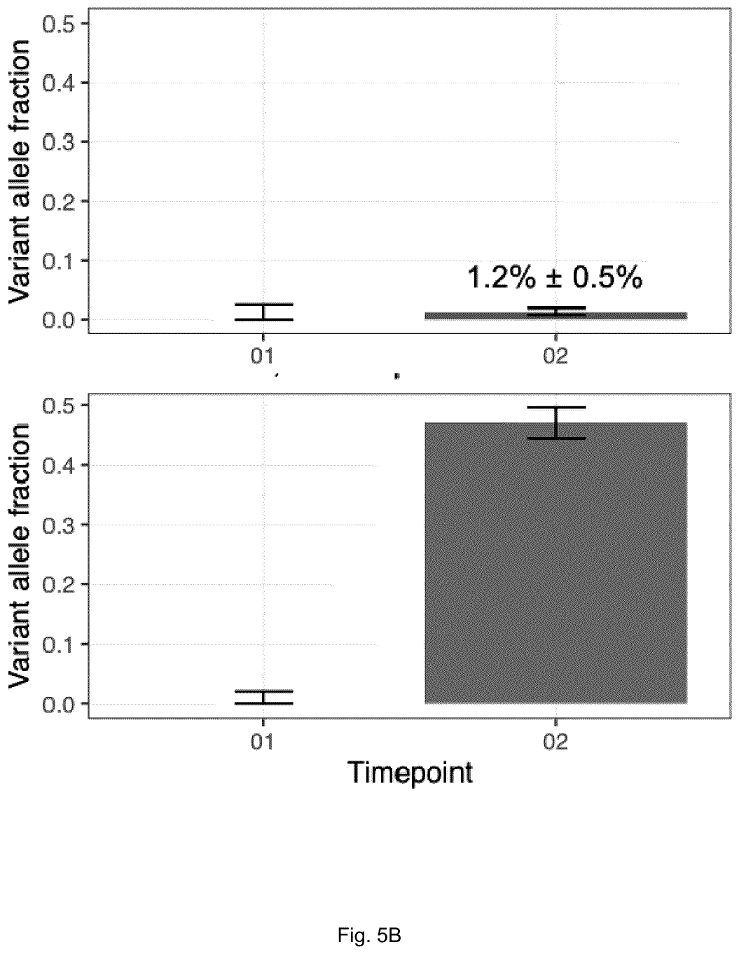

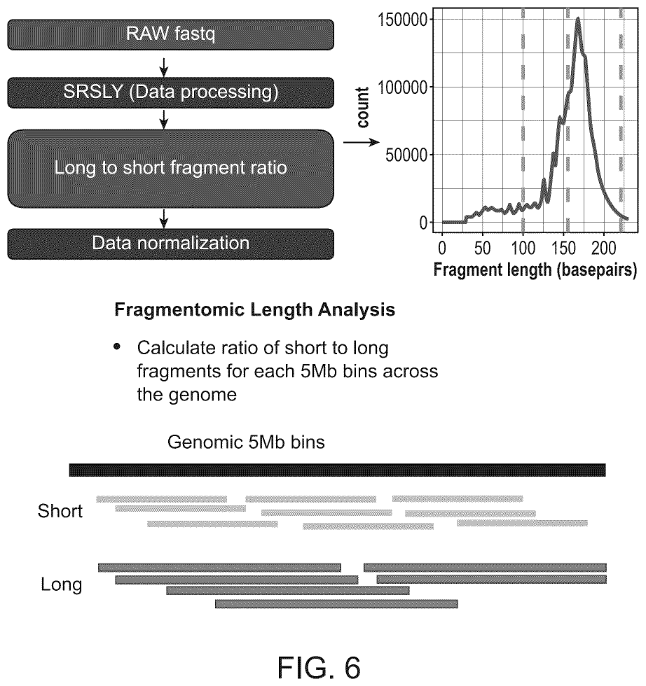

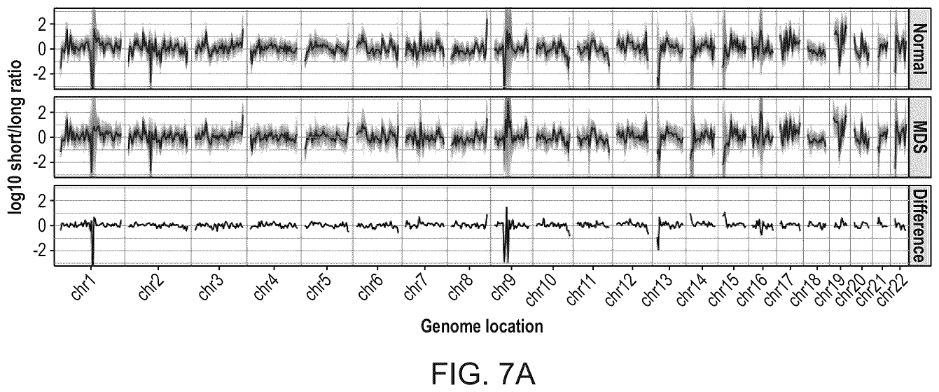

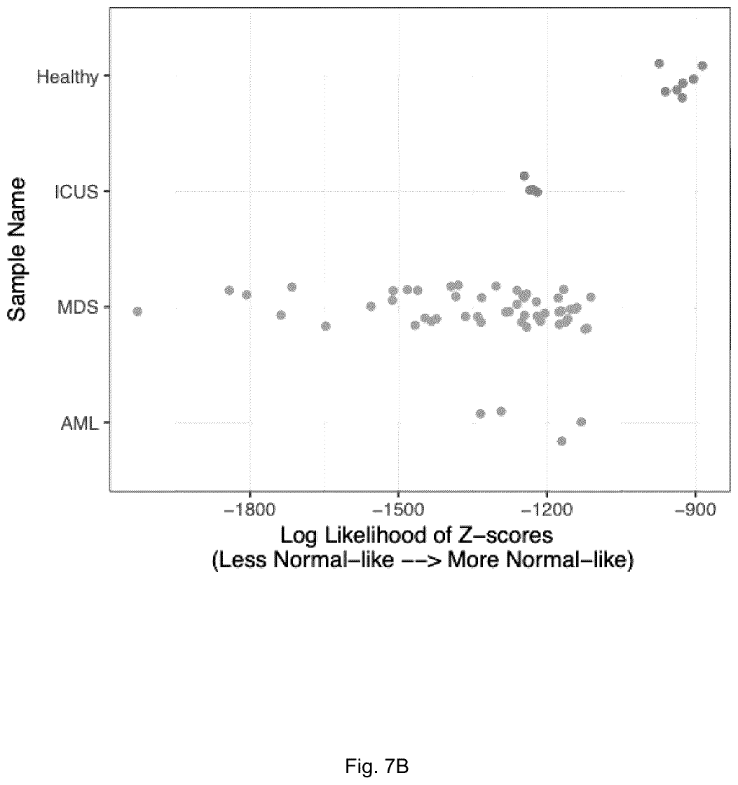

Evaluation of other general enrichment metrics showed that libraries generated using Claret Bioscience's SRSLY™ Library Prep Kit (ss prep) have improved fold enrichment, slightly lower off-bait reads, lower duplicate reads (FIG. 4). Overall, due to improved library complexity, the specificity of the downstream enrichment is improved in the ss prep libraries.

Using ClaretBio's customized package SRSLYumi, the reads were deconvoluted, read-deduplication was performed, and unique library molecules were identified. Following error correction, low allele-fractions were identified. Changes in variant allele frequency (VAF) in a KRAS G12D variant were detected among longitudinal samples where one time point had AML and the other had MDS. This shows that Claret Bioscience's SRSLY™ Library Prep Kit (ss prep) can be used in traditional mutational calling analysis.

In certain applications, cfDNA fragment length can be informative in cancer prediction. Tumor-derived DNA generally is shorter than DNA derived from normal tissue. In certain applications, long to short fragment ratio can be used to predict cancer in individuals with cancer (e.g., lung cancer).

Using a similar approach with the pilot cohort, this Example shows that cfDNA libraries generated using Claret Bioscience's SRSLY™ Library Prep Kit (ss prep) may be used to distinguish between individuals with MDS and healthy individuals. Across the genome, unique fragment length ratio profiles can be observed in individuals with MDS compared to those without. Using a Log-likelihood calculation, it was observed that an average profile for MDS using this approach is distinct from healthy. With a larger cohort, more nuanced signals may be captured with the various sub-groups of MDS and subsequently from AML.

### Example 2: Fragment Length Analysis

Chromatin is typically organized into two compartments labeled A and B. A/B compartment-associated regions generally are on the multi-Mb scale and correlate with either open and expression-active chromatin (A compartments) or closed and expression-inactive chromatin (B compartments). A compartments often are gene-rich, have high GC-content, contain histone markers for active transcription, are typically made up of self-interacting domains, and contain early replication origins. B compartments typically are gene-poor, compact, contain histone markers for gene silencing, are typically made up of lamina-associating domains (LADs), and contain late replication origins. In certain instances, A/B compartments may be refined into sub-compartments. In this Example, correlations between fragment length ratio, eigenvalues for genomic portions, and distance to boundary are shown, and the use of fragment length ratios to identify A/B transitions is demonstrated.

Defining Compartments

AB compartment data was taken from “Reconstructing A/B compartments as revealed by Hi-C using long-range correlations in epigenetic data”, Jean-Philippe Fortin & Kasper D. Hansen, Genome Biology volume 16, Article number: 180 (2015), which is incorporated by reference in its entirety.

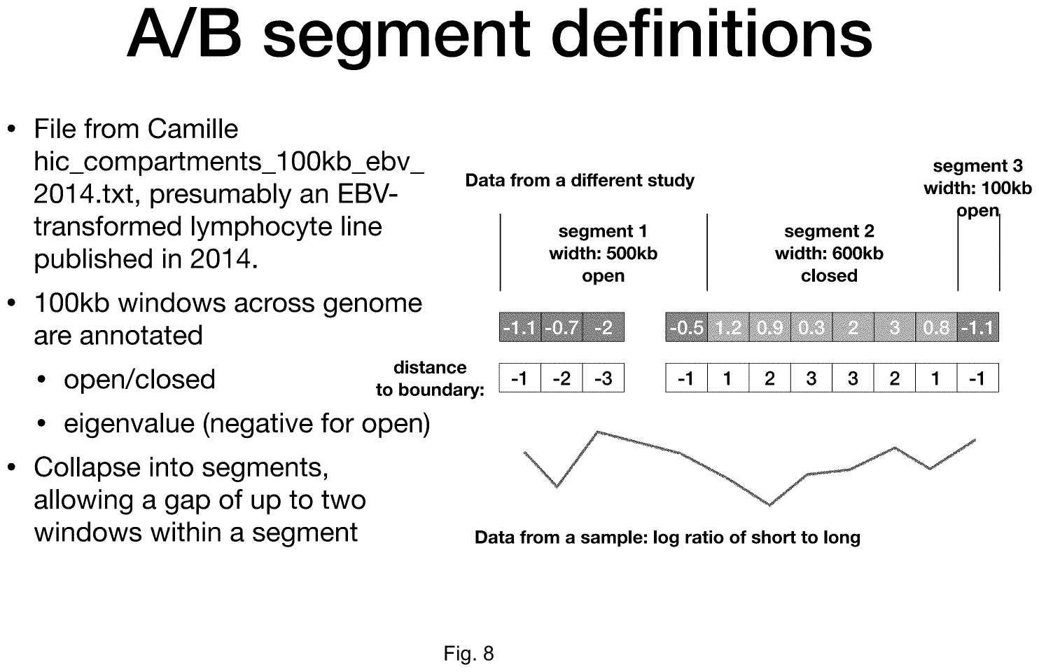

There are multiple gaps in the HiC A/B compartment definitions, where 100 kb windows disappear, and there are even more gaps in the ratio calculations. Accordingly, the compartments in this Example are based on the HiC data, allowing for a gap of up to 2×-100 kb windows (see FIG. 8).

There are a few outlier segments, with the largest being 35 MB, but the vast majority of compartments are less than 5 MB large.

Eigenvalue Versus Fragment Ratio

Three sets of fragment lengths are defined as follows:
1) small—0-99 bp
2) medium—100-149 bp
3) long/large—150-220 bp

A large dependency was observed between negative values and fragment ratios that increased with the small versus long fragment ratios.

Combined Samples

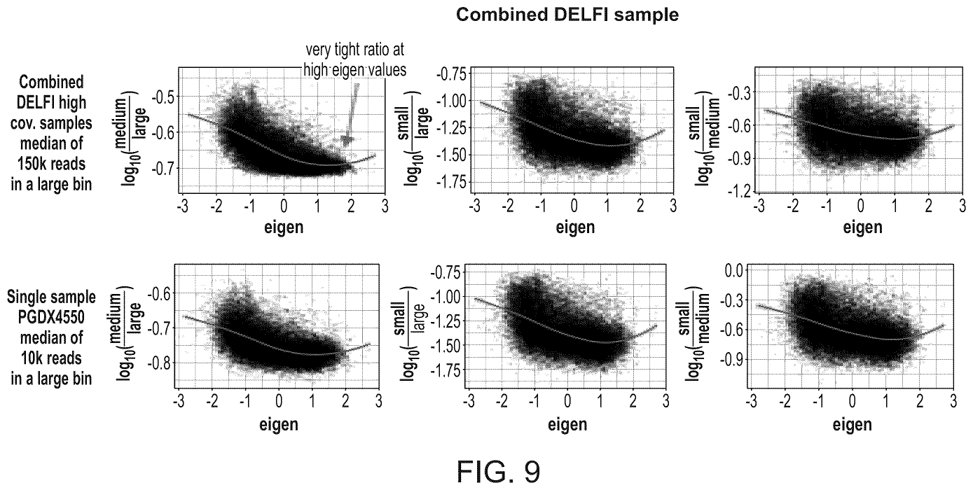

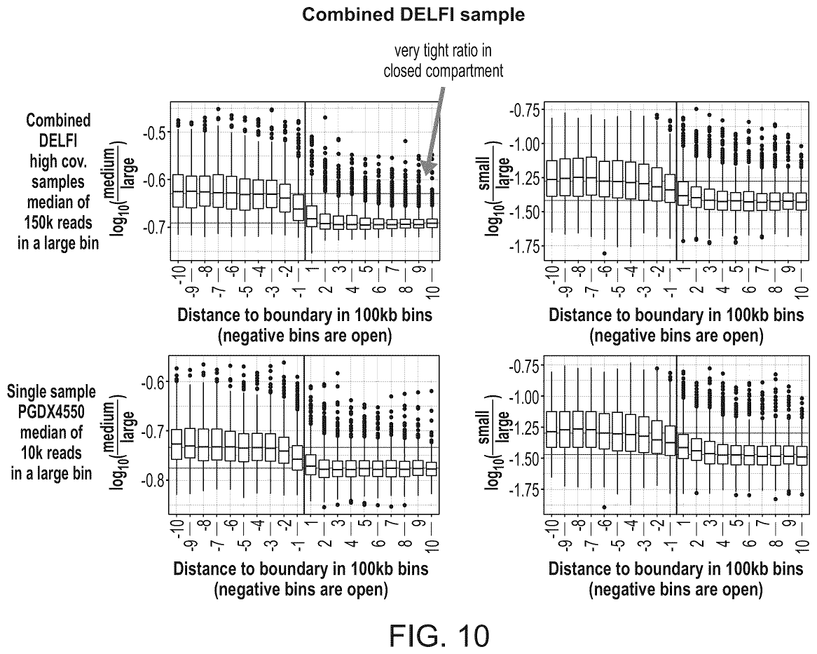

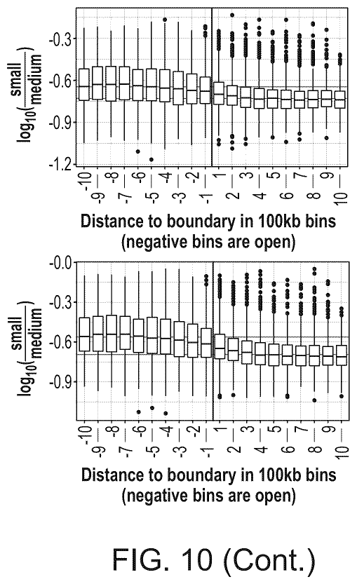

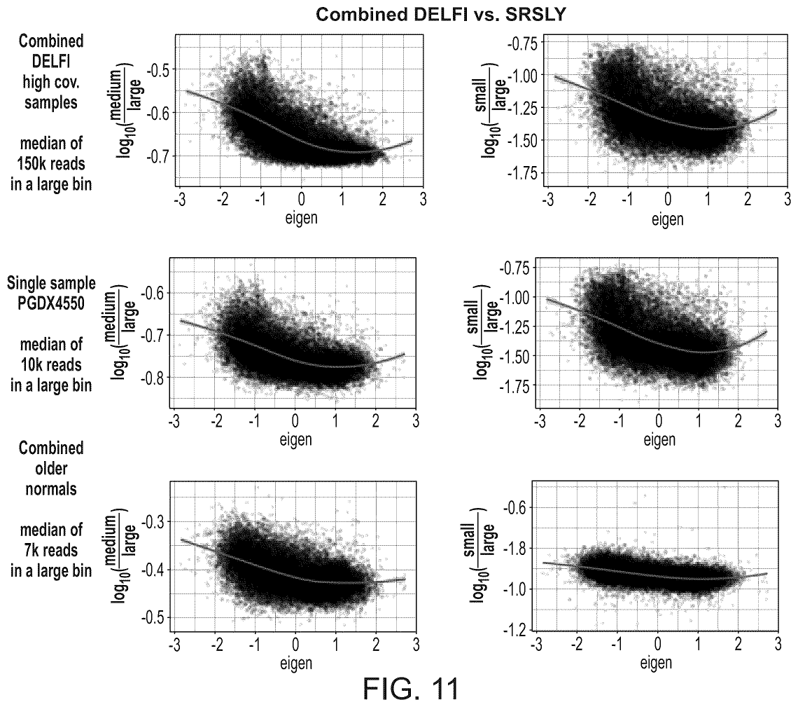

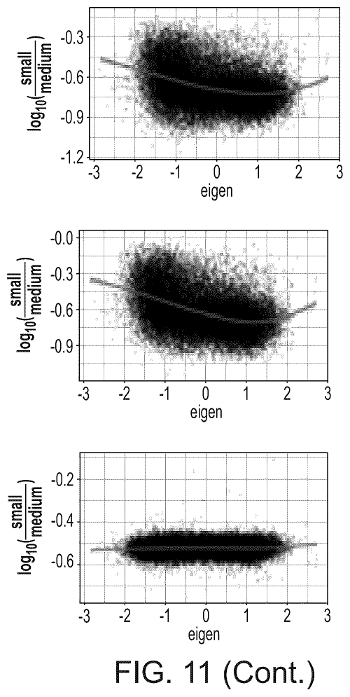

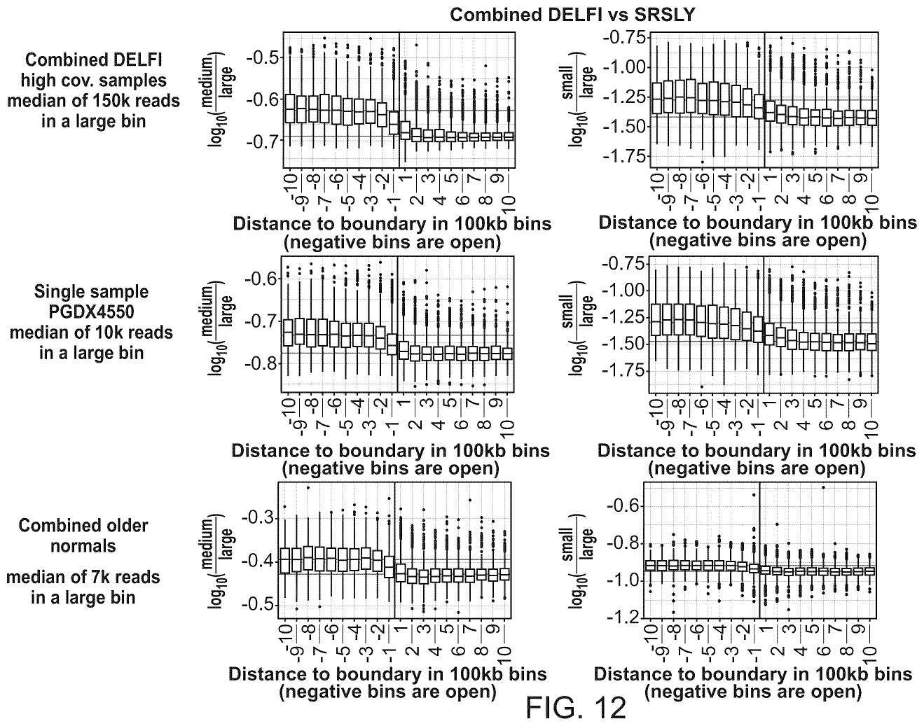

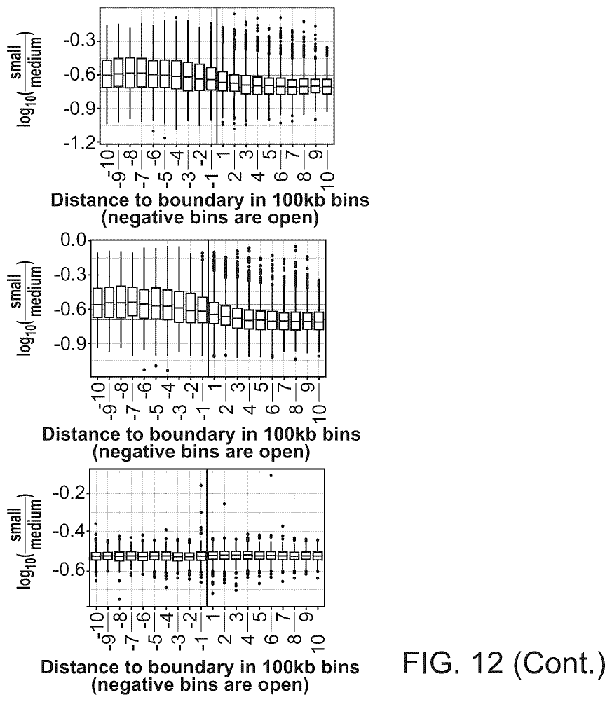

Samples were combined by adding their counts at each genomic portion (100 kb bin) and each fragment length bin. Combining ˜20 DELFI samples and ˜20 samples prepared using a single-stranded library prep method described herein (SRSLY), the DELFI combined had large fragment length bins with roughly ˜150,000 fragments, whereas the combined SRSLY sample had only about 7,000 fragments, fewer than a single DELFI high-coverage sample. However, this revealed strong trends (see FIGS. 9-12). The small fragment counts were reliable with the SRSLY samples, in that it was known they were small fragments. These small fragments show some compartment effect with the small/large ratio, but show no effect with small/medium. This indicates small/medium may be binned together for greater power when working with SRSLY samples.

MDS Samples

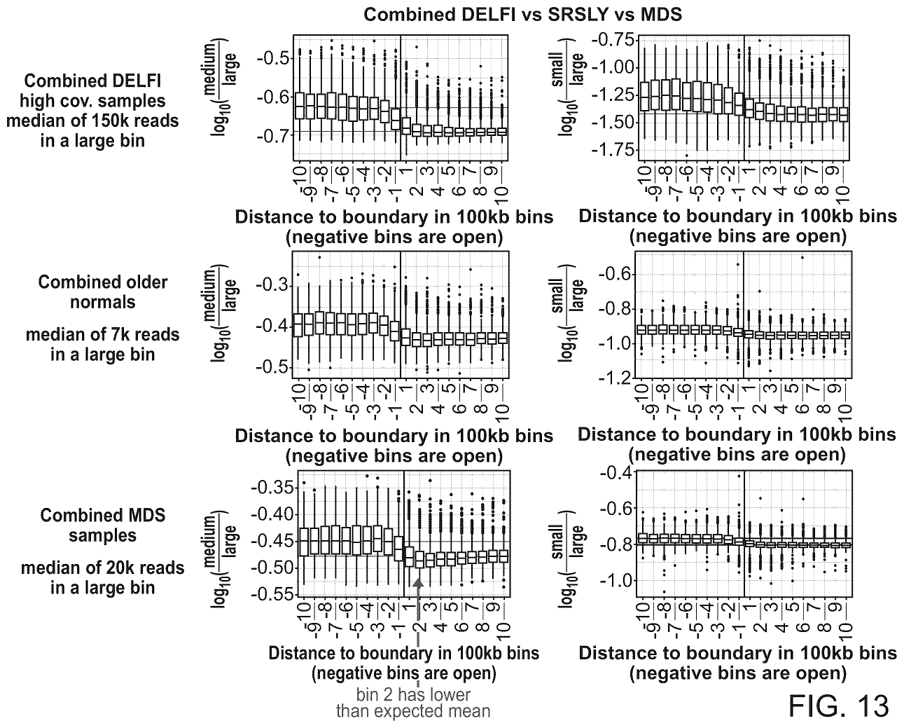

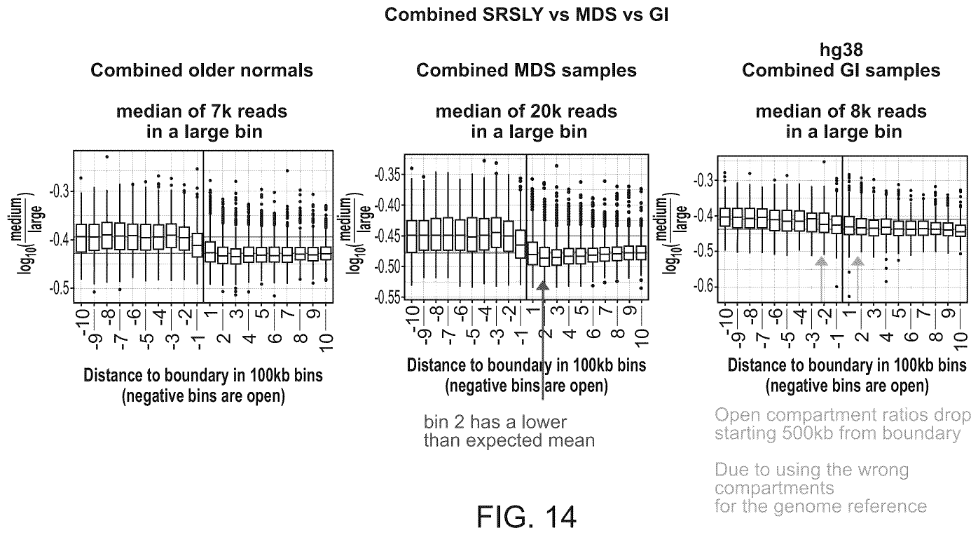

When looking at combined MDS samples, bin 2 in the closed compartment showed a strong drop of medium/large fragment ratio (FIGS. 13 and 14). This may be useful in distinguishing MDS from other conditions. Similarly there was a bit of a drop at bin −2, which is significant but with less overall effect.

Assessing Individual Compartment Transitions

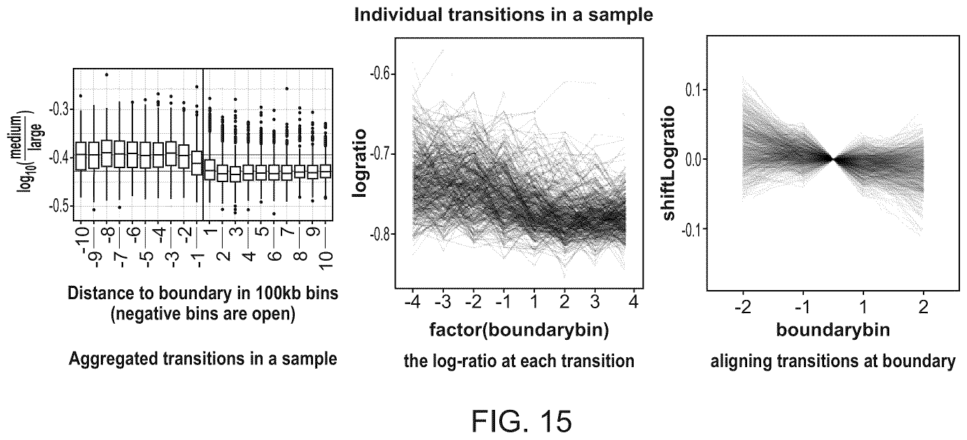

The boxplots shown in the figures for this Example aggregate bins across different transition points, which can obscure the trend for individual transitions. Individual transitions may be observed with lines (see FIG. 15). The third plot (from left to right) in FIG. 15 shows all the transitions shifted to cross 0 at the boundary. This shows that not all transitions for a single sample have a negative slope. Metrics for a compartment transition within a sample may include:
1) diff—difference of log ratio at bin 1 and bin −1;
2) diff2—difference of sum bin-1 and bin-2 minus sum of bin1 and bin2;
3) fit of the log ratio numbers to a linear regression that includes the eigen values of the bins and the mean values of the log ratios in other samples; and
4) fit of the log ratios and end termini to a logistic regression across many samples and sites using the bin eigen values, fragment log ratios within termini groups.

When using diff2, about 80% of the transitions in an example DELFI sample have a negative value for this. 0 is used as an initial cutoff for a significant downshift at the transition site (see FIG. 16).

Assessing Transition Scores Across Multiple Samples

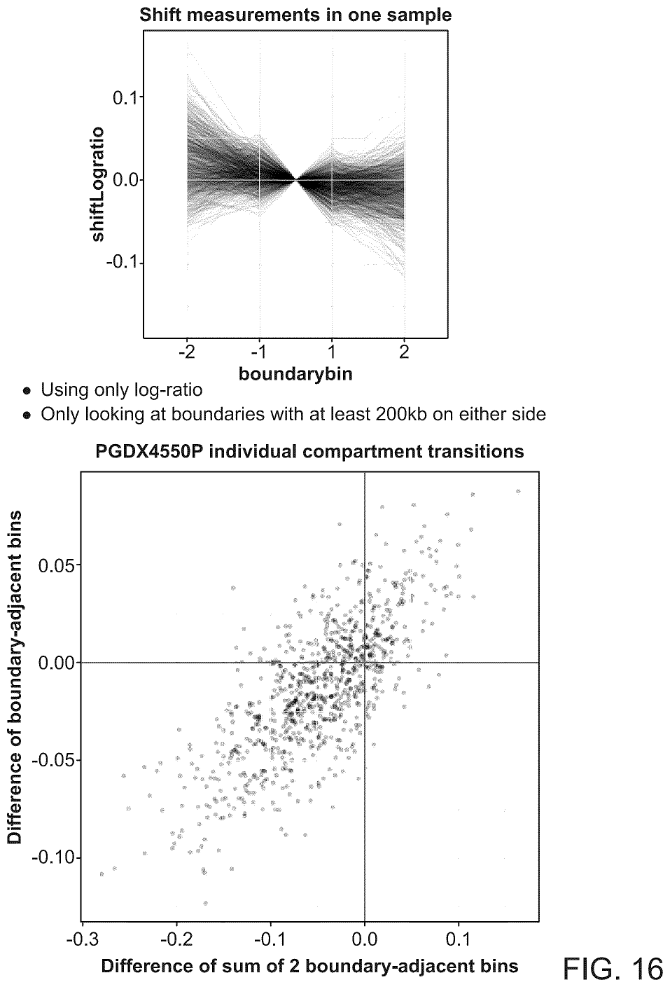

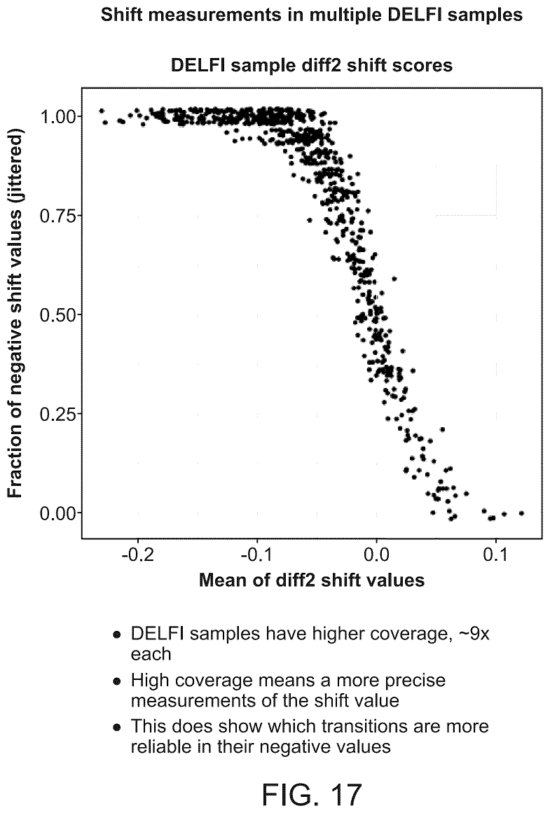

The DELFI set is the most deeply sequenced set of samples. Deeper sequencing of a sample typically provides a more precise measurement of the log ratios. Therefore, consistent compartment transition scores were observed with this set of samples. The transition scores are shown in FIG. 17. Each point is one genomic location of a compartment transition. The x-axis shows the mean transition score of the DELFI samples for that transition. The y-axis is the fraction of transition scores that are negative, meaning a drop in fragment ratio at the transition to closed. Conversely, there are several transitions that consistently increase the fragment ratio in the transition to closed, across all the DELFI samples.

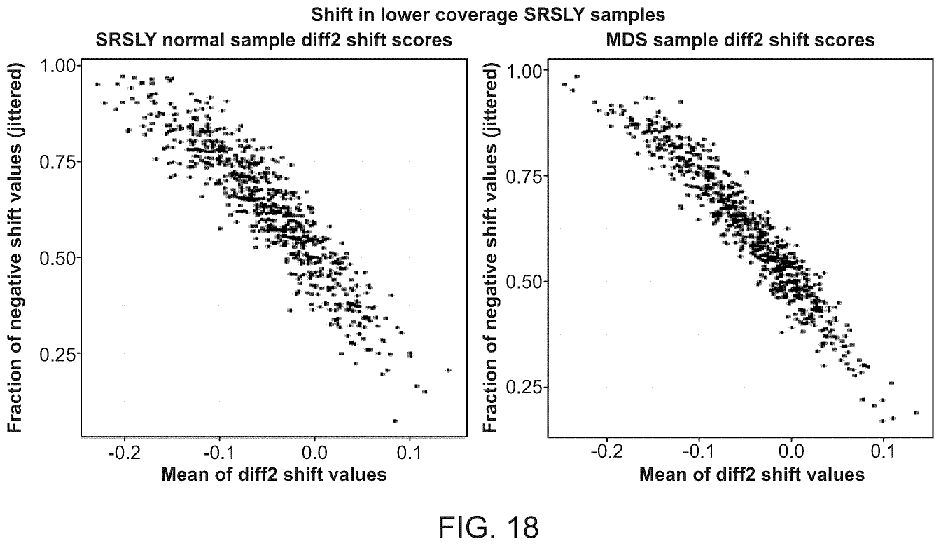

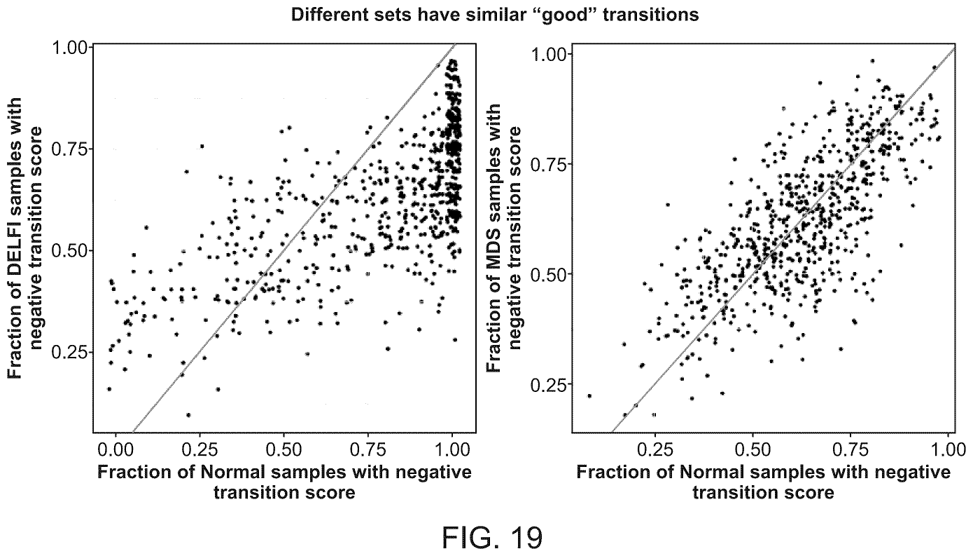

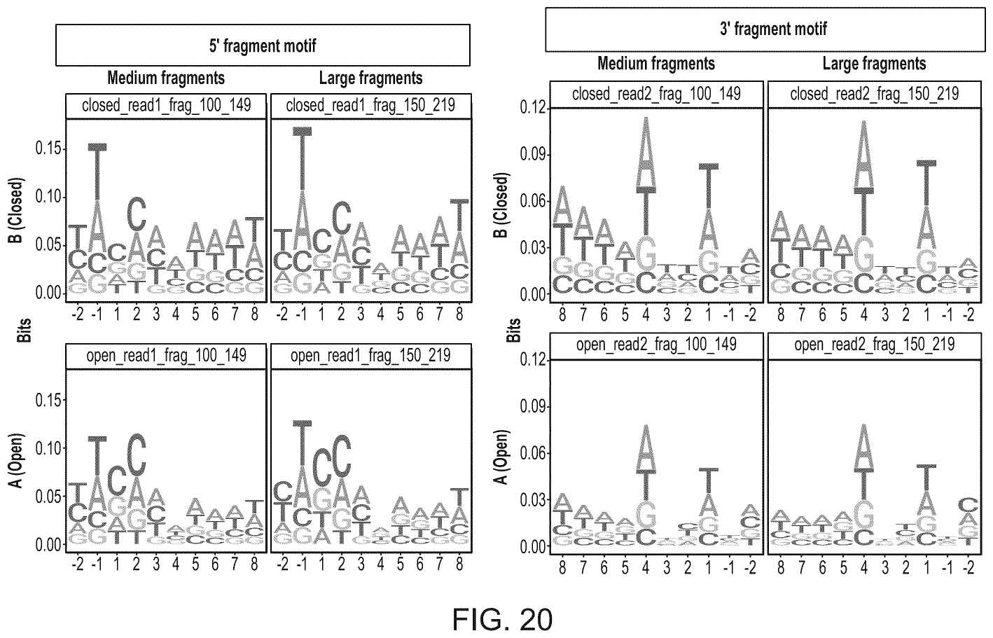

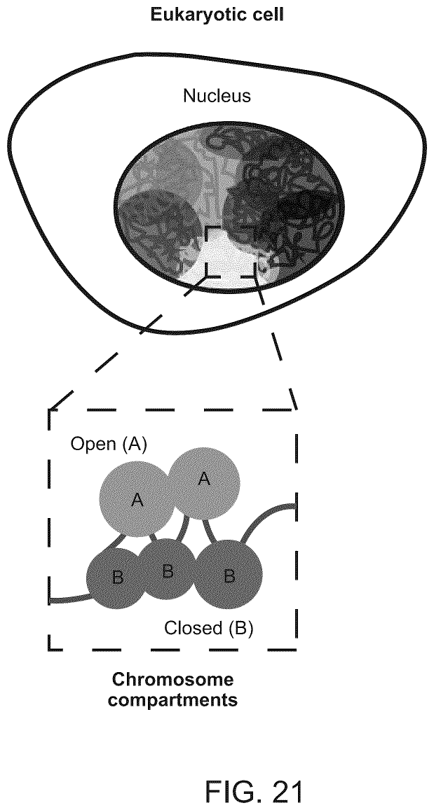

The normal and MDS sets (prepared using a single-stranded library prep method described herein (SRSLY)) were sequenced with lower coverage, so each individual sample's transition score had more noise. For the consistently negative diff2 transition points, this resulted in fewer of samples' transitions being negative. The MDS samples showed both a tighter overall concordance, and fewer constitutively negative transitions (see FIG. 18).

Conclusion

For dsDNA, 9× coverage, samples from the DELFI data, the eigenvalue of the open compartments correlated with fragment length ratios, with smaller fragment ratios having a larger correlation coefficient. The closed compartment eigenvalue varied less with the fragment ratio. Similarly, the distance of a bin to the compartment boundary was highly correlated to fragment length ratios. Further, the eigenvalue and boundary distance were generally complementary in their information, though most of the variance is covered by knowing only one of these.

The slopes of individual transitions vary, and do not all follow the overall trend that is seen in the aggregated boxplots.

The individual transitions deviate from the aggregated trend in a consistent manner that persists across different samples. This indicates a model may be developed for each transition, and each transition may be scored differently.

### Example 3: Fragment Length Analysis and MDS

In this Example, MDS samples and MDS subtype samples were subjected to a fragment length analysis.

Use of A/B Nuclear Compartment to Analyze Fragment Length

As described herein, the distance of a genomic window to its nearest closed-open transition may be used as a way to summarize fragment length ratios in various genomic windows. Using the A/B compartments of a GM12878 lymphoblastoid cell line (Rao et al., 2014 Cell 159(7):1665-80), a cfDNA sample from a cancer patient was found to have slightly different fragment length distributions compared to healthy in the open and closed compartment, which was visible with only 2.2 million read pairs of sequencing (approximately 0.1-fold coverage of the whole genome).

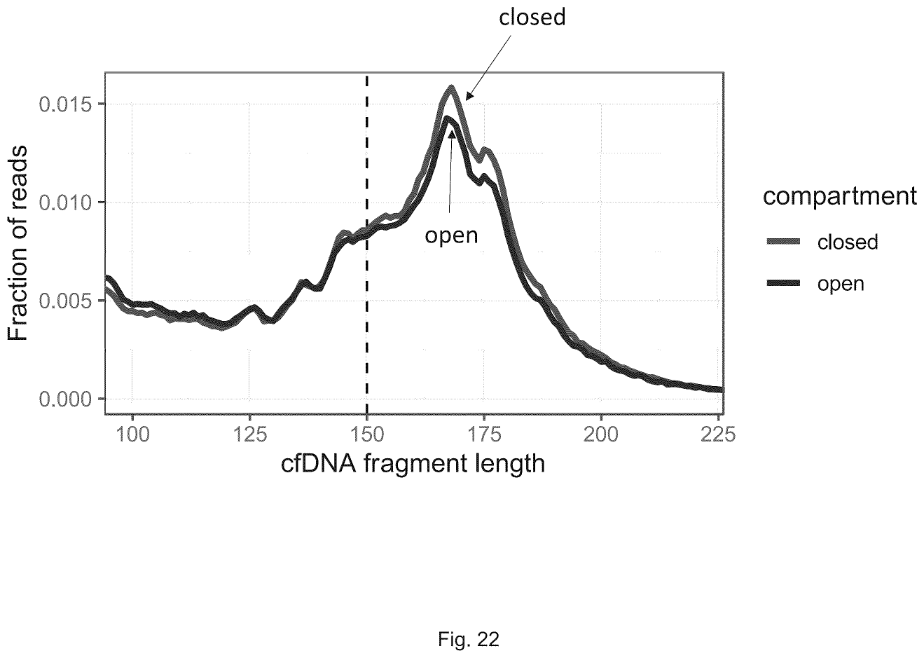

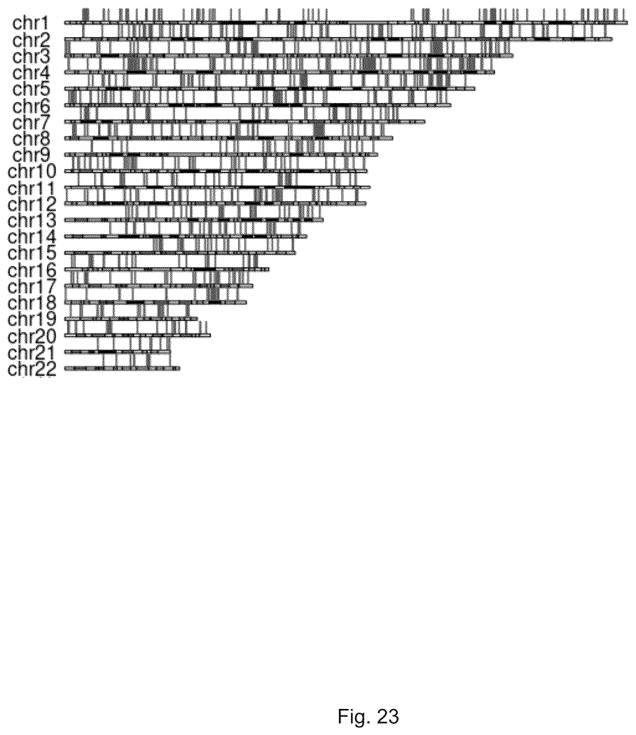

The difference shifts around 140-150 bp, and again at roughly 220 bp, with the closed compartment having more reads in the bin of 150 bp-220 bp than the open region (see e.g., FIG. 22, FIG. 25).

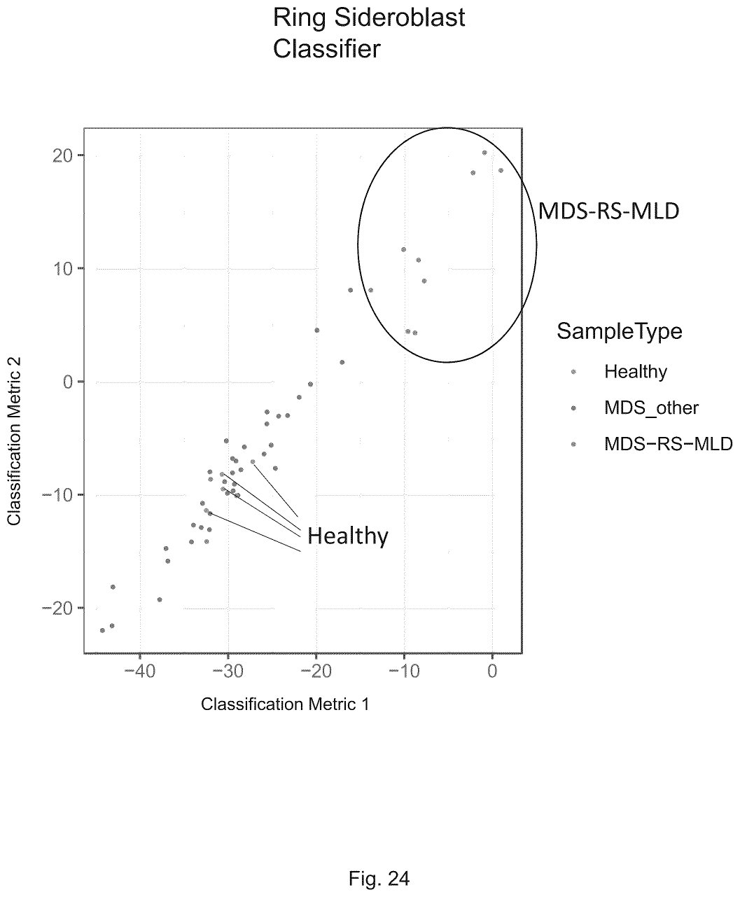

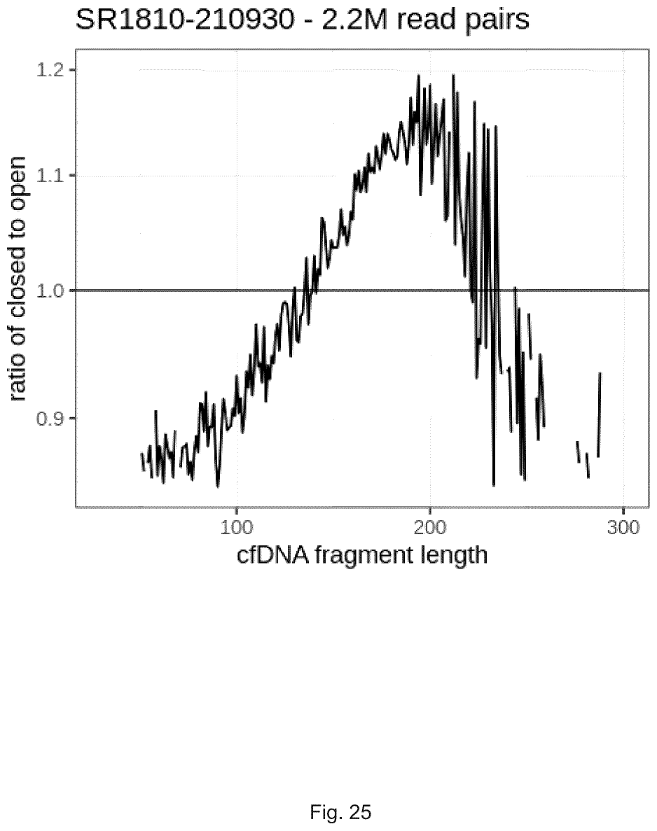

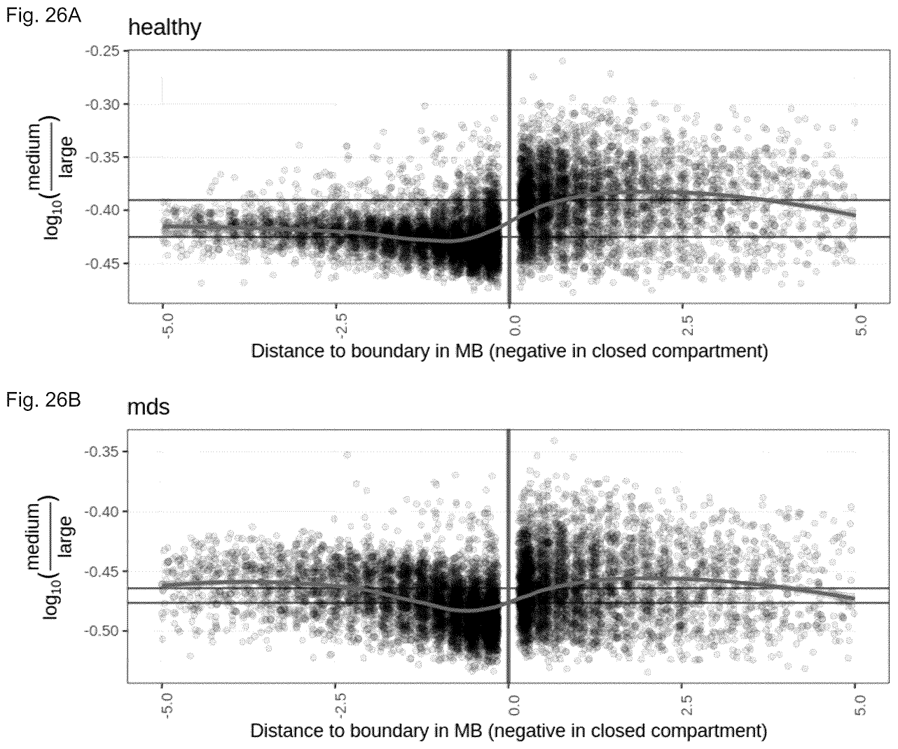

This view can be changed to pile up all of the genome along the various transitions from closed compartment to open compartment, as demonstrated in healthy tissues (FIG. 26A). For each 250 kb genomic window, the log₁₀ of the ratio of medium fragments (100 bp to 149 bp) to large fragments (150 bp to 220 bp) was calculated. These transitions may be filtered out to the maximally informative closed to open regions, for example by looking only at those transitions that differ between lymphoblastoid compartments and the compartments in another cell type of interest.

Deep Sequencing of MDS Dataset Reveals Changed Genomic Regions

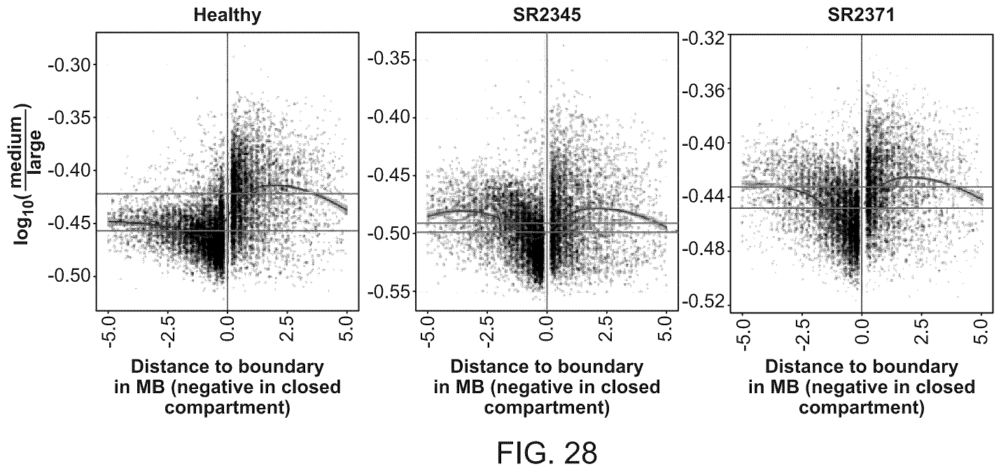

Several samples from a myelodysplastic syndrome (MDS) dataset, along with healthy samples, were subjected to deep sequencing (i.e., 15×-40× whole genome coverage). This allowed for much higher precision estimates of medium to long fragment ratio in 250 kb genomic windows. This revealed a large change in MDS samples versus healthy samples and other conditions (see FIG. 26B and FIG. 28).

Changed MDS Regions are Consistent in Two Deeply Sequenced Samples

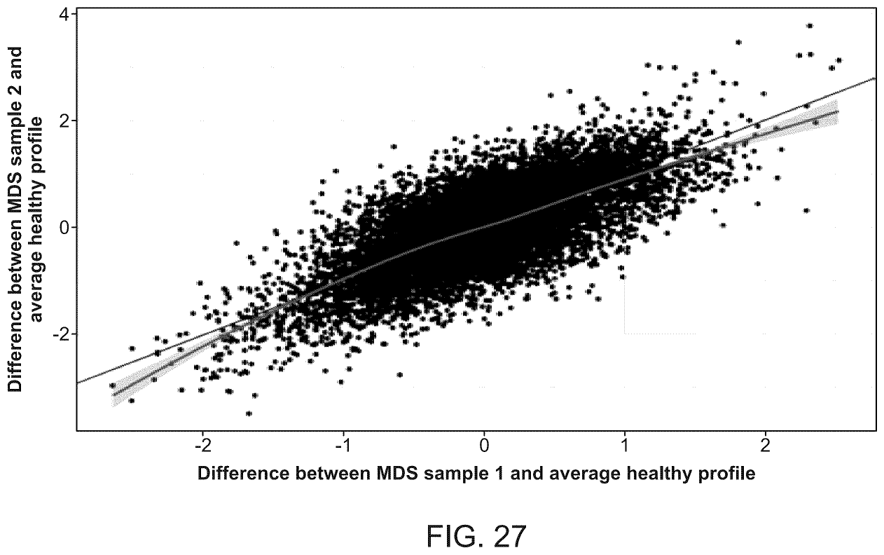

An average healthy fragment ratio profile was calculated from 10 deeply sequenced healthy samples, and two deeply sequenced MDS samples were compared to this, showing that the two MDS samples differed from the average healthy profile consistently across the genome (FIG. 27). The changed regions are referred to as the “opening” regions and generally are those with a difference above the 90th percentile or below the 10th percentile of all 250 kb regions.

Evaluation of Shallow 5M Sequencing of MDS Samples Reveals Association with RS-MLD

Samples were then subjected to very shallow sequencing (i.e., 5 million read pairs) to see if the same pattern was potentially visible. By visual inspection, the same “opening” phenomenon was observed in the original samples at 5 million read pairs (FIG. 28).

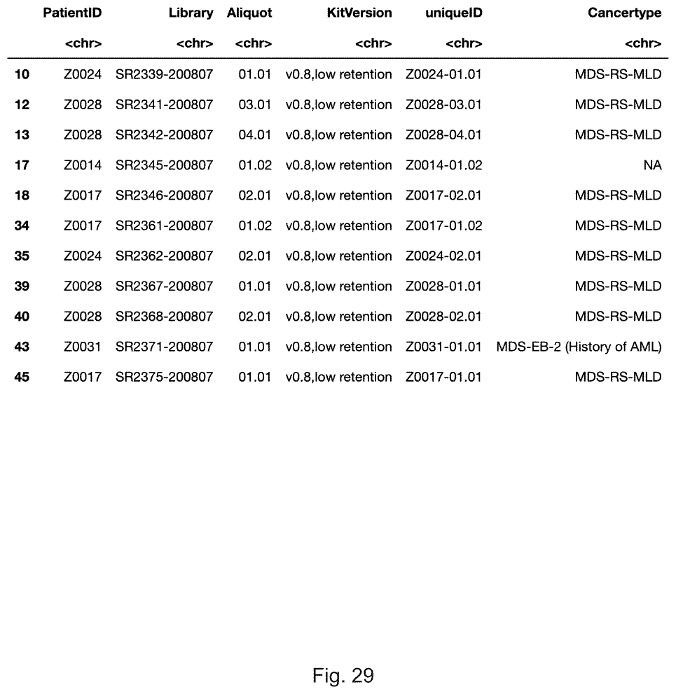

Visual inspection of all samples sequenced at 5 million read pair depth identified a list of samples with this “opening” of the closed compartment regions (FIG. 29). Most of the samples were of the MDS-RS-MLD subtype of myelodysplastic syndrome (MDS). This subtype is characterized by multiple lineage dysplasia (multiple types of blood cell are affected) and the presence of ring sideroblasts (early red blood cells in the bone marrow that have a ring of iron in them).

Machine Learning Classifier Separates Samples with Opened Lymphoblastoid-Closed Regions

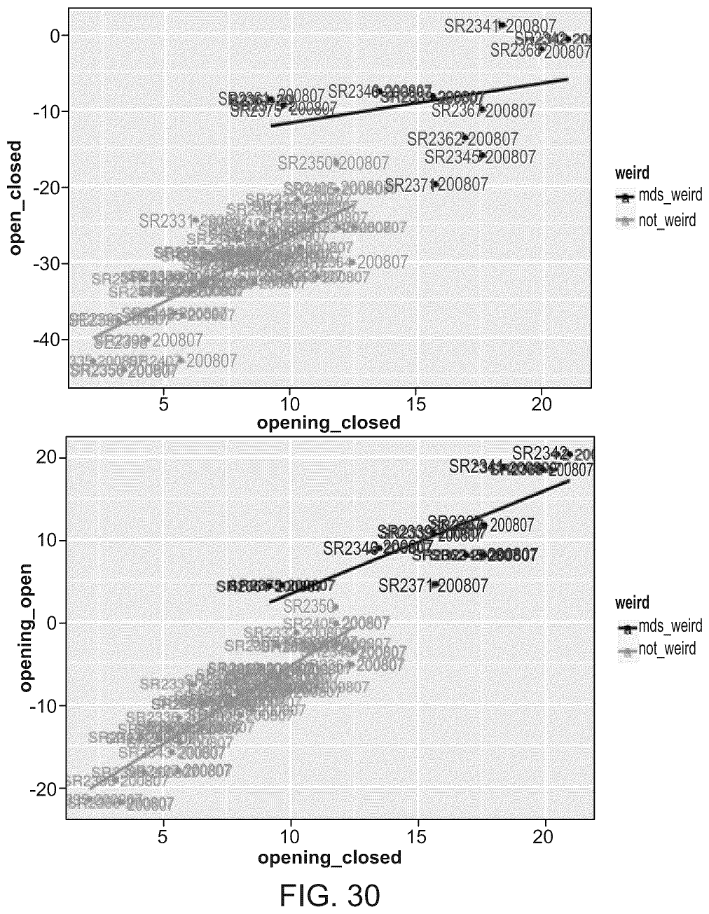

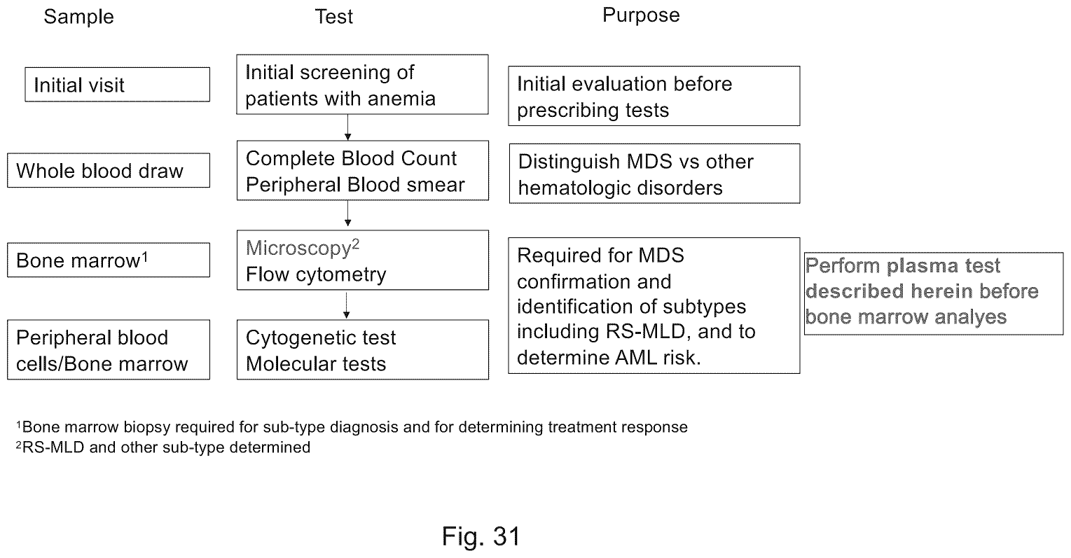

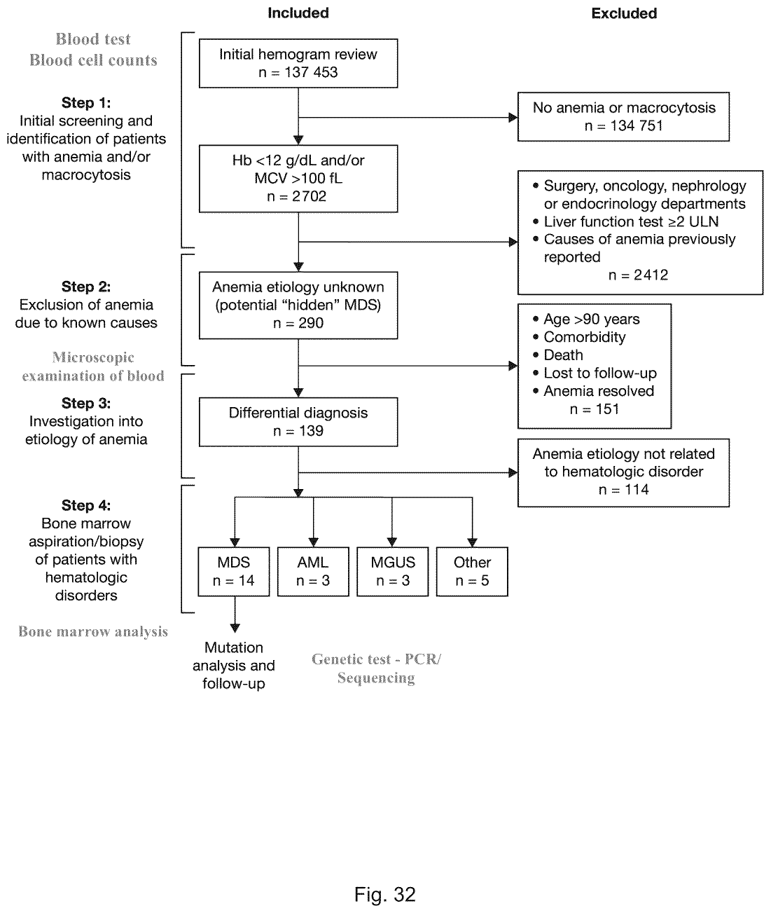

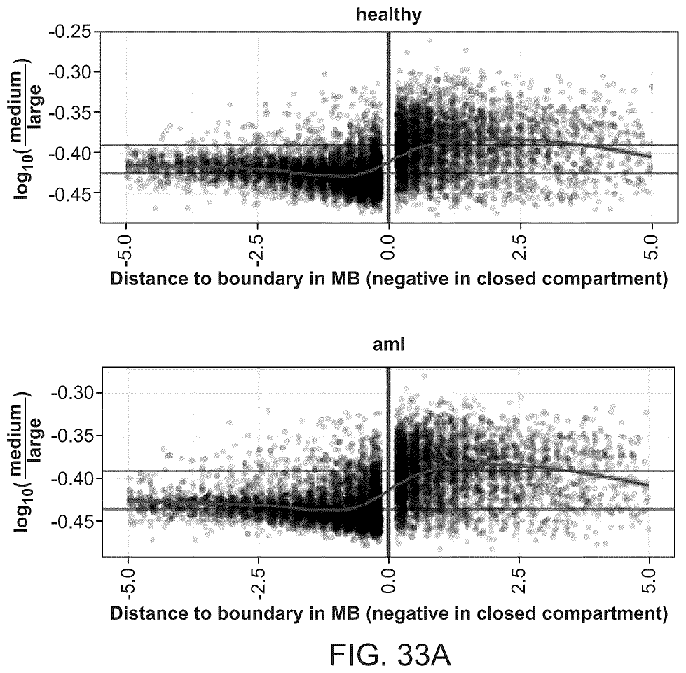

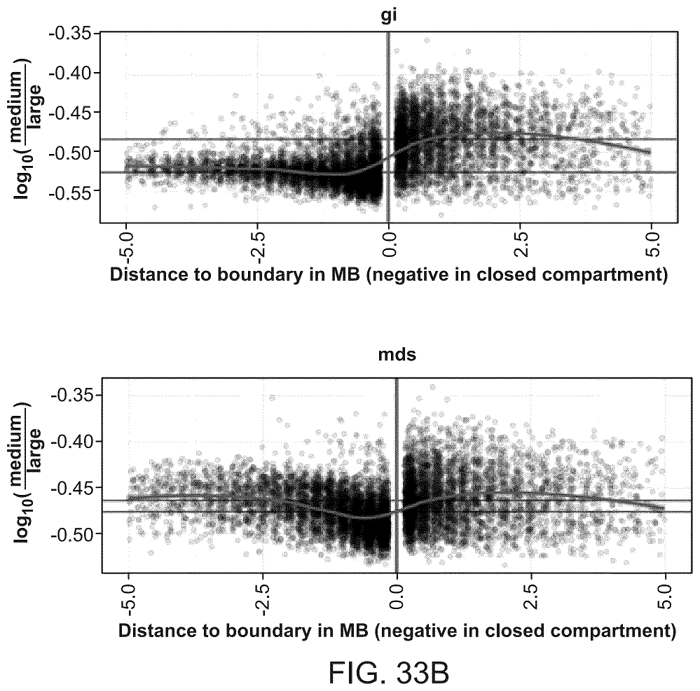

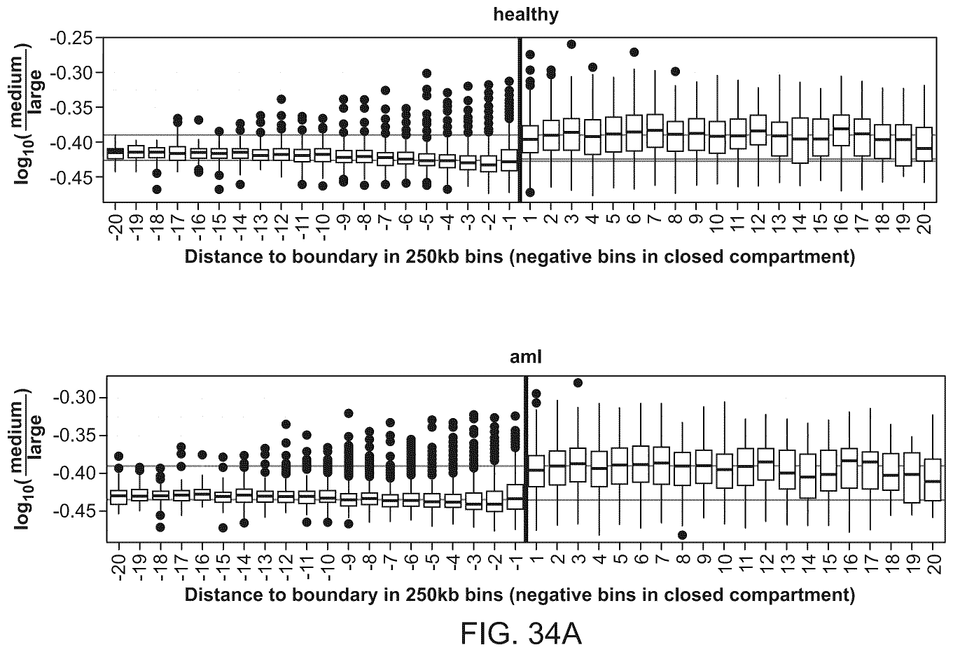

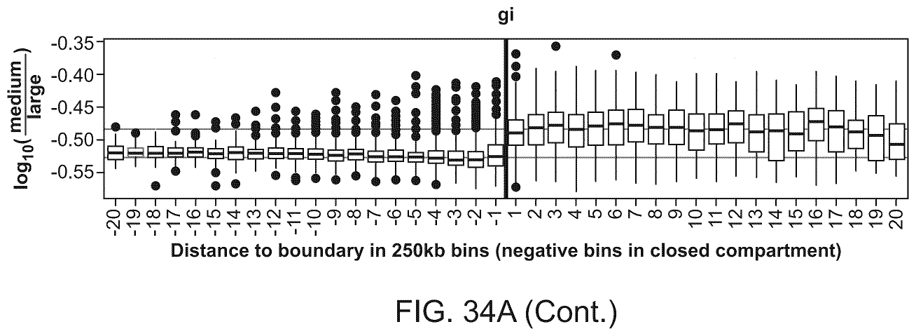

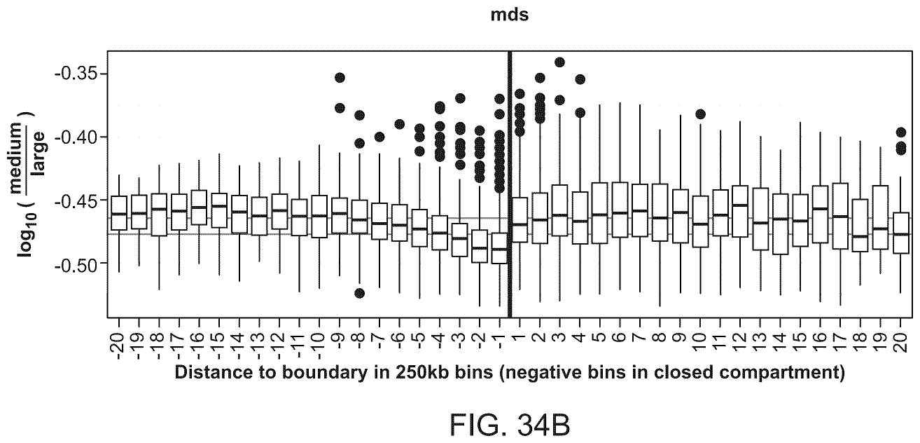

The genome was separated into three categories—open regions, closed regions, and the “opening” regions as described above. For each sample, t-tests were performed on the fragment ratio profile between all regions in each category, resulting in a set of three t-statistics per sample. These were then used to classify the samples into two categories—those that display an altered fragment ratio profile in the “opening” regions, and those that maintain a fragment ratio profile similar to that observed in healthy samples (FIG. 30). The samples labeled “mds_weird” are all the samples identified via visual inspection (FIG. 29).

### Example 4: Diagnostic/Research Device

In this Example, a device (e.g., a diagnostic device, a research device) is described that performs the following transformation:
Input: a biological sample that contains DNA or RNA that is fragmented according to the cell of origin of that nucleic acid. Examples of biological samples include blood plasma, urine, and cerebrospinal fluid.
Output: A set of potential cell compositions that are compatible with the fragments found in that sample.

Many such devices may be developed for different types of cell compositions. For example, a device may be developed to assess the content of Ring Sideroblast (RS) in a blood plasma sample for patients that have previously been diagnosed with Myelodysplastic Syndrome (MDS). In another example, a device may be developed to assess health of bladder or kidneys based on the composition of cell types that contribute to a urine sample. In another example, a device may be developed to evaluate the health of neural tissue in neurodegenerative patients from a cerebrospinal fluid sample.

The device uses a database of A/B compartment data as part of generating its output. This catalogs the open or closed status of every portion of the reference genome of the source organism of the sample, generated from one or more type of cells. This may include a single cell type, or an amalgamation of cell types, such as a Hi-C map generated from a collection of lymphocytes collected from a blood sample. This may contain an A/B map for every type of cell in a hematopoietic stem cell lineage tree, or A/B maps from whole tissues such as skeletal muscle, liver, or kidney, for example. This may also contain single-cell A/B maps of the cells that constitute tissues.

The design of the device and its applicability is determined by the design of the parameters below.

1. Selection of Regions to Analyze for Fragment Length

Depending on the application of the device, it may first restrict analysis to only particular regions of the genome that are informative for distinguishing between the tissues in the database. For example for a ring sideroblast device, it may select regions that differ between lymphoblastoid and RS A/B compartments, if an A/B map is available for both cell types. Selection also may be based on prior observations of fragment lengths, for example genomic windows where RS differs most from lymphoblastoid data (see Table 1 below for this example data). In addition to selection of regions that differ, there may be additional criteria for selection of subsets of the genome, such as consistent regions, that may be used as controls for the assessment in step 4.

TABLE 1

| contig | start | end | domain | contig | start | end | domain |
|---|---|---|---|---|---|---|---|
| chr1 | 8000001 | 8500000 | open | chr2 | 158000001 | 158250000 | closed |
| chr1 | 8750001 | 1.00E+07 | open | chr2 | 162500001 | 1.63E+08 | closed |
| chr1 | 10250001 | 10500000 | open | chr2 | 163750001 | 1.64E+08 | closed |
| chr1 | 17000001 | 17500000 | open | chr2 | 168500001 | 168750000 | open |
| chr1 | 19250001 | 19500000 | open | chr2 | 169000001 | 169250000 | closed |
| chr1 | 24250001 | 24500000 | open | chr2 | 173000001 | 173250000 | open |
| chr1 | 25000001 | 25250000 | open | chr2 | 174500001 | 174750000 | open |
| chr1 | 27000001 | 27250000 | open | chr2 | 184000001 | 184500000 | closed |
| chr1 | 27750001 | 28250000 | open | chr2 | 185000001 | 185750000 | closed |
| chr1 | 31750001 | 3.20E+07 | open | chr2 | 192500001 | 1.93E+08 | closed |
| chr1 | 35750001 | 36500000 | open | chr2 | 193250001 | 193500000 | closed |
| chr1 | 39250001 | 39500000 | open | chr2 | 197000001 | 197250000 | open |
| chr1 | 39750001 | 4.00E+07 | open | chr2 | 197750001 | 1.98E+08 | open |
| chr1 | 41500001 | 4.20E+07 | open | chr2 | 201250001 | 201500000 | open |
| chr1 | 42750001 | 4.30E+07 | open | chr2 | 207000001 | 207250000 | open |
| chr1 | 52500001 | 52750000 | open | chr2 | 207750001 | 2.08E+08 | open |
| chr1 | 56250001 | 56500000 | closed | chr2 | 211500001 | 212250000 | closed |
| chr1 | 58500001 | 5.90E+07 | open | chr2 | 213500001 | 2.14E+08 | closed |
| chr1 | 59500001 | 59750000 | closed | chr2 | 218000001 | 218250000 | open |
| chr1 | 61000001 | 61500000 | closed | chr2 | 224750001 | 2.25E+08 | open |
| chr1 | 64000001 | 64250000 | closed | chr2 | 230000001 | 230250000 | open |
| chr1 | 65250001 | 65500000 | closed | chr2 | 233250001 | 233500000 | open |
| chr1 | 66250001 | 66500000 | open | chr2 | 239000001 | 239500000 | open |
| chr1 | 73500001 | 73750000 | closed | chr20 | 1250001 | 1500000 | open |
| chr1 | 77250001 | 77500000 | closed | chr20 | 1750001 | 2.00E+06 | open |
| chr1 | 89000001 | 89250000 | open | chr20 | 4750001 | 5250000 | open |
| chr1 | 89250001 | 89500000 | closed | chr20 | 8250001 | 8500000 | closed |
| chr1 | 94500001 | 94750000 | closed | chr20 | 18500001 | 18750000 | open |
| chr1 | 95000001 | 95250000 | closed | chr20 | 19250001 | 19500000 | closed |
| chr1 | 105500001 | 105750000 | closed | chr20 | 19750001 | 2.00E+07 | closed |
| chr1 | 107750001 | 1.08E+08 | closed | chr20 | 20250001 | 20500000 | closed |
| chr1 | 109750001 | 1.10E+08 | open | chr20 | 20500001 | 20750000 | open |
| chr1 | 112250001 | 112500000 | open | chr20 | 32000001 | 32250000 | open |
| chr1 | 115750001 | 1.16E+08 | closed | chr20 | 40750001 | 4.10E+07 | closed |
| chr1 | 153250001 | 153500000 | open | chr20 | 45000001 | 45250000 | open |
| chr1 | 154250001 | 154500000 | open | chr20 | 47500001 | 47750000 | open |
| chr1 | 158500001 | 1.59E+08 | closed | chr20 | 48500001 | 4.90E+07 | open |
| chr1 | 159750001 | 1.60E+08 | open | chr20 | 50500001 | 50750000 | open |
| chr1 | 160500001 | 160750000 | open | chr20 | 53500001 | 5.40E+07 | open |
| chr1 | 161000001 | 161250000 | open | chr20 | 59750001 | 60250000 | closed |
| chr1 | 167500001 | 167750000 | open | chr20 | 62500001 | 62750000 | open |
| chr1 | 169500001 | 169750000 | open | chr21 | 14500001 | 14750000 | open |
| chr1 | 183500001 | 183750000 | open | chr21 | 21500001 | 21750000 | closed |
| chr1 | 185500001 | 185750000 | closed | chr21 | 29000001 | 29250000 | open |
| chr1 | 186500001 | 186750000 | closed | chr21 | 33000001 | 33500000 | open |
| chr1 | 188500001 | 1.89E+08 | closed | chr21 | 37750001 | 3.80E+07 | closed |
| chr1 | 189250001 | 190750000 | closed | chr21 | 38250001 | 38500000 | closed |
| chr1 | 192750001 | 1.93E+08 | closed | chr21 | 42250001 | 42500000 | open |
| chr1 | 195000001 | 195250000 | closed | chr21 | 45250001 | 45500000 | open |
| chr1 | 198500001 | 198750000 | closed | chr21 | 46250001 | 46500000 | open |
| chr1 | 200250001 | 200500000 | open | chr22 | 17000001 | 17250000 | closed |
| chr1 | 204500001 | 204750000 | open | chr22 | 22750001 | 2.30E+07 | open |
| chr1 | 212000001 | 212250000 | open | chr22 | 29000001 | 29250000 | open |
| chr1 | 217750001 | 2.18E+08 | closed | chr22 | 30500001 | 3.10E+07 | open |
| chr1 | 221000001 | 221250000 | closed | chr22 | 36250001 | 36500000 | open |
| chr1 | 228750001 | 2.29E+08 | open | chr22 | 36750001 | 3.70E+07 | open |
| chr1 | 229500001 | 229750000 | open | chr22 | 37500001 | 37750000 | open |
| chr1 | 231500001 | 231750000 | open | chr22 | 46500001 | 46750000 | closed |
| chr1 | 231750001 | 2.32E+08 | closed | chr3 | 250001 | 750000 | closed |
| chr1 | 234750001 | 2.35E+08 | open | chr3 | 1500001 | 1750000 | closed |
| chr1 | 235750001 | 2.36E+08 | open | chr3 | 2000001 | 2250000 | closed |
| chr1 | 238500001 | 239250000 | closed | chr3 | 14250001 | 14500000 | open |
| chr1 | 240250001 | 240500000 | closed | chr3 | 15250001 | 15500000 | open |
| chr1 | 243250001 | 243500000 | closed | chr3 | 20000001 | 20250000 | closed |
| chr1 | 244250001 | 244500000 | open | chr3 | 29000001 | 29250000 | closed |
| chr1 | 247250001 | 247500000 | closed | chr3 | 30500001 | 30750000 | closed |
| chr10 | 3500001 | 3750000 | closed | chr3 | 32250001 | 32500000 | open |
| chr10 | 3750001 | 4.00E+06 | open | chr3 | 37500001 | 37750000 | open |
| chr10 | 5750001 | 6.00E+06 | open | chr3 | 46000001 | 46250000 | open |
| chr10 | 6250001 | 6500000 | open | chr3 | 53000001 | 53250000 | open |
| chr10 | 8750001 | 9.00E+06 | closed | chr3 | 57000001 | 57250000 | open |
| chr10 | 11000001 | 11500000 | open | chr3 | 71500001 | 71750000 | closed |
| chr10 | 14500001 | 14750000 | open | chr3 | 71750001 | 7.20E+07 | open |
| chr10 | 17000001 | 17250000 | closed | chr3 | 72500001 | 72750000 | open |
| chr10 | 19750001 | 20250000 | closed | chr3 | 76750001 | 77500000 | closed |
| chr10 | 26250001 | 26750000 | open | chr3 | 79500001 | 79750000 | closed |
| chr10 | 27250001 | 27500000 | open | chr3 | 80000001 | 80250000 | closed |
| chr10 | 28250001 | 28500000 | closed | chr3 | 82750001 | 83500000 | closed |
| chr10 | 29500001 | 3.00E+07 | closed | chr3 | 83750001 | 85500000 | closed |
| chr10 | 30250001 | 30750000 | open | chr3 | 96750001 | 97250000 | closed |
| chr10 | 31500001 | 31750000 | open | chr3 | 101750001 | 1.02E+08 | open |
| chr10 | 43250001 | 43500000 | closed | chr3 | 103250001 | 103500000 | closed |
| chr10 | 48750001 | 4.90E+07 | open | chr3 | 112750001 | 1.13E+08 | open |
| chr10 | 50250001 | 50500000 | open | chr3 | 120000001 | 120250000 | open |
| chr10 | 55000001 | 55500000 | closed | chr3 | 122000001 | 122250000 | open |
| chr10 | 56500001 | 56750000 | closed | chr3 | 125000001 | 125250000 | open |
| chr10 | 57500001 | 57750000 | closed | chr3 | 126500001 | 126750000 | open |
| chr10 | 69000001 | 69250000 | open | chr3 | 130750001 | 1.31E+08 | open |
| chr10 | 73750001 | 7.40E+07 | open | chr3 | 133750001 | 1.34E+08 | open |
| chr10 | 75000001 | 75250000 | open | chr3 | 134500001 | 134750000 | open |
| chr10 | 75500001 | 75750000 | closed | chr3 | 141750001 | 1.42E+08 | open |
| chr10 | 77000001 | 77250000 | closed | chr3 | 149250001 | 149500000 | open |
| chr10 | 80000001 | 80250000 | open | chr3 | 152250001 | 152500000 | open |
| chr10 | 88000001 | 88250000 | open | chr3 | 155000001 | 155250000 | closed |
| chr10 | 88750001 | 89250000 | open | chr3 | 157250001 | 157500000 | closed |
| chr10 | 93250001 | 93500000 | open | chr3 | 162000001 | 162250000 | closed |
| chr10 | 95750001 | 9.60E+07 | open | chr3 | 163000001 | 165500000 | closed |
| chr10 | 97250001 | 97500000 | open | chr3 | 165750001 | 1.66E+08 | closed |
| chr10 | 99750001 | 1.00E+08 | open | chr3 | 166500001 | 1.67E+08 | closed |
| chr10 | 103000001 | 103250000 | open | chr3 | 169250001 | 169500000 | closed |
| chr10 | 115500001 | 115750000 | closed | chr3 | 171750001 | 172250000 | open |
| chr10 | 119250001 | 119500000 | open | chr3 | 177250001 | 177500000 | open |
| chr10 | 124000001 | 124250000 | open | chr3 | 184500001 | 184750000 | open |
| chr10 | 125000001 | 125250000 | closed | chr3 | 186000001 | 186500000 | open |
| chr10 | 127750001 | 1.28E+08 | closed | chr3 | 188000001 | 1.89E+08 | closed |
| chr10 | 132250001 | 132750000 | open | chr3 | 190750001 | 1.91E+08 | closed |
| chr11 | 6000001 | 6250000 | closed | chr3 | 192000001 | 192250000 | closed |
| chr11 | 13250001 | 13500000 | closed | chr3 | 194750001 | 195250000 | open |
| chr11 | 16000001 | 16750000 | closed | chr3 | 196500001 | 196750000 | open |
| chr11 | 24500001 | 24750000 | closed | chr4 | 1500001 | 2.00E+06 | open |
| chr11 | 25000001 | 25250000 | closed | chr4 | 10000001 | 10250000 | open |
| chr11 | 25500001 | 25750000 | closed | chr4 | 15500001 | 1.60E+07 | open |
| chr11 | 26250001 | 26500000 | closed | chr4 | 26000001 | 26250000 | open |
| chr11 | 35000001 | 35250000 | open | chr4 | 28000001 | 28250000 | closed |
| chr11 | 36250001 | 36500000 | closed | chr4 | 28500001 | 28750000 | closed |
| chr11 | 40750001 | 4.10E+07 | closed | chr4 | 29000001 | 30250000 | closed |
| chr11 | 47250001 | 47500000 | open | chr4 | 30750001 | 3.10E+07 | closed |
| chr11 | 48000001 | 48250000 | closed | chr4 | 31250001 | 31750000 | closed |
| chr11 | 49500001 | 49750000 | closed | chr4 | 32250001 | 32500000 | closed |
| chr11 | 57250001 | 57500000 | closed | chr4 | 32750001 | 3.30E+07 | closed |
| chr11 | 62250001 | 62500000 | open | chr4 | 34250001 | 35500000 | closed |
| chr11 | 66000001 | 66250000 | open | chr4 | 36000001 | 36250000 | open |
| chr11 | 68250001 | 68500000 | open | chr4 | 37750001 | 3.80E+07 | open |
| chr11 | 72500001 | 73500000 | open | chr4 | 38750001 | 3.90E+07 | open |
| chr11 | 75500001 | 75750000 | open | chr4 | 40500001 | 4.10E+07 | open |
| chr11 | 78250001 | 78500000 | open | chr4 | 48500001 | 48750000 | open |
| chr11 | 94500001 | 94750000 | closed | chr4 | 64250001 | 64500000 | closed |
| chr11 | 95000001 | 95250000 | open | chr4 | 65250001 | 66500000 | closed |
| chr11 | 95250001 | 95500000 | closed | chr4 | 70750001 | 7.10E+07 | open |
| chr11 | 96000001 | 96250000 | open | chr4 | 73750001 | 7.40E+07 | closed |
| chr11 | 97750001 | 98250000 | closed | chr4 | 78750001 | 7.90E+07 | open |
| chr11 | 98750001 | 9.90E+07 | closed | chr4 | 83000001 | 83250000 | open |
| chr11 | 99250001 | 100250000 | closed | chr4 | 89750001 | 9.00E+07 | closed |
| chr11 | 102500001 | 102750000 | closed | chr4 | 92500001 | 92750000 | closed |
| chr11 | 118000001 | 118250000 | open | chr4 | 93000001 | 93250000 | closed |
| chr11 | 120250001 | 120500000 | open | chr4 | 96750001 | 9.70E+07 | closed |
| chr11 | 121250001 | 121750000 | open | chr4 | 98500001 | 98750000 | open |
| chr11 | 126250001 | 126500000 | open | chr4 | 99750001 | 1.00E+08 | open |
| chr11 | 128500001 | 128750000 | open | chr4 | 102500001 | 102750000 | open |
| chr11 | 128750001 | 1.29E+08 | closed | chr4 | 104000001 | 104250000 | open |
| chr12 | 750001 | 1.00E+06 | open | chr4 | 105000001 | 105250000 | open |
| chr12 | 8750001 | 9.00E+06 | open | chr4 | 112250001 | 112500000 | open |
| chr12 | 10000001 | 10250000 | open | chr4 | 115750001 | 1.16E+08 | open |
| chr12 | 14500001 | 14750000 | open | chr4 | 116000001 | 116500000 | closed |
| chr12 | 17250001 | 17750000 | closed | chr4 | 117250001 | 117500000 | closed |
| chr12 | 26250001 | 26750000 | open | chr4 | 126250001 | 126500000 | closed |
| chr12 | 29000001 | 29250000 | closed | chr4 | 128250001 | 128500000 | open |
| chr12 | 31500001 | 31750000 | open | chr4 | 130750001 | 1.31E+08 | closed |
| chr12 | 32500001 | 32750000 | closed | chr4 | 132000001 | 1.33E+08 | closed |
| chr12 | 39500001 | 39750000 | closed | chr4 | 133250001 | 133500000 | closed |
| chr12 | 45000001 | 45250000 | closed | chr4 | 133750001 | 137500000 | closed |
| chr12 | 47500001 | 4.80E+07 | open | chr4 | 139750001 | 1.40E+08 | open |
| chr12 | 50500001 | 50750000 | open | chr4 | 143750001 | 144250000 | closed |
| chr12 | 51250001 | 51500000 | open | chr4 | 152500001 | 152750000 | open |
| chr12 | 54250001 | 54500000 | open | chr4 | 157250001 | 157500000 | closed |
| chr12 | 62250001 | 62500000 | closed | chr4 | 159000001 | 159250000 | open |
| chr12 | 64500001 | 64750000 | open | chr4 | 160250001 | 160750000 | closed |
| chr12 | 66000001 | 66250000 | open | chr4 | 161000001 | 161500000 | closed |
| chr12 | 69500001 | 69750000 | closed | chr4 | 161750001 | 162250000 | closed |
| chr12 | 73750001 | 74250000 | closed | chr4 | 164750001 | 1.65E+08 | open |
| chr12 | 76500001 | 76750000 | closed | chr4 | 165750001 | 1.66E+08 | closed |
| chr12 | 84000001 | 84250000 | closed | chr4 | 166750001 | 1.67E+08 | open |
| chr12 | 84500001 | 8.50E+07 | closed | chr4 | 169500001 | 169750000 | open |
| chr12 | 86000001 | 87250000 | closed | chr4 | 170750001 | 1.71E+08 | closed |
| chr12 | 89000001 | 89500000 | closed | chr4 | 174750001 | 1.75E+08 | closed |
| chr12 | 92000001 | 92250000 | open | chr4 | 175500001 | 1.76E+08 | closed |
| chr12 | 93250001 | 93500000 | open | chr4 | 176500001 | 176750000 | closed |
| chr12 | 94000001 | 94250000 | open | chr4 | 178000001 | 178500000 | closed |
| chr12 | 94500001 | 94750000 | open | chr4 | 179000001 | 1.80E+08 | closed |
| chr12 | 95250001 | 95500000 | open | chr4 | 180500001 | 1.81E+08 | closed |
| chr12 | 96000001 | 96250000 | open | chr4 | 181250001 | 181500000 | closed |
| chr12 | 102000001 | 102250000 | open | chr4 | 184250001 | 184500000 | open |
| chr12 | 109750001 | 1.10E+08 | open | chr4 | 184750001 | 1.85E+08 | open |
| chr12 | 110500001 | 110750000 | open | chr4 | 188750001 | 1.89E+08 | closed |
| chr12 | 112250001 | 112500000 | open | chr5 | 10500001 | 10750000 | open |
| chr12 | 116250001 | 116750000 | open | chr5 | 12250001 | 12500000 | closed |
| chr12 | 122750001 | 1.23E+08 | open | chr5 | 16500001 | 16750000 | closed |
| chr12 | 128750001 | 129250000 | closed | chr5 | 17250001 | 17500000 | open |
| chr12 | 129500001 | 129750000 | closed | chr5 | 19500001 | 2.00E+07 | closed |
| chr13 | 27000001 | 27250000 | open | chr5 | 22500001 | 22750000 | closed |
| chr13 | 28250001 | 28500000 | open | chr5 | 26000001 | 26250000 | closed |
| chr13 | 29750001 | 3.00E+07 | open | chr5 | 27500001 | 27750000 | closed |
| chr13 | 31750001 | 32250000 | open | chr5 | 39000001 | 39250000 | open |
| chr13 | 37000001 | 37250000 | closed | chr5 | 40250001 | 40750000 | open |
| chr13 | 40250001 | 40500000 | open | chr5 | 51250001 | 51500000 | closed |
| chr13 | 48000001 | 48250000 | open | chr5 | 51750001 | 5.20E+07 | closed |
| chr13 | 51500001 | 51750000 | open | chr5 | 52750001 | 5.30E+07 | closed |
| chr13 | 53750001 | 5.40E+07 | closed | chr5 | 54500001 | 5.50E+07 | closed |
| chr13 | 55750001 | 56250000 | closed | chr5 | 56000001 | 56750000 | open |
| chr13 | 62250001 | 62750000 | closed | chr5 | 59500001 | 59750000 | closed |
| chr13 | 68250001 | 68500000 | closed | chr5 | 61250001 | 61500000 | open |
| chr13 | 69000001 | 69500000 | closed | chr5 | 66750001 | 6.70E+07 | closed |
| chr13 | 69750001 | 70250000 | closed | chr5 | 67000001 | 67250000 | open |
| chr13 | 76750001 | 7.70E+07 | open | chr5 | 68250001 | 68500000 | open |
| chr13 | 77250001 | 77500000 | open | chr5 | 80250001 | 80500000 | open |
| chr13 | 82000001 | 82500000 | closed | chr5 | 85000001 | 85500000 | closed |
| chr13 | 83250001 | 83500000 | closed | chr5 | 85750001 | 86250000 | closed |
| chr13 | 84250001 | 84750000 | closed | chr5 | 87500001 | 87750000 | closed |
| chr13 | 95000001 | 95250000 | open | chr5 | 91750001 | 92250000 | closed |
| chr13 | 97250001 | 97500000 | open | chr5 | 99750001 | 1.00E+08 | closed |
| chr13 | 99250001 | 99500000 | open | chr5 | 101500001 | 101750000 | closed |
| chr13 | 103750001 | 104750000 | closed | chr5 | 104250001 | 104750000 | closed |
| chr13 | 105500001 | 105750000 | closed | chr5 | 105000001 | 106250000 | closed |
| chr13 | 107500001 | 1.08E+08 | closed | chr5 | 106500001 | 107250000 | closed |
| chr13 | 109500001 | 109750000 | closed | chr5 | 117250001 | 117500000 | closed |
| chr13 | 112750001 | 1.13E+08 | open | chr5 | 118000001 | 118500000 | closed |
| chr14 | 20000001 | 20250000 | closed | chr5 | 126750001 | 1.27E+08 | open |
| chr14 | 27000001 | 27500000 | closed | chr5 | 131750001 | 1.32E+08 | open |
| chr14 | 30500001 | 30750000 | closed | chr5 | 132500001 | 132750000 | open |
| chr14 | 30750001 | 31250000 | open | chr5 | 138750001 | 1.39E+08 | open |
| chr14 | 35250001 | 35500000 | open | chr5 | 142000001 | 142250000 | open |
| chr14 | 41750001 | 4.20E+07 | closed | chr5 | 142750001 | 143250000 | open |
| chr14 | 50750001 | 51250000 | open | chr5 | 148750001 | 1.49E+08 | closed |
| chr14 | 55250001 | 55500000 | open | chr5 | 150250001 | 150500000 | open |
| chr14 | 56000001 | 56500000 | closed | chr5 | 151000001 | 151250000 | open |
| chr14 | 59750001 | 6.00E+07 | closed | chr5 | 154750001 | 1.55E+08 | open |
| chr14 | 61500001 | 61750000 | open | chr5 | 156000001 | 156250000 | closed |
| chr14 | 63750001 | 64250000 | open | chr5 | 157250001 | 157500000 | open |
| chr14 | 64750001 | 6.50E+07 | open | chr5 | 158250001 | 158500000 | open |
| chr14 | 68500001 | 6.90E+07 | open | chr5 | 161250001 | 161500000 | open |
| chr14 | 70750001 | 71250000 | open | chr5 | 161750001 | 162250000 | closed |
| chr14 | 71500001 | 71750000 | open | chr5 | 162500001 | 1.63E+08 | closed |
| chr14 | 75250001 | 75500000 | open | chr5 | 164750001 | 1.65E+08 | closed |
| chr14 | 77750001 | 7.80E+07 | open | chr5 | 165500001 | 1.66E+08 | closed |
| chr14 | 80000001 | 80250000 | closed | chr5 | 166500001 | 1.67E+08 | closed |
| chr14 | 83250001 | 83500000 | closed | chr5 | 168750001 | 1.69E+08 | closed |
| chr14 | 89500001 | 89750000 | open | chr5 | 169500001 | 170500000 | open |
| chr14 | 90500001 | 90750000 | closed | chr5 | 177250001 | 177500000 | open |
| chr14 | 91000001 | 91250000 | open | chr6 | 1750001 | 2.00E+06 | open |
| chr14 | 92250001 | 92750000 | open | chr6 | 2750001 | 3.00E+06 | open |
| chr14 | 93000001 | 93250000 | open | chr6 | 3250001 | 3500000 | open |
| chr14 | 100250001 | 100500000 | open | chr6 | 4000001 | 4250000 | open |
| chr14 | 103250001 | 103500000 | open | chr6 | 4750001 | 5.00E+06 | open |
| chr14 | 104000001 | 104250000 | open | chr6 | 6750001 | 7.00E+06 | open |
| chr15 | 39250001 | 39500000 | closed | chr6 | 11250001 | 11750000 | open |
| chr15 | 40500001 | 40750000 | open | chr6 | 14750001 | 1.50E+07 | open |
| chr15 | 41750001 | 4.20E+07 | open | chr6 | 16500001 | 16750000 | open |
| chr15 | 42250001 | 42500000 | open | chr6 | 17750001 | 1.80E+07 | closed |
| chr15 | 43000001 | 43250000 | open | chr6 | 18250001 | 18500000 | closed |
| chr15 | 50000001 | 50250000 | closed | chr6 | 20250001 | 20500000 | open |
| chr15 | 51000001 | 51750000 | open | chr6 | 21500001 | 21750000 | open |
| chr15 | 52250001 | 52500000 | open | chr6 | 24750001 | 2.50E+07 | open |
| chr15 | 58000001 | 58250000 | closed | chr6 | 30500001 | 30750000 | open |
| chr15 | 59250001 | 59500000 | open | chr6 | 31250001 | 31750000 | open |
| chr15 | 62000001 | 62250000 | closed | chr6 | 32750001 | 3.30E+07 | open |
| chr15 | 63250001 | 63500000 | open | chr6 | 35000001 | 35500000 | open |
| chr15 | 64750001 | 6.50E+07 | open | chr6 | 37250001 | 37500000 | open |
| chr15 | 67000001 | 67250000 | open | chr6 | 42250001 | 42500000 | open |
| chr15 | 70500001 | 70750000 | open | chr6 | 45500001 | 45750000 | open |
| chr15 | 72000001 | 72500000 | open | chr6 | 49500001 | 49750000 | closed |
| chr15 | 77000001 | 77250000 | open | chr6 | 52250001 | 52500000 | open |
| chr15 | 79750001 | 8.00E+07 | open | chr6 | 57000001 | 57250000 | open |
| chr15 | 89750001 | 9.00E+07 | open | chr6 | 66500001 | 66750000 | closed |
| chr15 | 91750001 | 9.20E+07 | closed | chr6 | 67000001 | 67250000 | closed |
| chr15 | 94250001 | 94500000 | closed | chr6 | 74250001 | 74500000 | closed |
| chr15 | 100750001 | 1.01E+08 | closed | chr6 | 81750001 | 8.20E+07 | closed |
| chr15 | 101000001 | 101250000 | open | chr6 | 87750001 | 8.80E+07 | open |
| chr16 | 3000001 | 3250000 | open | chr6 | 89250001 | 89500000 | open |
| chr16 | 4250001 | 4500000 | open | chr6 | 92500001 | 92750000 | closed |
| chr16 | 10750001 | 1.10E+07 | open | chr6 | 93000001 | 93500000 | closed |
| chr16 | 11250001 | 11750000 | open | chr6 | 93750001 | 9.50E+07 | closed |
| chr16 | 12250001 | 12500000 | open | chr6 | 95250001 | 95500000 | closed |
| chr16 | 17000001 | 17500000 | open | chr6 | 96000001 | 96250000 | closed |
| chr16 | 23750001 | 24250000 | open | chr6 | 97750001 | 9.80E+07 | closed |
| chr16 | 27250001 | 27500000 | open | chr6 | 101750001 | 1.02E+08 | closed |
| chr16 | 29750001 | 3.00E+07 | open | chr6 | 102500001 | 103500000 | closed |
| chr16 | 31250001 | 31500000 | open | chr6 | 105750001 | 1.06E+08 | open |
| chr16 | 50500001 | 50750000 | open | chr6 | 106500001 | 106750000 | open |
| chr16 | 53750001 | 5.40E+07 | closed | chr6 | 109750001 | 1.10E+08 | open |
| chr16 | 62250001 | 62500000 | closed | chr6 | 114500001 | 114750000 | closed |
| chr16 | 63250001 | 63750000 | closed | chr6 | 115750001 | 1.16E+08 | closed |
| chr16 | 66500001 | 66750000 | open | chr6 | 134250001 | 134500000 | open |
| chr16 | 79000001 | 79250000 | closed | chr6 | 137000001 | 137500000 | open |
| chr16 | 81250001 | 8.20E+07 | open | chr6 | 137750001 | 1.38E+08 | open |
| chr16 | 83750001 | 8.40E+07 | closed | chr6 | 143500001 | 143750000 | closed |
| chr16 | 84250001 | 84500000 | closed | chr6 | 144000001 | 144750000 | open |
| chr16 | 84500001 | 85250000 | open | chr6 | 152250001 | 152500000 | closed |
| chr17 | 2750001 | 3.00E+06 | closed | chr6 | 153250001 | 1.54E+08 | closed |
| chr17 | 3750001 | 4.00E+06 | open | chr6 | 154250001 | 154500000 | closed |
| chr17 | 5500001 | 5750000 | closed | chr6 | 159750001 | 1.60E+08 | open |
| chr17 | 8750001 | 9.00E+06 | open | chr6 | 164750001 | 1.65E+08 | closed |
| chr17 | 9250001 | 9500000 | closed | chr6 | 166250001 | 166750000 | open |
| chr17 | 10000001 | 10250000 | closed | chr6 | 168750001 | 1.69E+08 | closed |
| chr17 | 20000001 | 20250000 | closed | chr7 | 7000001 | 7250000 | closed |
| chr17 | 29500001 | 29750000 | open | chr7 | 8750001 | 9250000 | closed |
| chr17 | 31000001 | 31250000 | open | chr7 | 9500001 | 1.00E+07 | closed |
| chr17 | 44250001 | 44500000 | open | chr7 | 10500001 | 10750000 | closed |
| chr17 | 45250001 | 45500000 | open | chr7 | 14000001 | 14500000 | closed |
| chr17 | 47000001 | 47250000 | open | chr7 | 27750001 | 28250000 | closed |
| chr17 | 56750001 | 5.70E+07 | open | chr7 | 30750001 | 3.10E+07 | closed |
| chr17 | 57750001 | 5.80E+07 | open | chr7 | 36000001 | 36250000 | closed |
| chr17 | 61750001 | 6.20E+07 | open | chr7 | 36500001 | 3.70E+07 | closed |
| chr17 | 64250001 | 64750000 | open | chr7 | 37500001 | 37750000 | closed |
| chr17 | 66250001 | 66500000 | closed | chr7 | 48250001 | 48500000 | closed |
| chr17 | 67250001 | 67750000 | open | chr7 | 53500001 | 53750000 | open |
| chr17 | 68250001 | 68500000 | open | chr7 | 77250001 | 77750000 | closed |
| chr17 | 72750001 | 7.30E+07 | closed | chr7 | 83000001 | 83250000 | closed |
| chr17 | 74500001 | 74750000 | open | chr7 | 83500001 | 83750000 | closed |
| chr17 | 75250001 | 75500000 | open | chr7 | 86000001 | 86250000 | closed |
| chr17 | 77250001 | 77750000 | open | chr7 | 89000001 | 89500000 | closed |
| chr17 | 78750001 | 7.90E+07 | open | chr7 | 89750001 | 9.00E+07 | closed |
| chr17 | 81000001 | 81250000 | open | chr7 | 100250001 | 100500000 | open |
| chr18 | 250001 | 5.00E+05 | closed | chr7 | 106250001 | 106500000 | open |
| chr18 | 9500001 | 9750000 | open | chr7 | 107000001 | 107250000 | closed |
| chr18 | 24000001 | 24250000 | open | chr7 | 110000001 | 110250000 | closed |
| chr18 | 24250001 | 24500000 | closed | chr7 | 117000001 | 117250000 | closed |
| chr18 | 30000001 | 30250000 | closed | chr7 | 118750001 | 120500000 | closed |
| chr18 | 44750001 | 45250000 | open | chr7 | 124500001 | 124750000 | closed |
| chr18 | 57500001 | 57750000 | open | chr7 | 125750001 | 1.26E+08 | closed |
| chr18 | 63000001 | 63250000 | open | chr7 | 126250001 | 126500000 | closed |
| chr18 | 63750001 | 6.40E+07 | open | chr7 | 129750001 | 1.30E+08 | open |
| chr18 | 64750001 | 65750000 | closed | chr7 | 130750001 | 1.31E+08 | closed |
| chr18 | 66000001 | 66250000 | closed | chr7 | 131000001 | 131250000 | open |
| chr18 | 66500001 | 6.70E+07 | closed | chr7 | 136250001 | 136500000 | closed |
| chr18 | 67750001 | 68250000 | closed | chr7 | 139750001 | 1.40E+08 | closed |
| chr18 | 72000001 | 72250000 | closed | chr7 | 142000001 | 142250000 | closed |
| chr18 | 76250001 | 76500000 | open | chr7 | 143250001 | 143500000 | closed |
| chr18 | 76500001 | 76750000 | closed | chr7 | 145250001 | 145750000 | closed |
| chr19 | 2500001 | 2750000 | open | chr7 | 147000001 | 147500000 | closed |
| chr19 | 6750001 | 7.00E+06 | open | chr8 | 3500001 | 4.00E+06 | closed |
| chr19 | 7500001 | 7750000 | open | chr8 | 4250001 | 4500000 | closed |
| chr19 | 14250001 | 14750000 | open | chr8 | 4750001 | 5.00E+06 | closed |
| chr19 | 16000001 | 16500000 | open | chr8 | 8750001 | 9.00E+06 | open |
| chr19 | 16750001 | 1.70E+07 | open | chr8 | 10000001 | 10250000 | closed |
| chr19 | 21500001 | 21750000 | closed | chr8 | 15000001 | 15500000 | closed |
| chr19 | 32250001 | 32500000 | closed | chr8 | 19250001 | 19750000 | closed |
| chr19 | 33250001 | 33500000 | closed | chr8 | 20000001 | 20250000 | closed |
| chr19 | 35000001 | 35250000 | closed | chr8 | 22000001 | 22250000 | open |
| chr19 | 35250001 | 35500000 | open | chr8 | 23000001 | 23250000 | open |
| chr19 | 41250001 | 4.20E+07 | open | chr8 | 29000001 | 29250000 | open |
| chr19 | 42500001 | 42750000 | closed | chr8 | 29500001 | 29750000 | open |
| chr19 | 43250001 | 43500000 | closed | chr8 | 38750001 | 3.90E+07 | open |
| chr19 | 43500001 | 43750000 | open | chr8 | 42750001 | 4.30E+07 | open |
| chr19 | 45500001 | 45750000 | open | chr8 | 47250001 | 47750000 | open |
| chr19 | 53750001 | 54500000 | closed | chr8 | 50750001 | 5.10E+07 | closed |
| chr19 | 54750001 | 5.50E+07 | closed | chr8 | 55750001 | 5.60E+07 | open |
| chr2 | 8000001 | 8500000 | open | chr8 | 58000001 | 58250000 | open |
| chr2 | 16500001 | 16750000 | closed | chr8 | 60750001 | 6.10E+07 | open |
| chr2 | 23750001 | 2.40E+07 | open | chr8 | 67250001 | 67500000 | open |
| chr2 | 25250001 | 25500000 | open | chr8 | 73500001 | 73750000 | open |
| chr2 | 28250001 | 28500000 | open | chr8 | 75750001 | 76500000 | closed |
| chr2 | 30250001 | 30500000 | open | chr8 | 77250001 | 77500000 | closed |
| chr2 | 31000001 | 31250000 | open | chr8 | 81000001 | 81250000 | open |
| chr2 | 33000001 | 33250000 | closed | chr8 | 83000001 | 83250000 | closed |
| chr2 | 34750001 | 35250000 | closed | chr8 | 83500001 | 83750000 | closed |
| chr2 | 35500001 | 35750000 | closed | chr8 | 85250001 | 85500000 | closed |
| chr2 | 37250001 | 38250000 | open | chr8 | 99000001 | 99250000 | open |
| chr2 | 38500001 | 38750000 | open | chr8 | 100250001 | 100500000 | open |
| chr2 | 42000001 | 42250000 | open | chr8 | 110250001 | 110500000 | closed |
| chr2 | 43000001 | 43250000 | open | chr8 | 110750001 | 1.12E+08 | closed |
| chr2 | 46500001 | 46750000 | open | chr8 | 112250001 | 114750000 | closed |
| chr2 | 48000001 | 48250000 | open | chr8 | 123000001 | 123250000 | open |
| chr2 | 51250001 | 51750000 | closed | chr8 | 125500001 | 125750000 | open |
| chr2 | 53750001 | 5.40E+07 | open | chr8 | 127250001 | 127500000 | open |
| chr2 | 54500001 | 54750000 | open | chr8 | 129750001 | 1.30E+08 | open |
| chr2 | 56750001 | 5.70E+07 | closed | chr8 | 132500001 | 132750000 | closed |
| chr2 | 64000001 | 64250000 | open | chr8 | 132750001 | 133250000 | open |
| chr2 | 64750001 | 6.50E+07 | open | chr8 | 136250001 | 136750000 | closed |
| chr2 | 69000001 | 69250000 | open | chr8 | 140000001 | 140250000 | open |
| chr2 | 69750001 | 7.00E+07 | open | chr8 | 141000001 | 141250000 | open |
| chr2 | 71500001 | 71750000 | closed | chr9 | 11750001 | 12250000 | closed |
| chr2 | 74750001 | 7.50E+07 | open | chr9 | 21000001 | 21250000 | open |
| chr2 | 77000001 | 77500000 | closed | chr9 | 25000001 | 25250000 | closed |
| chr2 | 78250001 | 78750000 | closed | chr9 | 27500001 | 27750000 | closed |
| chr2 | 85500001 | 8.60E+07 | open | chr9 | 68750001 | 6.90E+07 | open |
| chr2 | 98500001 | 9.90E+07 | open | chr9 | 71500001 | 71750000 | closed |
| chr2 | 100000001 | 100250000 | open | chr9 | 75000001 | 75250000 | closed |
| chr2 | 101000001 | 101500000 | open | chr9 | 76500001 | 76750000 | closed |
| chr2 | 102250001 | 102500000 | open | chr9 | 87750001 | 8.80E+07 | closed |
| chr2 | 105750001 | 1.06E+08 | open | chr9 | 89500001 | 9.00E+07 | open |
| chr2 | 109250001 | 109500000 | open | chr9 | 90750001 | 9.10E+07 | open |
| chr2 | 113000001 | 113250000 | open | chr9 | 93000001 | 93250000 | open |
| chr2 | 113750001 | 1.14E+08 | open | chr9 | 97500001 | 97750000 | open |
| chr2 | 115750001 | 1.16E+08 | closed | chr9 | 99000001 | 99250000 | open |
| chr2 | 116500001 | 116750000 | closed | chr9 | 102500001 | 102750000 | closed |
| chr2 | 126500001 | 126750000 | closed | chr9 | 103000001 | 103250000 | closed |
| chr2 | 128500001 | 128750000 | closed | chr9 | 103500001 | 103750000 | closed |
| chr2 | 134000001 | 134500000 | open | chr9 | 104750001 | 105250000 | closed |
| chr2 | 136000001 | 136500000 | open | chr9 | 107500001 | 1.08E+08 | closed |
| chr2 | 139000001 | 139250000 | closed | chr9 | 109000001 | 109250000 | open |
| chr2 | 139750001 | 1.40E+08 | closed | chr9 | 110000001 | 110250000 | open |
| chr2 | 140250001 | 140750000 | closed | chr9 | 112750001 | 1.13E+08 | closed |
| chr2 | 143000001 | 143250000 | open | chr9 | 114250001 | 114500000 | open |
| chr2 | 144500001 | 144750000 | closed | chr9 | 124000001 | 124500000 | open |
| chr2 | 153750001 | 1.54E+08 | closed | chr9 | 126250001 | 126500000 | open |
| chr2 | 155500001 | 155750000 | closed | chr9 | 129250001 | 129500000 | open |
| chr2 | 157250001 | 157500000 | open | chr9 | 131500001 | 131750000 | open |
| | | | | chr9 | 136500001 | 136750000 | open |

2. Enrichment of Desired Regions

Enrichment of the sample for selected areas of interest may optionally be performed (e.g., using a suitable technique such as hybrid selection with bait DNA sequences). This technique may be performed before or after preparation for measurement in Step 3. For example, if an Illumina DNA sequencer is used to quantify fragment lengths, sequencing library preparation could be performed prior to enrichment. If capillary electrophoresis length analysis is used, no such preparation may be needed before enrichment.

Enrichment of different regions may be performed in several steps, resulting in several enriched DNA pools from the input pool. For example, an enrichment may be performed for promoter regions of the genome separately from an enrichment of bulk genomic windows.

3. Measurement of Fragment Ratio in Desired Regions

For each enrichment pool and/or unenriched pool, fragment lengths are profiled. This is a measurement of the count of fragments in at least two bins, or a measurement of the relative abundance of the two bins. For example, a measurement technique may get the ratio of abundance of fragments with length 100 bp to 149 bp over the abundance of fragments with length 150 bp to 220 bp. This may be an exact measurement of every fragment's length at base level precision.

In addition to measuring the overall lengths of a pool, processing of the measurements may filter the DNA fragments, for example to limit analysis to certain areas of the genome, or to fragments that meet certain quality control requirements. When using DNA sequencing technology to measure fragment lengths, DNA fragments may be mapped to the genome, and analysis may be restricted to an opening region or a closed region of the genome, as determined from the selection in Step 1.

DNA fragment length measurement technologies may include DNA sequencing, capillary electrophoresis, SPRI bead selection and quantification, or other techniques.

When performing DNA sequencing, the sequencing depth can be determined based on prior sequencing results, and how much the target region has been restricted via step 1. Using no enrichment at all with the RS detector described in Example 3, clear separation may be established with only 5 million reads of sequencing. However, with more focused sequencing, fewer reads may be required.

4. Assessment of Sample Composition from Fragment Measurements

From each sample, one or more DNA pools potentially enriched for a subset of a reference genome are received. From each DNA pool, at least one “fragment length profile” which could range from a single number to a vector of numbers is received. Further, the fragment length profile from each DNA pool may be broken down into genome-specific regions, such as 250 kb windows along the genome.

This set of measurements for a sample is compared to a database of expected measurements of potential cell types that are reported back to the user. This comparison can take the form of any number of computational steps, such as T-tests between distributions of observed fragment length ratios and/or regression of a vector of the observed measurements as a linear combination of expected measurements from the cells in the cell type database. T-tests operate on windows that have been pre-selected as informative for the comparison of interest, or may include all regions of the genome. Each T-Test is then performed between the sample of interest and the known database samples to establish similarity or dissimilarity to the samples in the database. For regression, the vector of genomic window fragment ratios is represented as a weighted sum of samples in the database. Regression in this case may be restricted to positive contributions from the cell types, with total contributions equaling 1. This regression also may be regularized with penalizers such as an L1 or L2, to make the selection of samples more sparse.

In the example RS detector discussed in Example 3, T-tests between different genomic regions, from a single unenriched DNA pool, were used to separate out the RS-differential samples on a 2D plane using the two different T-statistics from each test. A decision line is drawn to more clearly separate the two classes

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%).

Certain implementations of the technology are set forth in the claim(s) that follow(s).

What is claimed is:

1. A method for detecting the presence or absence of a population of cells in a subject comprising:
a) determining lengths of nucleic acid fragments in a test sample from a subject; and
b) detecting the presence or absence of a population of cells in a subject according the nucleic acid fragment lengths determined in (a).

2. The method of claim 1, further comprising obtaining nucleic acid sequence reads of the nucleic acid fragments.

3. The method of claim 1, wherein the nucleic acid fragment lengths are determined from the nucleic acid sequence reads.

4. The method of any one of claims 1-3, further comprising prior to (a) sequencing nucleic acids in the test sample by a sequencing process, thereby generating the nucleic acid sequence reads.

5. The method of claim 4, wherein the sequencing process comprises single-end sequencing, and the nucleic acid fragment lengths are determined according to the length of a single-end sequencing read.

6. The method of claim 4, wherein the sequencing process comprises paired-end sequencing, and the nucleic acid fragment lengths are determined according to mapped positions of paired-end sequencing reads.

7. The method of claim 1, further comprising prior to (a), performing capillary electrophoresis on the nucleic acid fragments in the test sample, wherein the nucleic acid fragment lengths are determined according to the capillary electrophoresis.

8. The method of any one of claims 1-7, further comprising after (a) generating fragment counts.

9. The method of claim 8, wherein detecting the presence or absence of a population of cells in a subject in (b) is according to the fragment counts.

10. The method of any one of claims 1-9, further comprising after (a) generating one or more fragment length ratios.

11. The method of claim 10, wherein each of the one or more fragment length ratios is a ratio chosen from short fragment counts to long fragment counts, short fragment counts to medium fragment counts, and medium fragment counts to long fragment counts.

12. The method of claim 11, wherein the short fragments comprise fragments that are about 0 bp to about 99 bp in length, the medium fragments comprise fragments that are about 100 bp to about 149 bp in length, and the long fragments comprise fragments that are about 150 bp to about 220 bp in length.

13. The method of any one of claims 10-12, wherein the one or more fragment length ratios are generated for one or more genomic portions.

14. The method of claim 13, wherein the one or more genomic portions are about 50 kb to about 500 kb in length.

15. The method of claim 13, wherein the one or more genomic portions are about 250 kb in length.

16. The method of any one of claims 10-15, wherein detecting the presence or absence of a population of cells in a subject in (b) is according to the one or more fragment length ratios.

17. The method of any one of claims 1-16, further comprising comparing the nucleic acid fragment lengths, or a derivative thereof, for the test sample to nucleic acid fragment lengths, or a derivative thereof, for one or more control samples, thereby generating a comparison.

18. The method of claim 17, wherein detecting the presence or absence of a population of cells in a subject in (b) is according to the comparison.

19. The method of any one of claims 1-18, further comprising enriching the test sample for one or more selected genomic regions, thereby generating one or more pools of enriched nucleic acid.

20. The method of claim 19, wherein the one or more genomic regions are selected according to A/B status for a chosen cell type.

21. The method of claim 20, wherein the one or more genomic regions are selected according to A/B status differences between a diseased cell and a non-diseased cell.

22. The method of any one of claims 19-21, wherein the enriching is performed prior to (a).

23. The method of any one of claims 19-22, wherein the enriching comprises hybridizing capture probes to nucleic acid fragments in the test sample, wherein the capture probes are specific to the one or more selected genomic regions.

24. The method of any one of claims 1-23, further comprising identifying an A/B status of the genome, or portion thereof, for the test sample according to the nucleic acid fragment lengths determined in (a).

25. The method of claim 24, further comprising comparing the A/B status of the genome, or portion thereof, for the test sample to an A/B status of a control genome, thereby generating an A/B status comparison.

26. The method of claim 25, wherein detecting the presence or absence of a population of cells in a subject in (b) is according to the A/B status comparison.

27. The method of any one of claims 1-26, wherein the test sample comprises cell-free DNA (cfDNA).

28. The method of any one of claims 1-26, wherein the test sample comprises circulating tumor DNA (ctDNA).

29. The method of any one of claims 1-28, wherein the population of cells in (b) comprises diseased cells.

30. The method of any one of claims 1-29, wherein the population of cells in (b) comprises cancer cells.

31. The method of any one of claims 1-30, further comprising detecting a presence or absence of a disease or a disease subtype for the subject according to the presence or absence of the population of cells detected in (b).

32. The method of claim 31, wherein the disease is cancer.

33. The method of claim 32, wherein the disease is MDS.

34. The method claim 32 or 33, wherein the disease is MDS-RS-MLD.


SEQUENCE LISTING (2 sequences)

SEQ ID NO: 1 - DNA, 10 bp, Artificial Sequence
  Description of Artificial Sequence Synthetic oligonucleotide
ggcccgacgg

SEQ ID NO: 2 - DNA, 20 bp, Artificial Sequence
  Description of Artificial Sequence Synthetic oligonucleotide
tttttttttttttttttttt